SEQUENCE LISTING (152 sequences)

SEQ ID NO: 1 - protein, 47 aa, Artificial Sequence
  Description of Artificial Sequence Synthetic polypeptide
LEIRAAFLRQRNTALRTEVAELEQEVQRLENEVSQYETRYGPLGGGK

SEQ ID NO: 2 - protein, 47 aa, Artificial Sequence
  Description of Artificial Sequence Synthetic polypeptide
LEIEAAFLERENTALETRVAELRQRVQRLRNRVSQYRTRYGPLGGGK

SEQ ID NO: 3 - protein, 19 aa, Artificial Sequence
  Description of Artificial Sequence Synthetic peptide
GGGGSGGGGSGGGGSGGGS

SEQ ID NO: 4 - protein, 38 aa, Artificial Sequence
  Description of Artificial Sequence Synthetic polypeptide
GSQKVESLKQKIEELKQRKAQLKNDIANLEKEIAYAET

SEQ ID NO: 5 - protein, 44 aa, Artificial Sequence
  Description of Artificial Sequence Synthetic polypeptide
GSELTDTLQAETDQLEDEKSALQTEIANLLKEKEKLEFILAAHR

SEQ ID NO: 6 - protein, 44 aa, Artificial Sequence
  Description of Artificial Sequence Synthetic polypeptide
ERISRLEEKVKTLKSQNTELASTASLLREQVAQLKQKVLSHVLE

SEQ ID NO: 7 - protein, 9 aa, Artificial Sequence
  Description of Artificial Sequence Synthetic peptide
CHTQSSPKL

SEQ ID NO: 8 - protein, 13 aa, Artificial Sequence
  Description of Artificial Sequence Synthetic peptide
VPRDCGCKPCICT

SEQ ID NO: 9 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 10 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 11 - protein, 25 aa, Unknown
  Description of Unknown CD8 sequence
IYIWAPLAGICVALLLSLIITLICY

SEQ ID NO: 12 - protein, 21 aa, Unknown
  Description of Unknown CD8 sequence
IYIWAPLAGTCGVLLLSLVIT

SEQ ID NO: 13 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 14 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 15 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 17 - protein, 113 aa, Unknown
  Description of Unknown CD3-zeta sequence
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 18 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 19 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 20 - protein, 15 aa, Artificial Sequence
  Description of Artificial Sequence Synthetic peptide
GGGGSGGGGSGGGGS

SEQ ID NO: 21 - protein, 12 aa, Artificial Sequence
  Description of Artificial Sequence Synthetic peptide
CPSPLFPGPSKP

SEQ ID NO: 22 - protein, 14 aa, Artificial Sequence
  Description of Artificial Sequence Synthetic peptide
EPKSCDKTHTCPPC

SEQ ID NO: 23 - protein, 27 aa, Unknown
  Description of Unknown CD28 sequence
FWALVVVAGVLFCYGLLVTVALCVIWT

SEQ ID NO: 24 - protein, 325 aa, Artificial Sequence
  Description of Artificial Sequence Synthetic polypeptide
METDTLLLWVLLLWVPGSTGGSGGKPIPNPLLGLDSTGGGGSGGGSLEIEAAFLERENTALETRVAELRQRVQRLRNRVSQYRTRYGPLGGGKAAACHTQSSPKLGSLFYVTVGVGAGGLLLVLLVALFIFCICKRRKGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 25 - protein, 355 aa, Artificial Sequence
  Description of Artificial Sequence Synthetic polypeptide
METDTLLLWVLLLWVPGSTGGSGGKPIPNPLLGLDSTGGGGSGGGSLEIEAAFLERENTALETRVAELRQRVQRLRNRVSQYRTRYGPLGGGKAAACHTQSSPKLGSLFWALVVVAGVLFCYGLLVTVALCVIWTRAKFSRSAETAANLQDTFDALHMQTLAPRRAKRGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 26 - protein, 312 aa, Artificial Sequence
  Description of Artificial Sequence Synthetic polypeptide
METDTLLLWVLLLWVPGSTGLEIEAAFLERENTALETRVAELRQRVQRLRNRVSQYRTRYGPLGGGKGSGEQKLISEEDLAAACHTQSSPKLGSLFYVTVGVGAGGLLLVLLVALFIFCICKRRKGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 27 - protein, 342 aa, Artificial Sequence
  Description of Artificial Sequence Synthetic polypeptide
METDTLLLWVLLLWVPGSTGLEIEAAFLERENTALETRVAELRQRVQRLRNRVSQYRTRYGPLGGGKGSGEQKLISEEDLAAACHTQSSPKLGSLFWALVVVAGVLFCYGLLVTVALCVIWTRAKFSRSAETAANLQDTFDALHMQTLAPRRAKRGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 28 - protein, 329 aa, Artificial Sequence
  Description of Artificial Sequence Synthetic polypeptide
METDTLLLWVLLLWVPGSTGGSGGKPIPNPLLGLDSTGGGGSGGGSLEIEAAFLERENTALETRVAELRQRVQRLRNRVSQYRTRYGPLGGGKAAAVPRDCGCKPCICTGSLFYVTVGVGAGGLLLVLLVALFIFCICKRRKGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 29 - protein, 359 aa, Artificial Sequence
  Description of Artificial Sequence Synthetic polypeptide
METDTLLLWVLLLWVPGSTGGSGGKPIPNPLLGLDSTGGGGSGGGSLEIEAAFLERENTALETRVAELRQRVQRLRNRVSQYRTRYGPLGGGKAAAVPRDCGCKPCICTGSLFWALVVVAGVLFCYGLLVTVALCVIWTRAKFSRSAETAANLQDTFDALHMQTLAPRRAKRGTGQCTNYALLKLAGDVESNPGPGSMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 30 - protein, 735 aa, Artificial Sequence
  Description of Artificial Sequence Synthetic polypeptide
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRDGSGRAKRATNFSLLKQAGDVEENPGPGSLMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 31 - protein, 537 aa, Artificial Sequence
  Description of Artificial Sequence Synthetic polypeptide
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 32 - protein, 362 aa, Artificial Sequence
  Description of Artificial Sequence Synthetic polypeptide
METDTLLLWVLLLWVPGSTGLEIEAAFLERENTALETRVAELRQRVQRLRNRVSQYRTRYGPLGGGKEFTEQKLISEEDLVIIAVLVVLGAAIVTGAVVAFVMKGSGATNFSLLKQAGDVEENPGPGSLMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 33 - protein, 540 aa, Artificial Sequence
  Description of Artificial Sequence Synthetic polypeptide
METDTLLLWVLLLWVPGSTGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSL

SEQ ID NO: 34 - protein, 541 aa, Artificial Sequence
  Description of Artificial Sequence Synthetic polypeptide
METDTLLLWVLLLWVPGSTGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLT

SEQ ID NO: 35 - protein, 242 aa, Artificial Sequence
  Description of Artificial Sequence Synthetic polypeptide
EQKLISEEDLLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 36 - protein, 940 aa, Artificial Sequence
  Description of Artificial Sequence Synthetic polypeptide
METDTLLLWVLLLWVPGSTGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLTMASPLTRFLSLNLLLLGESIILGSGEAASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVECLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 37 - protein, 541 aa, Artificial Sequence; identical to SEQ ID NO: 34
  Description of Artificial Sequence Synthetic polypeptide

SEQ ID NO: 38 - protein, 393 aa, Artificial Sequence
  Description of Artificial Sequence Synthetic polypeptide
GTGGSTGGGGSGGGGSGGGGSASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVECLESSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGTEARL

SEQ ID NO: 39 - protein, 305 aa, Artificial Sequence
  Description of Artificial Sequence Synthetic polypeptide
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLT

SEQ ID NO: 40 - protein, 291 aa, Artificial Sequence
  Description of Artificial Sequence Synthetic polypeptide
SAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 41 - protein, 305 aa, Artificial Sequence; identical to SEQ ID NO: 39
  Description of Artificial Sequence Synthetic polypeptide

SEQ ID NO: 42 - protein, 484 aa, Artificial Sequence
  Description of Artificial Sequence Synthetic polypeptide
SAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 43 - protein, 305 aa, Artificial Sequence; identical to SEQ ID NO: 39
  Description of Artificial Sequence Synthetic polypeptide

SEQ ID NO: 44 - protein, 469 aa, Artificial Sequence
  Description of Artificial Sequence Synthetic polypeptide
SAWSHPQFEKGGGSGGGSGGSAWSHPQFEKGGGGSGGGSEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLESMYSMQLASCVTLTLVLLVNSNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 45 - protein, 305 aa, Artificial Sequence; identical to SEQ ID NO: 39
  Description of Artificial Sequence Synthetic polypeptide

SEQ ID NO: 46 - protein, 21 aa, Artificial Sequence
  Description of Artificial Sequence Synthetic peptide
GTGGSTGGGGSGGGGSGGGGS

SEQ ID NO: 47 - protein, 262 aa, Artificial Sequence
  Description of Artificial Sequence Synthetic polypeptide
EQKLISEEDLEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 48 - protein, 305 aa, Artificial Sequence; identical to SEQ ID NO: 39
  Description of Artificial Sequence Synthetic polypeptide

SEQ ID NO: 49 - protein, 452 aa, Artificial Sequence
  Description of Artificial Sequence Synthetic polypeptide
GTGGSTGGGGSGGGGSGGGGSASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVECSAWSHPQFEKGGGSGGGSGGSAWSHPQFEKGGGGSGGGGEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 50 - protein, 496 aa, Artificial Sequence
  Description of Artificial Sequence Synthetic polypeptide
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISLGSGATNFSLLKQAGDVEENPGPGSLTMASPLTRFLSLNLLLLGESIILGSGEAASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVECGGGGSGGGGSGGGGSGGGGSGGGS

SEQ ID NO: 51 - protein, 291 aa, Artificial Sequence
  Description of Artificial Sequence Synthetic polypeptide
SAWSHPQFEKGGGSGGGSGGSAWSHPQFEKGGGGSGGGGEARLSTTTKPVLRTPSPVHPTGTSQPQRPEDCRPRGSVKGTGLDFACDIYIWAPLAGICVALLLSLIITLICYNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRGSGQCTNYALLKLAGDVESNPGPLE

SEQ ID NO: 52 - protein, 687 aa, Artificial Sequence
  Description of Artificial Sequence Synthetic polypeptide
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSIGGGGSGGGSALGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLT

SEQ ID NO: 53 - protein, 242 aa, Artificial Sequence; identical to SEQ ID NO: 35
  Description of Artificial Sequence Synthetic polypeptide

SEQ ID NO: 54 - protein, 667 aa, Artificial Sequence
  Description of Artificial Sequence Synthetic polypeptide
MYSMQLASCVTLTLVLLVNSNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTSGGGGSGGGSALGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLT

SEQ ID NO: 55 - protein, 242 aa, Artificial Sequence; identical to SEQ ID NO: 35
  Description of Artificial Sequence Synthetic polypeptide

SEQ ID NO: 56 - protein, 682 aa, Artificial Sequence
  Description of Artificial Sequence Synthetic polypeptide
MYSMQLASCVTLTLVLLVNSHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLSGGGGSGGGSALGDYKDDDDKLEIRAAFLRQRNTALRTEVAELEQEVQRLENEVSQYETRYGPLGGGKGSGRAKRATNFSLLKQAGDVEENPGPEFTRAKRMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVDGTGRAKRVKQTLNFDLLKLAGDVESNPGPGSLT

SEQ ID NO: 57 - protein, 242 aa, Artificial Sequence; identical to SEQ ID NO: 35
  Description of Artificial Sequence Synthetic polypeptide

SEQ ID NO: 58 - protein, 21 aa, Artificial Sequence
  Description of Artificial Sequence Synthetic peptide
GTGGSTGGGGSGGGGSGGGGS

SEQ ID NO: 59 - protein, 24 aa, Artificial Sequence
  Description of Artificial Sequence Synthetic peptide
GGGGSGGGGSGGGGSGGGGSGGGS

SEQ ID NO: 60 - protein, 140 aa, Mus musculus
ASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVEC

SEQ ID NO: 61 - protein, 133 aa, Homo sapiens
APMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 62 - protein, 44 aa, Mus musculus
STTTKPVLRTPSPVHPTGTSQPQRPEDCRPRGSVKGTGLDFACD

SEQ ID NO: 63 - protein, 45 aa, Homo sapiens
TTTPAPRPPTPAPTIASQPLSLRPEACRPAAGGAVHTRGLDFACD

SEQ ID NO: 64 - protein, 36 aa, Mus musculus
IEFMYPPPYLDNERSNGTIIHIKEKHLCHTQSSPKL

SEQ ID NO: 65 - protein, 39 aa, Homo sapiens
IEVMYPPPYLDNEKSNGTIIHVKGKHLCPSPLFPGPSKP

SEQ ID NO: 66 - protein, 112 aa, Mus musculus
QKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKC

SEQ ID NO: 67 - protein, 31 aa, Mus musculus
GGISLLVQNTSWMLLLLLSLSLLQALDFISL

SEQ ID NO: 68 - protein, 111 aa, Homo sapiens
QKVTSLTACLVDQSLRLDCRHENTSSSPIQYEFSLTRETKKHVLFGTVGVPEHTYRSRTNFTSKYNMKVLYLSAFTSKDEGTYTCALHHSGHSPPISSQNVTVLRDKLVKC

SEQ ID NO: 69 - protein, 31 aa, Homo sapiens
EGISLLAQNTSWLLLLLLSLSLLQATDFMSL

SEQ ID NO: 70 - protein, 312 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 71 - protein, 23 aa, Homo sapiens
IATGMVGALLLLLVVALGIGLFM

SEQ ID NO: 72 - protein, 30 aa, Mus musculus
FYVTVGVGAGGLLLVLLVALFIFCICKRRK

SEQ ID NO: 73 - protein, 30 aa, Homo sapiens
IYLIIGICGGGSLLMVFVALLVFYITKRKK

SEQ ID NO: 74 - protein, 27 aa, Mus musculus
VIIAVLVVLGAAIVTGAVVAFVMKGSG

SEQ ID NO: 75 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 76 - protein, 134 aa, Artificial Sequence
  Description of Artificial Sequence Synthetic polypeptide
MYSMQLASCVTLTLVLLVNSNWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 77 - protein, 149 aa, Artificial Sequence
  Description of Artificial Sequence Synthetic polypeptide
MYSMQLASCVTLTLVLLVNSHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 78 - protein, 67 aa, Artificial Sequence
  Description of Artificial Sequence Synthetic polypeptide
GGGGSGGGSALGDYKDDDDKLEIRAAFLRQRNTALRTEVAELEQEVQRLENEVSQYETRYGPLGGGK

SEQ ID NO: 79 - protein, 20 aa, Equine rhinitis A virus
QCTNYALLKLAGDVESNPGP

SEQ ID NO: 80 - protein, 22 aa, Foot-and-mouth disease virus
VKQTLNFDLLKLAGDVESNPGP

SEQ ID NO: 81 - protein, 19 aa, Porcine teschovirus 1
ATNFSLLKQAGDVEENPGP

SEQ ID NO: 82 - protein, 18 aa, Thosea asigna virus
EGRGSLLTCGDVEENPGP

SEQ ID NO: 83 - protein, 8 aa, Artificial Sequence
  Description of Artificial Sequence Synthetic peptide
DYKDDDDK

SEQ ID NO: 84 - protein, 10 aa, Artificial Sequence
  Description of Artificial Sequence Synthetic peptide
EQKLISEEDL

SEQ ID NO: 85 - protein, 39 aa, Artificial Sequence
  Description of Artificial Sequence Synthetic polypeptide
SAWSHPQFEKGGGSGGGSGGSAWSHPQFEKGGGGSGGGS

SEQ ID NO: 86 - protein, 23 aa, Artificial Sequence
  Description of Artificial Sequence Synthetic peptide
GKPIPNPLLGLDSTGGGGSGGGS

SEQ ID NO: 87 - protein, 20 aa, Mus musculus
METDTLLLWVLLLWVPGSTG

SEQ ID NO: 88 - protein, 27 aa, Mus musculus
MASPLTRFLSLNLLLLGESIILGSGEA

SEQ ID NO: 89 - protein, 20 aa, Mus musculus
MYSMQLASCVTLTLVLLVNS

SEQ ID NO: 90 - protein, 26 aa, Mus musculus
MVLASSTTSIHTMLLLLLMLFHLGLQ

SEQ ID NO: 91 - protein, 25 aa, Mus musculus
MFHVSFRYIFGIPPLILVLLPVTSS

SEQ ID NO: 92 - protein, 17 aa, Mus musculus
MERTLVCLVVIFLGTVA

SEQ ID NO: 93 - protein, 400 aa, Artificial Sequence
  Description of Artificial Sequence Synthetic polypeptide
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSVD

SEQ ID NO: 94 - protein, 233 aa, Artificial Sequence
  Description of Artificial Sequence Synthetic polypeptide
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 95 - protein, 166 aa, Mus musculus
MVLASSTTSIHTMLLLLLMLFHLGLQASISGRDTHRLTRTLNCSSIVKEIIGKLPEPELKTDDEGPSLRNKSFRRVNLSKFVESQGEVDPEDRYVIKSNLQKLNCCLPTSANDSALPGVFIRDLDDFRKKLRFYMVHLNDLETVLTSRPPQPASGSVSPNRGTVEC

SEQ ID NO: 96 - protein, 192 aa, Mus musculus
MAAMSEDSCVNFKEMMFIDNTLYFIPEENGDLESDNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 97 - DNA, 141 bp, Artificial Sequence
  Description of Artificial Sequence Synthetic polynucleotide
ctggagatccgcgctgcatttttgcgacaaagaaataccgctttgcgaaccgaagtggccgagttggagcaggaagtacaacgcctggaaaatgaagtcagccaatacgaaactcgatatggacctctcggaggtggaaag

SEQ ID NO: 98 - protein, 47 aa, Artificial Sequence
  Description of Artificial Sequence Synthetic polypeptide
LEIEAAFLRQRNTALRTEVAELEQEVQRLENEVSQYETRYGPLGGGK

SEQ ID NO: 99 - protein, 47 aa, Artificial Sequence
  Description of Artificial Sequence Synthetic polypeptide
LEIRAAFLRQRNTALRTRVAELEQEVQRLENEVSQYETRYGPLGGGK

SEQ ID NO: 100 - DNA, 141 bp, Artificial Sequence
  Description of Artificial Sequence Synthetic polynucleotide
ctggagatcgaagctgcatttttgcgacaaagaaataccgctttgcgaaccgaagtggccgagttggagcaggaagtacaacgcctggaaaatgaagtcagccaatacgaaactcgatatggacctctcggaggtggaaag

SEQ ID NO: 101 - DNA, 141 bp, Artificial Sequence
  Description of Artificial Sequence Synthetic polynucleotide
ctggagatccgcgctgcatttttgcgacaaagaaataccgctttgcgaacccgcgtggccgagttggagcaggaagtacaacgcctggaaaatgaagtcagccaatacgaaactcgatatggacctctcggaggtggaaag

SEQ ID NO: 102 - protein, 47 aa, Artificial Sequence
  Description of Artificial Sequence Synthetic polypeptide
LEIEAAFLRQENTALRTEVAELEQEVQRLENEVSQYETRYGPLGGGK

SEQ ID NO: 103 - protein, 47 aa, Artificial Sequence
  Description of Artificial Sequence Synthetic polypeptide
LEIRAAFLRQRNTALRTRVAELEQRVQRLENEVSQYETRYGPLGGGK

SEQ ID NO: 104 - DNA, 141 bp, Artificial Sequence
  Description of Artificial Sequence Synthetic polynucleotide
ctggagatcgaagctgcatttttgcgacaagaaaataccgctttgcgaaccgaagtggccgagttggagcaggaagtacaacgcctggaaaatgaagtcagccaatacgaaactcgatatggacctctcggaggtggaaag

SEQ ID NO: 105 - DNA, 141 bp, Artificial Sequence
  Description of Artificial Sequence Synthetic polynucleotide
ctggagatccgcgctgcatttttgcgacaaagaaataccgctttgcgaacccgcgtggccgagttggagcagcgcgtacaacgcctggaaaatgaagtcagccaatacgaaactcgatatggacctctcggaggtggaaag

SEQ ID NO: 106 - protein, 47 aa, Artificial Sequence
  Description of Artificial Sequence Synthetic polypeptide
LEIEAAFLRQENTALRTRVAELEQEVQRLENEVSQYETRYGPLGGGK

SEQ ID NO: 107 - protein, 47 aa, Artificial Sequence
  Description of Artificial Sequence Synthetic polypeptide
LEIRAAFLRQRNTALRTRVAELEQRVQRLENRVSQYETRYGPLGGGK

SEQ ID NO: 108 - DNA, 141 bp, Artificial Sequence
  Description of Artificial Sequence Synthetic polynucleotide
ctggagatcgaagctgcatttttgcgacaagaaaataccgctttgcgaacccgagtggccgagttggagcaggaagtacaacgcctggaaaatgaagtcagccaatacgaaactcgatatggacctctcggaggtggaaag

SEQ ID NO: 109 - DNA, 141 bp, Artificial Sequence
  Description of Artificial Sequence Synthetic polynucleotide
ctggagatccgcgctgcatttttgcgacaaagaaataccgctttgcgaacccgagtggccgagttggagcagcgagtacaacgcctggaaaatcgagtcagccaatacgaaactcgatatggacctctcggaggtggaaag

SEQ ID NO: 110 - DNA, 141 bp, Artificial Sequence
  Description of Artificial Sequence Synthetic polynucleotide
ctggagattgaggctgcatttcttgaaagagaaaacactgctcttgagacaagagtcgccgaactgaggcagcgcgttcagcgcctgcggaaccgagtatctcaatacaggactcggtacggaccactggggggcggtaag

SEQ ID NO: 111 - protein, 269 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 112 - protein, 254 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 113 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 114 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 115 - protein, 145 aa, Mus musculus
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 116 - protein, 9 aa, Artificial Sequence
  Description of Artificial Sequence Synthetic peptide
CPYSNPSLC

SEQ ID NO: 117 - protein, 16 aa, Artificial Sequence
  Description of Artificial Sequence Synthetic peptide
ELPTQGTFSNVSTNVS

SEQ ID NO: 118 - protein, 9 aa, Artificial Sequence
  Description of Artificial Sequence Synthetic peptide
GGGGSGGGS

SEQ ID NO: 119 - protein, 41 aa, Artificial Sequence
  Description of Artificial Sequence Synthetic polypeptide
ELPTQGTFSNVSTNVSGGGGSGGGSELPTQGTFSNVSTNVS

SEQ ID NO: 120 - protein, 13 aa, Artificial Sequence
  Description of Artificial Sequence Synthetic peptide
SGGGGSSGGGGSD

SEQ ID NO: 121 - protein, 31 aa, Artificial Sequence
  Description of Artificial Sequence Synthetic polypeptide
CPYSNPSLCSGGGGSSGGGGSDCPYSNPSLC

SEQ ID NO: 122 - protein, 7 aa, Homo sapiens
PAKPTTT

SEQ ID NO: 123 - protein, 6 aa, Artificial Sequence
  Description of Artificial Sequence Synthetic peptide
SGGGGS

SEQ ID NO: 124 - protein, 48 aa, Artificial Sequence
  Description of Artificial Sequence Synthetic polypeptide
CPYSNPSLCSGGGGSELPTQGTFSNVSTNVSPAKPTTTACPYSNPSLC

SEQ ID NO: 125 - protein, 457 aa, Mus musculus
MCRAISLRRLLLLLLQLSQLLAVTQGKTLVLGKEGESAELPCESSQKKITVFTWKFSDQRKILGQHGKGVLIRGGSPSQFDRFDSKKGAWEKGSFPLIINKLKMEDSQTYICELENRKEEVELWVFKVTFSPGTSLLQGQSLTLTLDSNSKVSNPLTECKHKKGKVVSGSKVLSMSNLRVQDSDFWNCTVTLDQKKNWFGMTLSVLGFQSTAITAYKSEGESAEFSFPLNFAEENGWGELMWKAEKDSFFQPWISFSIKNKEVSVQKSTKDLKLQLKETLPLTLKIPQVSLQFAGSGNLTLTLDKGTLHQEVNLVVMKVAQLNNTLTCEVMGPTSPKMRLTLKQENQEARVSEEQKVVQVVAPETGLWQCLLSEGDKVKMDSRIQVLSRGVNQTVFLACVLGGSFGFLGFLGLCILCCVRCRHQQRQAARMSQIKRLLSEKKTCQCPHRMQKSHNLI

SEQ ID NO: 126 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 127 - protein, 69 aa, Mus musculus
KSMLTVSNSCCLNTLKKELPLKFIQCYRKMGSSCPDPPAVVFRLNKGRESCASTNKTWVQNHLKKVNPC

SEQ ID NO: 128 - protein, 73 aa, Homo sapiens
KSMQVPFSRCCFSFAEQEIPLRAILCYRNTSSICSNEGLIFKLKRGKEACALDTVGWVQRHRKMLRHCPSKRK

SEQ ID NO: 129 - protein, 70 aa, Mus musculus
ARATNVGRECCLDYFKGAIPIRKLVSWYKTSVECSRDAIVFLTVQGKLICADPKDKHVKKAIRLVKNPRP

SEQ ID NO: 130 - protein, 71 aa, Homo sapiens
ARGTNVGRECCLEYFKGAIPLRKLKTWYQTSEDCSRDAIVFVTVQGRAICSDPNNKRVKNAVKYLQSLERS

SEQ ID NO: 131 - protein, 69 aa, Homo sapiens
AQVGTNKELCCLVYTSWQIPQKFIVDYSETSPQCPKPGVILLTKRGRQICADPNKKWVQKYISDLKLNA

SEQ ID NO: 132 - protein, 68 aa, Mus musculus
GPYGANVEDSICCQDYIRHPLPSRLVKEFFWTSKSCRKPGVVLITVKNRDICADPRQVWVKKLLHKLS

SEQ ID NO: 133 - protein, 69 aa, Homo sapiens
GPYGANMEDSVCCRDYVRYRLPLRVVKHFYWTSDSCPRPGVVLLTFRDKEICADPRVPWVKMILNKLSQ

SEQ ID NO: 134 - protein, 127 aa, Artificial Sequence
  Description of Artificial Sequence Synthetic polypeptide
METDTLLLWVLLLWVPGSTGCPYSNPSLCSGGGGSELPTQGTFSNVSTNVSPAKPTTTACPYSNPSLCGGGGSGGGSALGLEIRAAFLRQRNTALRTEVAELEQEVQRLENEVSQYETRYGPLGGGK

SEQ ID NO: 135 - DNA, 381 bp, Artificial Sequence
  Description of Artificial Sequence Synthetic polynucleotide
atggagacagacacactcctgctatgggtgctgctgctctgggttccaggttccacaggttgcccctattcaaacccatcactgtgcagcggcggcggcggcagcgagctgcctacccagggcaccttcagcaacgtgagcaccaacgtgagccctgccaagcctaccaccaccgcctgcccttacagcaaccctagcctgtgcggtggcggaggaagtggcggaggaagcgccctagggctggagatccgcgctgcatttttgcgacaaagaaataccgctttgcgaaccgaagtggccgagttggagcaggaagtacaacgcctggaaaatgaagtcagccaatacgaaactcgatatggacctctcggaggtggaaag

SEQ ID NO: 136 - protein, 123 aa, Artificial Sequence
  Description of Artificial Sequence Synthetic polypeptide
METDTLLLWVLLLWVPGSTGGSGELPTQGTFSNVSTNVSGGGGSGGGSELPTQGTFSNVSTNVSGGGGSGGGSALGLEIRAAFLRQRNTALRTEVAELEQEVQRLENEVSQYETRYGPLGGGK

SEQ ID NO: 137 - DNA, 369 bp, Artificial Sequence
  Description of Artificial Sequence Synthetic polynucleotide
atggagacagacacactcctgctatgggtgctgctgctctgggttccaggttccacaggtgggtctggggagttgcccactcagggcacattttctaatgtctccactaacgtaagcggaggaggaggaagtggaggaggaagtgagctgcctacccagggcaccttcagcaacgtgagcaccaacgtgagcggtggtggtggtagtggtggtggtagcgccctagggctggagatccgcgctgcatttttgcgacaaagaaataccgctttgcgaaccgaagtggccgagttggagcaggaagtacaacgcctggaaaatgaagtcagccaatacgaaactcgatatggacctctcggaggtggaaag

SEQ ID NO: 138 - protein, 178 aa, Artificial Sequence
  Description of Artificial Sequence Synthetic polypeptide
METDTLLLWVLLLWVPGSTGCPYSNPSLCSGGGGSSGGGGSDCPYSNPSLCGGGGSGGGSLEIEAAFLERENTALETRVAELRQRVQRLRNRVSQYRTRYGPLGGGKGSGCHTQSSPKLEFTFWALVVVAGVLFCYGLLVTVALCVIWTRAKFSRSAETAANLQDTFDALHMQTLAPR

SEQ ID NO: 139 - DNA, 534 bp, Artificial Sequence
  Description of Artificial Sequence Synthetic polynucleotide
atggagacagacacactcctgctatgggtgctgctgctctgggttccaggttccacaggttgcccctattcaaacccatcactgtgcagcggcggcggcggcagcagcggcgggggtggcagcgactgcccttacagcaaccctagcctgtgcggtggcggaggaagtggcggaggaagcctggagattgaggctgcatttcttgaaagagaaaacactgctcttgagacaagagtcgccgaactgaggcagcgcgttcagcgcctgcggaaccgagtatctcaatacaggactcggtacggaccactggggggcggtaagggatctggttgtcatactcagtcatctcctaagctggaattcaccttttgggcactggtcgtggttgctggagtcctgttttgttatggcttgctagtgacagtggctctttgtgttatctggacaagagctaagttcagcagatcagccgagactgccgcaaaccttcaggatacatttgatgctttgcacatgcaaaccctcgcaccacgc

SEQ ID NO: 140 - protein, 391 aa, Artificial Sequence
  Description of Artificial Sequence Synthetic polypeptide
METDTLLLWVLLLWVPGSTGCPYSNPSLCSGGGGSSGGGGSDCPYSNPSLCGGGGSGGGSLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVFLACVLGGSFGFLGFLGLCILCRAKFSRSAETAANLQDTFDALHMQTLAPR

SEQ ID NO: 141 - DNA, 1173 bp, Artificial Sequence
  Description of Artificial Sequence Synthetic polynucleotide
atggagacagacacactcctgctatgggtgctgctgctctgggttccaggttccacaggttgcccctattcaaacccatcactgtgcagcggcggcggcggcagcagcggcgggggtggcagcgactgcccttacagcaaccctagcctgtgcggtggcggaggaagtggcggaggaagcctggagatccgcgctgcatttttgcgacaaagaaataccgctttgcgaaccgaagtggccgagttggagcaggaagtacaacgcctggaaaatgaagtcagccaatacgaaactcgatatggacctctcggaggtggaaagggtggaggtggatcaggtgggggtggatctggtggcggtggatctggaggaggatcgctggagattgaggctgcatttcttgaaagagaaaacactgctcttgagacaagagtcgccgaactgaggcagcgcgttcagcgcctgcggaaccgagtatctcaatacaggactcggtacggaccactggggggcggtaaggaattcaccggcagcaccagcggcagcggcaagcccggcagcggcgagggcagcaccaagggcctagaggtccccaatgggccctggaggtccctcaccttctacccagcctggctcacagtgtcagagggagcaaatgccaccttcacctgcagcttgtccaactggtcggaggatcttatgctgaactggaaccgcctgagtcccagcaaccagactgaaaaacaggccgccttctgtaatggtttgagccaacccgtccaggatgcccgcttccagatcatacagctgcccaacaggcatgacttccacatgaacatccttgacacacggcgcaatgacagtggcatctacctctgtggggccatctccctgcaccccaaggcaaaaatcgaggagagccctggagcagagctagtggtaacagagagaatcctggagacctcaacaagatatcccagcccctcgcccaaaccagaaggccggtttcaaggcatggtgttcctggcctgcgtgctgggcggcagcttcggcttcctgggcttcctgggcctgtgcatcctgtgcagagctaagttcagcagatcagccgagactgccgcaaaccttcaggatacatttgatgctttgcacatgcaaaccctcgcaccacgc

SEQ ID NO: 142 - protein, 423 aa, Artificial Sequence
  Description of Artificial Sequence Synthetic polypeptide
METDTLLLWVLLLWVPGSTGLEIRAAFLRQRNTALRTEVAELEQEVQRLENEVSQYETRYGPLGGGKGGGGSGGGGSGGGGSGGGSLEIEAAFLERENTALETRVAELRQRVQRLRNRVSQYRTRYGPLGGGKEFTGSTSGSGKPGSGEGSTKGVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 143 - DNA, 1269 bp, Artificial Sequence
  Description of Artificial Sequence Synthetic polynucleotide
atggagacagacacactcctgctatgggtgctgctgctctgggttccaggttccacaggtctggagatccgcgctgcatttttgcgacaaagaaataccgctttgcgaaccgaagtggccgagttggagcaggaagtacaacgcctggaaaatgaagtcagccaatacgaaactcgatatggacctctcggaggtggaaagggtggaggtggatcaggtggaggtggatctggtggaggtggatctggaggaggatcgctggagattgaggctgcatttcttgaaagagaaaacactgctcttgagacaagagtcgccgaactgaggcagcgcgttcagcgcctgcggaaccgagtatctcaatacaggactcggtacggaccactggggggcggtaaggaattcaccggcagcaccagcggcagcggcaagcccggcagcggcgagggcagcaccaagggcgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaagatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttcctt

SEQ ID NO: 144 - protein, 377 aa, Artificial Sequence
  Description of Artificial Sequence Synthetic polypeptide
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWEEFTGSTSGSGKPGSGEGSTKGLEIEAAFLERENTALETRVAELRQRVQRLRNRVSQYRTRYGPLGGGK

SEQ ID NO: 145 - DNA, 1131 bp, Artificial Sequence
  Description of Artificial Sequence Synthetic polynucleotide
atggaccagcacacacttgatgtggaggataccgcggatgccagacatccagcaggtacttcgtgcccctcggatgcggcgctcctcagagataccgggctcctcgcggacgctgcgctcctctcagatactgtgcgccccacaaatgccgcgctccccacggatgctgcctaccctgcggttaatgttcgggatcgcgaggccgcgtggccgcctgcactgaacttctgttcccgccacccaaagctctatggcctagtcgctttggtcttgctgcttctgatcgccgcctgtgttcctatcttcacccgcaccgagcctcggccagcgctcacaatcaccacctcgcccaacctgggtacccgagagaataatgcagaccaggtcacccctgtttcccacattggctgccccaacactacacaacagggctctcctgtgttcgccaagctactggctaaaaaccaagcatcgttgtgcaatacaactctgaactggcacagccaagatggagctgggagctcatacctatctcaaggtctgaggtacgaagaagacaaaaaggagttggtggtagacagtcccgggctctactacgtatttttggaactgaagctcagtccaacattcacaaacacaggccacaaggtgcagggctgggtctctcttgttttgcaagcaaagcctcaggtagatgactttgacaacttggccctgacagtggaactgttcccttgctccatggagaacaagttagtggaccgttcctggagtcaactgttgctcctgaaggctggccaccgcctcagtgtgggtctgagggcttatctgcatggagcccaggatgcatacagagactgggagctgtcttatcccaacaccaccagctttggactctttcttgtgaaacccgacaacccatgggaagaattcaccggcagcaccagcggcagcggcaagcccggcagcggcgagggcagcaccaagggcctggagattgaggctgcatttcttgaaagagaaaacactgctcttgagacaagagtcgccgaactgaggcagcgcgttcagcgcctgcggaaccgagtatctcaatacaggactcggtacggaccactggggggcggtaag

SEQ ID NO: 146 - protein, 12 aa, Artificial Sequence
  Description of Artificial Sequence Synthetic peptide
GGGGSSGGGGSD

SEQ ID NO: 147 - protein, 18 aa, Artificial Sequence
  Description of Artificial Sequence Synthetic peptide
GSTSGSGKPGSGEGSTKG

SEQ ID NO: 148 - protein, 21 aa, Artificial Sequence
  Description of Artificial Sequence Synthetic peptide
EFTGSTSGSGKPGSGEGSTKG

SEQ ID NO: 149 - protein, 12 aa, Artificial Sequence
  Description of Artificial Sequence Synthetic peptide
GGGGSGGGSALG

SEQ ID NO: 150 - protein, 4 aa, Homo sapiens
KRKK

SEQ ID NO: 151 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 152 - protein, 27 aa, Unknown
  Description of Unknown CD28 sequence
FWVLVVVGGVLACYSLLVTVAFIIFWV

(19) United States
(12) Patent Application Publication
(10) Pub. No.: US 2021/0179686 A1
(43) Pub. Date: Jun. 17, 2021

(54) CELL SORTING SYSTEMS AND METHODS OF USE

(71) Applicant: MEMORIAL SLOAN-KETTERING CANCER CENTER, New York, NY (US)

(72) Inventors: Scott E. JAMES, New York, NY (US); Marcel R.M. VAN DEN BRINK, New York, NY (US); Lorenz JAHN, Brooklyn, NY (US)

(73) Assignee: MEMORIAL SLOAN-KETTERING CANCER CENTER, New York, NY (US)

(21) Appl. No.: 17/176,388

(22) Filed: Feb. 16, 2021

Related U.S. Application Data
(63) Continuation of application No. PCT/US2019/046764, filed on Aug. 16, 2019.
(60) Provisional application No. 62/798,206, filed on Jan. 29, 2019.
(60) Provisional application No. 62/765,129, filed on Aug. 16, 2018.

(51) Int. Cl. C07K 14/705; G01N 33/569; C07K 14/54

(52) U.S. Cl. CPC C07K 14/705; G01N 33/56972; C07K 14/70517; C07K 14/70521; C07K 14/5418; C07K 14/5443; C07K 2319/22; C07K 14/70596; C07K 2319/03; C07K 2319/73; C07K 2319/43; C07K 2319/41; C07K 14/54

(57) ABSTRACT

The presently disclosed subject matter provides methods and systems for isolating cells expressing specific constructs. In certain non-limiting embodiments, the system comprises a membrane-bound polypeptide and a soluble polypeptide that is capable of dimerizing with the membrane-bound polypeptide.

### CROSS-REFERENCE TO RELATED APPLICATIONS

This application is a Continuation of International Patent Application No. PCT/US19/46764, filed Aug. 16, 2019, which claims priority to U.S. Provisional Application No. 62/765,129, filed on Aug. 16, 2018, and U.S. Provisional Application No. 62/798,206, filed Jan. 29, 2019, the contents of each of which are incorporated by reference in their entirety, and to each of which priority is claimed.

### SEQUENCE LISTING

The specification further incorporates by reference the Sequence Listing submitted herewith via EFS on Feb. 16, 2021. Pursuant to 37 C.F.R. § 1.52(e)(5), the Sequence Listing text file, identified as 072734 1204 SL.txt, is 233,170 bytes and was created on Feb. 16, 2021. The Sequence Listing electronically filed herewith, does not extend beyond the scope of the specification and thus does not contain new matter.

### INTRODUCTION

The presently disclosed subject matter provides methods and compositions for isolating cells expressing specific constructs. It relates to systems comprising a membrane-bound polypeptide and a soluble polypeptide and methods of using the same.

### BACKGROUND OF THE INVENTION

Stably integrating large amounts of genetic information into primary T cells represents a limitation of current cellular engineering. Both retroviruses and lentiviruses exhibit a significant decrease in viral titer as the viral vector insert size exceeds the packaging limit of the virus (about 6-8 kb for retroviruses and about 10-12 kb for lentiviruses). Low viral titers result in low transduction efficiency and low copy number integrations per cell, leading to inferior gene construct expression. Therefore, there remains a need for gene expression systems comprising multiple vectors and a need for isolating cells comprising such systems.

### SUMMARY OF THE INVENTION

The presently disclosed subject matter provides membrane-bound polypeptides. The membrane-bound polypeptides can be used for sorting cells.

In certain embodiments, the membrane-bound polypeptide comprises: a) a transmembrane domain, and b) an extracellular domain comprising a first dimerization domain and a second dimerization domain that is capable of dimerizing with the first dimerization domain at a cell surface. In certain embodiments, each of the first and second dimerization domains comprises a leucine zipper domain. In certain embodiments, the first dimerization domain comprises the amino acid sequence set forth in SEQ ID NO: 1, SEQ ID NO: 2, or SEQ ID NO: 106, and the second dimerization domain comprises the amino acid sequence set forth in SEQ ID NO: 1, SEQ ID NO: 2, or SEQ ID NO: 106. In certain embodiments, the extracellular domain further comprises a linker between the first dimerization domain and the second dimerization domain. In certain embodiments, the linker comprises the amino acid sequence set forth in SEQ ID NO: 3.

In certain embodiments, the extracellular domain further comprises a spacer/hinge domain between the first dimerization domain and the transmembrane domain. In certain embodiments, the spacer/hinge domain comprises an epitope recognized by an antibody, wherein binding of the antibody to the epitope mediates depletion of a cell expressing the membrane-bound polypeptide. In certain embodiments, the spacer/hinge domain comprises a Thy1.1 molecule or a truncated EGFR molecule (EGFRt). In certain embodiments, the Thy1.1 molecule comprises or has the amino acid sequence set forth in SEQ ID NO: 68. In certain embodiments, the EGFRt comprises or has the amino acid sequence set forth in SEQ ID NO: 70.

In certain embodiments, the extracellular domain further comprises a co-stimulatory ligand. In certain embodiments, the co-stimulatory ligand is selected from the group consisting of tumor necrosis factor (TNF) family members, immunoglobulin (Ig) superfamily members, and combinations thereof. In certain embodiments, the TNF family member is selected from the group consisting of 4-1BBL, OX40L, CD70, GITRL, CD40L, CD30L, and combination thereof. In certain embodiments, the co-stimulatory ligand is 4-1BBL.

In certain embodiments, the Ig superfamily member is selected from the group consisting of CD80, CD86, ICOSLG, and combination thereof. In certain embodiments, the co-stimulatory ligand is CD80.

In certain embodiments, the extracellular domain further comprises a dominant negative form of a molecule. In certain embodiments, the molecule is selected from the group consisting of inhibitors of immune checkpoint molecules, tumor necrosis factor receptor superfamily (TNFRSF) members, and Transforming growth factor beta (TGFβ) receptors. In certain embodiment, the immune checkpoint molecule is selected from the group consisting of PD-1, CTLA-4, B7-H3 (also known as “CD276”), B7-H4, BTLA, TIM-3, LAG-3, TIGIT, LAIR1, CD200, CD200R, HVEM, 2B4, CD160, Galectin9, and combinations thereof. In certain embodiments, the immune checkpoint molecule is PD-1. In certain embodiments, the TNFRSF member is selected from the group consisting of Fas, a Tumor necrosis factor receptor, OX40, CD40, CD27, CD30, 4-1BB (also known as “CD137”), and combinations thereof. In certain embodiments, the dominant negative receptor comprises an extracellular domain of TGFβRII or a fragment thereof.

In certain embodiments, the membrane-bound polypeptide further comprises an intracellular domain. In certain embodiments, the intracellular domain comprises a CD3-ζ domain, a costimulatory domain, a suicide gene, or a combination thereof.

In certain embodiments, the membrane-bound polypeptide is expressed from a vector.

The presently disclosed subject matter further provides systems for isolating a cell comprising at least two expression vectors.

In certain embodiments, the at least two expression vectors comprise: a) a membrane-bound polypeptide disclosed herein encoded by a first expression vector, and b) a soluble polypeptide encoded by a second expression vector, comprising a tag and a third dimerization domain that is capable of dimerizing with the first dimerization domain. In certain embodiments, the third dimerization domain dimerizes with the first dimerization domain prior to the dimerization between the first dimerization domain and the second dimerization domain. In certain embodiments, the third dimerization domain dimerizes with the first dimerization domain in the endoplasmic reticulum. In certain embodiments, the soluble polypeptide and the membrane-bound polypeptide are capable of forming a dimer when expressed from the same cell. In certain embodiments, the soluble polypeptide and the membrane-bound polypeptide are capable of forming a dimer in the endoplasmic reticulum, when expressed from the same cell. In certain embodiments, the soluble polypeptide and the membrane-bound polypeptide are not capable of forming a dimer when expressed from different cells due to the dimerization between the first multimerization domain and the second dimerization domain. In certain embodiments, the third dimerization domain comprises the amino acid sequence set forth in SEQ ID NO: 1, SEQ ID NO: 2, or SEQ ID NO: 106.

In certain embodiments, the tag comprises an epitope tag recognized by a first antibody. In certain embodiments, the epitope tag is selected from the group consisting of a Myc-tag, a HA-tag, a Flag-tag, a V5-tag, a T7 tag, a CD34-tag, and combinations thereof. In certain embodiments, the tag comprises an affinity tag that binds to a substrate. In certain embodiments, the affinity tag is selected from the group consisting of a His-tag, a Strep-tag, an E-tag, a streptavidin binding protein tag (SBP-tag), and combinations thereof.

In certain embodiments, the tag further comprises a mimotope recognized by a second antibody. In certain embodiment, binding of the second antibody to the mimotope mediates depletion of a cell comprising the membrane-bound polypeptide. In certain embodiments, the mimotope is a CD20 mimotope and the second antibody is an anti-CD20 antibody. In certain embodiments, the anti-CD20 antibody is Rituximab.

In certain embodiments, the soluble polypeptide further comprises an antigen binding domain. In certain embodiments, the antigen biding domain comprises a single-chain variable fragment (scFv), a soluble ligand, a cytokine, a non-scFv-based antigen recognition motif, or a combination thereof.

In certain embodiments, the soluble polypeptide further comprises a cytokine or a chemokine. In certain embodiments, the cytokine is selected from the group consisting of IL-1, IL-2, IL-3, IL-7, IL-10, IL-12, IL-15, IL-17, IL-18, IL-21, IL-22, IL-36, and combinations thereof. In certain embodiments, the chemokine is selected from the group consisting of CCL1, CCL8, CCL16, CCL17, CCL18, CCL22, and combinations thereof. In certain embodiments, the membrane-bound polypeptide is expressed from a first vector. In certain embodiments, the soluble polypeptide is expressed from a second vector. The first vector can be the same as the second vector. In certain embodiments, the first vector is the same as the second vector, for example, the vector backbone for the first and the second vectors can be the same, while the polypeptide or protein encoded/expressed by the first and second vectors can be different.

In certain embodiments, the at least two expression vectors comprise: a) a membrane-bound polypeptide encoded by a first expression vector, comprising a transmembrane domain and an extracellular domain, wherein the extracellular domain comprises a first dimerization domain and a blocking spacer, and b) a soluble polypeptide encoded by a second expression vector, comprising a tag and a second dimerization domain, wherein each of the first and second dimerization domains comprises a leucine zipper domain, and wherein the blocking spacer prevents dimerization of the membrane-bound polypeptide with the soluble polypeptide when the membrane-bound polypeptide and the soluble polypeptide are not expressed from the same cell.

In certain embodiments, the first dimerization domain comprises the amino acid sequence set forth in SEQ ID NO: 1, SEQ ID NO: 2, or SEQ ID NO: 106, and the second dimerization domain comprises the amino acid sequence set forth in SEQ ID NO: 1, SEQ ID NO: 2, or SEQ ID NO: 106.

In certain embodiments, the blocking spacer is no more than about 25 amino acid residues. In certain embodiments, the blocking spacer is between about 5 amino acid residues and about 25 amino acid residues. In certain embodiments, the blocking spacer is a truncated CD28 spacer or an IgG1 hinge.

Furthermore, the presently disclosed subject matter provides methods of isolating a cell comprising at least two expression vectors, and methods of sorting a plurality of cells comprising at least two expression vectors

In certain embodiments, the method of isolating a cell comprising at least two expression vectors comprises: a) expressing in a cell i) a membrane-bound polypeptide disclosed herein encoded by a first expression vector, and ii) a soluble polypeptide disclosed herein encoded by a second expression vector, comprising a tag and a third dimerization domain that is capable of dimerizing with the first dimerization domain, b) contacting the cell with a substrate that binds to the tag, and isolating the cell that binds to the substrate.

In certain embodiments, the method of sorting a plurality of cells comprising at least two expression vectors comprises: a) transfecting a plurality of cells with i) a first expression vector encoding a membrane-bound polypeptide disclosed herein, and ii) a second vector encoding a soluble polypeptide disclosed herein, e.g., the soluble polypeptide comprising a tag and a third dimerization domain that is capable of dimerizing with the first dimerization domain, b) contacting the cells with a substrate that binds to the tag, and c) isolating one or more cells that bind to the substrate.

In certain embodiments, the third dimerization domain is capable of dimerizing with the first dimerization domain prior to dimerization between the first dimerization domain and the second dimerization domain. In certain embodiments, the third dimerization domain is capable of dimerizing with the first dimerization domain in the endoplasmic reticulum. In certain embodiments, the soluble polypeptide and the membrane-bound polypeptide are capable of forming a dimer when expressed from the same cell. In certain embodiments, the soluble polypeptide and the membrane-bound polypeptide are capable of forming a dimer in the endoplasmic reticulum, when expressed from the same cell. In certain embodiments, the soluble polypeptide and the membrane-bound polypeptide are not capable of forming a dimer when expressed from different cells due to the dimerization between the first dimerization domain and the second dimerization domain. In certain embodiments, step c) is preceded by a step of washing the substrate to remove cells that do not bind to the substrate.

In certain embodiments, the method of isolating a cell comprising at least two expression vectors comprises: a) expressing in a cell i) a membrane-bound polypeptide disclosed herein encoded by a first expression vector, e.g., the membrane-bound polypeptide comprising a transmembrane domain and an extracellular domain, wherein that extracellular domain comprises a first dimerization domain and a blocking spacer, and ii) a soluble polypeptide disclosed herein encoded by a second expression vector, e.g., the soluble polypeptide comprising a tag and a second dimerization domain, wherein both of the first and second dimerization domains comprise a leucine zipper domain, and wherein the blocking spacer prevents dimerization of the membrane-bound polypeptide with the soluble polypeptide when the membrane-bound polypeptide and the soluble polypeptide are not expressed from the same cell, b) contacting the cell with a substrate that binds to the tag, and c) isolating a cell that binds to the substrate.

In certain embodiments, the method of sorting a plurality of cells comprising at least two expression vectors comprises: a) transfecting a plurality of cells with i) a first expression vector encoding a membrane-bound polypeptide disclosed herein, e.g., a the membrane-bound polypeptide comprising a transmembrane domain and an extracellular domain that comprises a first dimerization domain, and ii) a second expression vector encoding a soluble polypeptide disclosed herein, e.g., the soluble polypeptide comprising a tag and a second dimerization domain that is capable of dimerizing with the first dimerization domain, wherein each of the first and second dimerization domains comprises a leucine zipper domain, and wherein the membrane-bound polypeptide does not dimerize with the soluble polypeptide when the membrane-bound polypeptide and the soluble polypeptide are not expressed from the same cell, b) contacting the cells with a substrate that binds to the tag, and c) isolating one or more cells that bind to the substrate.

In certain embodiments, the cell is selected from the group consisting of a T cell, a Natural Killer (NK) cell, a stem cell from which lymphoid cells may be differentiated. In certain embodiments, the cell is a T cell. In certain embodiments, the T cell is selected from the group consisting of a cytotoxic T lymphocyte (CTL), a regulatory T cell, a Natural Killer T (NKT) cell. In certain embodiments, the cell is autologous.

In certain embodiments, the leucine zippers are orthogonal zippers.

The presently disclosed subject matter further provides nucleic acid molecules encoding membrane-bound polypeptides disclosed herein, including vectors comprising such nucleic acid molecules. The presently disclosed subject matter also provides the host cells comprising nucleic acid molecules and vectors disclosed herein. In certain embodiments, the host cell is a T cell. In certain embodiments, the vector is a viral vector. In certain embodiments, the viral vector is a retroviral vector, e.g. a lentiviral vector. In certain embodiments, the vector is a transposon-based vector.

### BRIEF DESCRIPTION OF THE FIGURES

The following Detailed Description, given by way of example but not intended to limit the invention to specific embodiments described, may be understood in conjunction with the accompanying drawings.

FIG. 1A depicts a cell sorting system in accordance with certain embodiments of the presently disclosed subject matter. FIG. 1B depicts that co-transduction with two vectors shown in

FIG. 1A allows magnetic bead sorting of only cells with integration of both viral vectors.

FIG. 2 depicts that under certain circumstances, secreted affinity-tagged leucine zippers can pair with membrane-bound leucine zippers extracellularly.

FIG. 3 depicts that creation of “blocked” membrane-bound leucine zipper leads to favoring of intracellular over extracellular pairing.

FIG. 4 depicts purification of dual-transduced cells intentionally contaminated with non-dual transduced cells.

FIG. 5 depicts purification and testing of dual-transduced cells with iCaspase 9 and CD20-CAR. T cells comprising RR12EE345L-FLAG iCaspase 9 (vector 1) and RR12EE345L-linker-EE12RR345L-Thy1.1 CD20-CAR (vector 2) were incubated with EL4-CD19 and EL4-CD20 targets in the presence or absence of chemical inducer of dimerization (CID).

FIGS. 6A and 6B depict use of a presently disclosed sorting system for purifying multi-functional CART cells. FIG. 6A depicts the leucine zipper sorting system were used to purify cells to above 95%, wherein the cells comprising two vectors expressing CD19-CAR, CD20-CAR and iCaspase9, and optionally IL-18 (upper left panels). These cells were capable of killing CD19 or CD20+ targets (right panels), and incubation with a iCaspase9 dimerizer led to about 90% cell death (lower left panel). FIG. 6B depicts multi-functional CAR T cells specific for CD19 and CD20 and engineered to secrete IL-18. Mouse T cells were co-transduced with vectors encoding (1) FLAG-RR12EE345L leucine zipper, iCaspase9, and a CD19-CAR and (2) linker-blocked RR12EE345L/EE12RR345L Thy1.1 leucine zipper (sort-depletion construct), CD20-CAR+/−IL-18 with intact propeptide (pro-IL-18) or mouse IL-2 signal peptide (sIL-18). The T cells were single-step MACS sorted with anti-FLAG beads to >90% purity and tested for target lysis vs. C1498 CD19, C1498 CD20, and CD1498. IL-18 secretion was assessed by ELISA. Interferon gamma secretion was assessed by cytometric bead array.

FIGS. 7A-7C depict that intrinsically-blocked truncated CD28 membrane proximal hinge-spacer transmembrane leucine zipper facilitated MACS sorting of dual-transduced cell population. FIG. 7A shows C1498 cell line double-transduced with vector 1 (FLAG-RR12EE345L 2A CBR-2A-GFP) and vector 2 (EE12RR345L Myc CD28EC-9C CD2TM 2A Thy1.1). FIG. 7B shows that FLAG staining was limited to the GFP+ BFP+ double transduced population as a result of truncated 9 amino acid hinge-spacer. FIG. 7C shows generation of purified GFP+ BFP+ double transduced population by singe-step anti-FLAG magnetic bead MACS sorting.

FIGS. 8A-8C depict that linker-blocked truncated EGFR-spacer transmembrane leucine zipper facilitated MACS sorting of dual-transduced cell population. FIG. 8A shows C1498 cell line double-transduced with vector 1 (FLAG-RR12EE345L 2A CBR-2A-GFP) and vector 2 (RR12EE345L linker EE12RR345L EGFRt 2A BFP). FIG. 8B shows that FLAG staining was limited to the GFP+ BFP+ double transduced population as a result of linked blocking RR12EE345L leucine zipper. FIG. 8C shows generation of purified GFP+ BFP+ double transduced population by singe-step anti-FLAG magnetic bead MACS sorting.

FIG. 9 depicts double tandem CAR configuration combined with iCaspase9 and Blocked Thy1.1 Leucine Zipper Sort-Suicide Construct. Two retroviral vectors encoding Leucine Zipper Sorting System constructs and tandem CARs were used to transduce T cells. Vector 1 encoded tagged secreted leucine zipper, iCaspase9, and a tandem CAR comprised by CD38 scFv linked to IL-3 cytokine (interchain linker), CD8 hinge, CD8TM, and the CD28zeta signaling motif. Vector 2 encoded blocked Thy1.1 leucine zipper and tandem CAR comprised by CD20 scFv linked to CD19 scFv (interchain linker), CD8 hinge, CD8TM, and CD28zeta signaling motif.

FIGS. 10A and 10B depict that leucine Zipper Sorting System enabled single-step MACS sorting of T cells expressing double tandem CARs. FIG. 10A shows high purity MACS sorting of dual-transduced T cells (left panel). Myc tag staining of tandem CD20-CD19 CAR was weaker than for CD19 single CAR (right panel, compare vs. FIG. 5). However, Thy1.1 was co-expressed on CD20-CD19 CAR vector and showed high co-purification with CD38-IL-3 tandem CAR (middle panel). FIG. 10B shows that single T cell line lysed targets each expressing 1 of 4 separate antigens. Target lysis was determined via detection of residual luciferase activity from the firefly luciferase-transduced C1498 target cells at 24 hours after initiation of the culture.

FIGS. 11A and 11B depict that leucine Zipper Sorting System enabled deletion of sorted T cells using two suicide genes. T cells were transduced with two retroviral vectors encoding tagged secreted leucine zipper sorting construct+tandem CD38-IL-3 CAR+iCaspase9 (vector 1) and tandem CD20-CD19 CAR+blocked Thy1.1 leucine zipper sort-suicide construct (vector 2). Following single-step simultaneous MACS sorting for cells transduced by both vectors with >90% purity, T cells were tested for activity of suicide genes. FIG. 11A shows incubation of sorted or mock-transduced T cells for 40 minutes with anti-Thy1.1 and 10% rabbit complement. Control cells were incubated with media alone. FIG. 11B shows incubation of sorted or mock-transduced T cells for 24 hours in 100 nM of homodimerizer AP20187 or media. In panels A and B, relative survival was calculated as the percentage of viable cells in the treated vs. control cells. Viable T cells were quantified via flow cytometry with CountBright beads and DAPI.

FIGS. 12A-12C depict that truncated EGFR spacer (EGFRt) fused to linker-blocked leucine zipper facilitates cell sorting and antibody dependent cell-mediated cytotoxicity (ADCC). FIG. 12A shows BM185 cell line co-transduced with FLAG-RR12EE345L 2A iCaspase9 and RR12EE345L/EE12RR345L-EGFRt BFP vectors and MACS sorted with anti-FLAG microbeads. FIG. 12B shows incubation of sorted BM185 FLAG-RR iC9|RR/EE-EGFRt or control BM185 (co-expressing firefly luciferase) overnight in media or in 10 nM AP20187 dimerizer to activate iCaspase9. FIG. 12C incubation of sorted BM185 FLAG-RR iC9|RR/EE-EGFRt or control BM185 cells overnight with varying ratios of the effector NK cell line NK92-MI+/−10 ug/mL of cetuximab. In panels FIGS. 12B and C, relative survival was determined after 24 hours via assessing residual luciferase activity compared with untreated cells.

FIG. 13 depicts cytokine-tagged zippers, “zipperkines”, engineered to facilitate secretion and trans-presentation of cytokines while retaining sorting function of affinity-tagged secreted leucine zippers. Cytokines such as IL-7, IL-15, and IL-21 can be fused to affinity tags and a heterodimerizing leucine zipper. Zipperkines can be secreted to interact with cytokine receptors on T cells or co-expressed with intrinsically-blocked transmembrane leucine zipper to facilitate sorting of two vector co-transduced cells and trans-presentation of cytokines.

FIGS. 14A-14C depict that zipperkines retained functional sorting feature of Leucine Zipper Sorting System and promote T cell proliferation. FIG. 14A shows C1498 cell line co-transduced with retroviral vectors encoding (cytokine-RR12EE345L-FLAG 2A BFP) and the intrinsically-blocked transmembrane leucine zipper (EE12RR345L-Myc-CD28EC-9C CD28TM CD3z delta 2A Thy1.1). IL-7, IL-15, and IL-21 zipperkines were detected as trans-presented on the cell surface (FLAG staining) and cells were sorted with anti-FLAG microbeads to obtain highly purified co-transduced cells. FIG. 14B shows primary T cells transduced with IL-15-RR12EE345L-FLAG 2A BFP and EE12RR345L-Myc-CD28EC-9C CD28TM CD3z delta 2A Thy1.1 and sorted with anti-FLAG beads. FLAG staining (top) and BFP (bottom) demonstrate vector 1 expression, while Thy1.1 demonstrated vector 2 expression. FIG. 14C shows incubation of primary T cells with irradiated splenocytes, 0.5 ug/mL anti-CD3, and irradiated sorted C1498 cells trans-presenting IL-7, IL-15, and IL-21 zipperkines or with control C1498 transmembrane leucine zipper-only transduced cells. T cell counts were enumerated 72 hours later by flow cytometry.

FIG. 15 shows a sorting system in accordance with certain embodiments of the presently disclosed subject matter.

FIG. 16 depicts efficient CD34 and CD20 staining by using the IgG1-hinge CD28TM CD3zΔ and CD28-9C CD28TM CD3zΔ capture leucine zippers.

FIG. 17 depicts uniform surface display of circular CD20 mimotope in cells sorted via anti-CD34 magnetic beads.

FIG. 18 depicts selective depletion of double transduced cells using an anti-CD20 antibody.

FIG. 19 depicts selective magnetic sorting and antibody-mediated depletion enabled by separated CD20 and CD34 binding domains.

FIG. 20 depicts the effect of mutant blocking leucine zippers on capture and presentation of secreted leucine zippers.

FIGS. 21A and 21B depict that CD80 (B7-1) molecule functionalized to present blocked capture leucine zipper, permitting magnetic sorting with FLAG-RR12EE345L leucine zipper. FIG. 21A depicts that sorted cells showed high purity for CD19 and CD20 CARs (Myc, Streptag, respectively) and CD80 functionalized leucine zipper. FIG. 21B shows that RR12EE345L linker EE12RR345L CD80 expressing T cells formed conjugates in culture and bound to soluble CD28-Fc.

### DETAILED DESCRIPTION OF THE INVENTION

The presently disclosed subject matter provides membrane-bound polypeptide comprising a transmembrane domain, and an extracellular domain comprising a first dimerization domain and a second dimerization domain that is capable of dimerizing with the first dimerization domain at a cell surface, wherein each of the first and second dimerization domains comprises a leucine zipper. The membrane-bound polypeptides disclosed herein can be used for sorting cells comprising such membrane-bound polypeptides. Furthermore, the presently disclosed subject matter provides systems for isolating cells expressing specific constructs, e.g., a membrane-bound polypeptide disclosed herein. In certain embodiments, the system comprises a membrane-bound polypeptide and a soluble polypeptide, wherein the soluble polypeptide is capable of dimerizing with the membrane-bound polypeptide. In certain embodiments, the soluble polypeptide and the membrane-bound polypeptide are not capable of forming a dimer when expressed from different cells, which enables sorting of cells expressing a specific combination of constructs.

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)2, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). The antibodies of the invention comprise whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant CL region. The light chain constant region is comprised of one domain, CL. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}.

As used herein, a “linker” refers to a functional group (e.g., chemical or polypeptide) that covalently attaches two or more polypeptides or nucleic acids so that they are connected to one another. In certain embodiments, the linker comprises one or more amino acids used to couple two proteins together (e.g., to couple V_{H} and V_{L} domains or to couple two dimerization domains). The linker can be usually rich in glycine for flexibility, as well as serine or threonine for solubility.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

As used herein, the term “expression vector” refers to a recombinant nucleic acid sequence, e.g., a recombinant DNA molecule, containing a desired coding sequence operably linked to appropriate nucleic acid sequences necessary for the expression of the coding sequence in a particular host organism. Nucleic acid sequences necessary for expression in prokaryotes usually include a promoter, an operator (optional), and a ribosome binding site, often along with other sequences. Nucleic acid sequences necessary for expression in eukaryotic cells can include, but are not limited to, promoters, enhancers, and termination and polyadenylation signals.

In certain embodiments, nucleic acid molecules useful in the presently disclosed subject matter include nucleic acid molecules that encode an antibody or an antigen-binding fragment thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule.

As used herein, the term “disease” refers to any condition or disorder that damages or interferes with the normal function of a cell, tissue, or organ. Examples of diseases include neoplasia or pathogenic infection of a cell, tissue, or organ.

An “effective amount” (or “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease (e.g., a neoplasia), or otherwise reduce the pathological consequences of the disease (e.g., a neoplasia). The dose comprising an effective amount is generally determined by the physician on a case-by-case basis and making such a determination is within the level of ordinary skill in the art. Several factors are typically taken into account when determining an appropriate dosage to achieve an effective amount. These factors include age, sex and weight of the subject, the condition being treated, the severity of the condition and the form and effective concentration of the cells (e.g., engineered immune cells) administered.

As used herein, the term “neoplasm” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of skin, bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, spinal cord, spleen, stomach, testes, thymus, thyroid, trachea, urogenital tract, ureter, urethra, uterus, and vagina, or a tissue or cell type thereof. Neoplasia include cancers, such as melanoma, sarcomas, carcinomas, or plasmacytomas (malignant tumor of the plasma cells).

As used herein, the term “immunoresponsive cell” refers to a cell that functions in an immune response, and includes a progenitor of such cell, and a progeny of such cell.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “secreted” refers to a polypeptide that is released from a cell via the secretory pathway through the endoplasmic reticulum, Golgi apparatus, and as a vesicle that transiently fuses at the cell plasma membrane, releasing the proteins outside of the cell.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “subject” refers to any animal (e.g., a mammal), including, but not limited to, humans, non-human primates, rodents, and the like (e.g., which is to be the recipient of a particular treatment).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

In certain non-limiting embodiments, an intracellular signaling domain of a CAR or a ZipR-CAR comprises a CD3ζ polypeptide, which can activate or stimulate a cell (e.g., a cell of the lymphoid lineage, e.g., a T cell). CD3ζ comprises 3 immunoreceptor tyrosine-based activation motifs (ITAMs), and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain non-limiting embodiments, a CAR or a ZipR-CAR can also comprise a spacer/hinge region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and fragments of CD3, a fragment of a CD28 polypeptide, a fragment of a CD8 polypeptide, a variant thereof, or a synthetic spacer sequence.

As used herein, “costimulatory molecules” refer to cell surface molecules other than antigen receptors or their ligands that are required for a response of lymphocytes to antigen. The at least one co-stimulatory signaling region can include a CD28 polypeptide (e.g., intracellular domain of CD28 or a fragment thereof), a 4-1BB polypeptide (e.g., intracellular domain of 4-1BB or a fragment thereof), an OX40 polypeptide (e.g., intracellular domain of OX40 or a fragment thereof), an ICOS polypeptide (e.g., intracellular domain of ICOS or a fragment thereof), a DAP-10 polypeptide (e.g., intracellular domain of DAP10 or a fragment thereof), or a combination thereof. The co-stimulatory molecule can bind to a co-stimulatory ligand. As used herein, the term a “co-stimulatory ligand” refers to a protein expressed on cell surface that upon binding to its receptor produces a co-stimulatory response, i.e., an intracellular response that effects the stimulation provided by an activating signaling domain (e.g., a CD3ζ signaling domain). Non-limiting examples of co-stimulatory ligands include tumor necrosis factor (TNF) family members, immunoglobulin (Ig) superfamily members, or combination thereof the co-stimulatory ligand is selected from the group consisting of tumor necrosis factor (TNF) family members, immunoglobulin (Ig) superfamily members, and combinations thereof. Non-limiting examples of TNF family member include 4-1BBL, OX40L, CD70, GITRL, CD40L, and CD30L. Non-limiting examples of Ig superfamily member include CD80, CD86, and ICOSLG. For example, 4-1BBL may bind to 4-1BB for providing an intracellular signal that in combination with a CAR signal induces an effector cell function of the CAR⁺ T cell. CARs comprising an intracellular signaling domain that comprises a co-stimulatory signaling region comprising a 4-1BB, ICOS or DAP-10 co-stimulatory signaling domain are disclosed in U.S. Pat. No. 7,446,190, which is herein incorporated by reference in its entirety.

As used herein, the term “multimerization” refers to the formation of multimers (including dimers). Multimerization includes dimerization.

As used herein, the term “a conservative sequence modification” refers to an amino acid modification that does not significantly affect or alter the binding characteristics of the presently disclosed polypeptide (e.g., the extracellular antigen-binding domain of the polypeptide) comprising the amino acid sequence. Conservative modifications can include amino acid substitutions, additions and deletions. Modifications can be introduced into the human scFv of the presently disclosed polypeptide by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, the percent homology between two amino acid sequences is equivalent to the percent identity between the two sequences. The percent identity between the two sequences is a function of the number of identical positions shared by the sequences (i.e.,% homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402.

When utilizing BLAST and Gapped BLAST programs, the default parameters of the respective programs (e.g., XBLAST and NBLAST) can be used. 2. Membrane-Bound Polypeptide and Soluble Polypeptides

The presently disclosed subject matter provides systems comprising a membrane-bound polypeptide and a soluble polypeptide, wherein the soluble polypeptide is capable of dimerizing with the membrane-bound polypeptide.

2.1. Membrane-Bound Polypeptide

In certain embodiments, the membrane-bound polypeptide comprises a transmembrane domain and an extracellular domain. In certain embodiments, the membrane-bound polypeptide further comprises an intracellular domain.

2.1.1. Extracellular Domain

In certain embodiments, the extracellular domain of the membrane-bound polypeptide comprises a dimerization domain comprising a leucine zipper domain. In certain embodiments, the dimerization domain is capable of dimerizing with one or more dimerization domain comprised in the membrane-bound polypeptide. In certain embodiments, the dimerization domain is capable of dimerizing with one or more dimerization domain within a soluble polypeptide disclosed herein.

In certain embodiments, the extracellular domain of the membrane-bound polypeptide comprises a first dimerization domain and a second dimerization domain that is capable of dimerizing with the first dimerization domain at a cell surface. In certain embodiments, each of the first and second dimerization domains comprises a leucine zipper domain. In certain embodiments, the first dimerization domain comprises a first leucine zipper domain. In certain embodiments, the second dimerization domain comprises a second leucine zipper domain.

In certain embodiments, the leucine zipper domain comprises a dimerization domain of the Basic-region leucine zipper (bZIP) class of eukaryotic transcription factors. In certain embodiments, the leucine zipper domain comprises a specific alpha helix monomer that can dimerize with anther alpha helix monomer. In certain embodiments, the leucine zipper domain comprises an EE domain that comprises one or more acidic amino acids, e.g., glutamic acid (E). In certain embodiments, the leucine zipper domain comprises an RR domain that comprises one or more basic amino acids, e.g., arginine (R). In certain embodiments, the first leucine zipper domain comprises an RR domain and the second leucine zipper domain comprises an EE domain.

In certain embodiments, the RR domain comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 1 or a fragment thereof. In certain embodiments, the RR domain comprises a modification of SEQ ID NO: 1 or a fragment thereof. In certain embodiments, the modification comprises up to one, up to two, or up to three amino acid substitutions. SEQ ID NO: 1 is provided below.

[SEQ ID NO: 1]
LEIRAAFLRQRNTALRTEVAELEQEVQRLENEVSQYETRYGPL GGGK

An exemplary nucleotide sequence encoding the amino acid sequence of SEQ ID NO: 1 is set forth in SEQ ID NO: 97, which is provided below.

[SEQ ID NO: 97]
CTGGAGATCCGCGCTGCATTTTTGCGACAAAGAAATACCGCTTTGCGAA
CCGAAGTGGCCGAGTTGGAGCAGGAAGTACAACGCCTGGAAAATGAAGT
CAGCCAATACGAAACTCGATATGGACCTCTCGGAGGTGGAAAG

In certain embodiments, the RR domain comprises a modification of SEQ ID NO: 1, wherein the modification consists of or has one amino acid substitution. In certain embodiments, the RR domain comprises the amino acid sequence set forth in SEQ ID NO: 98 or SEQ ID NO: 99. SEQ ID NO: 98 and SEQ ID NO: 99 are provided below.

[SEQ ID NO: 98]
LEIEAAFLRQRNTALRTEVAELEQEVQRLENEVSQYETRYGPLGGGK
[SEQ ID NO: 99]
LEIRAAFLRQRNTALRTRVAELEQEVQRLENEVSQYETRYGPLGGGK

An exemplary nucleotide sequence encoding the amino acid sequence of SEQ ID NO: 98 is set forth in SEQ ID NO: 100, which is provided below.

[SEQ ID NO: 100]
CTGGAGATCGAAGCTGCATTTTTGCGACAAAGAAATACCGCTTTGCGAA
CCGAAGTGGCCGAGTTGGAGCAGGAAGTACAACGCCTGGAAAATGAAGT
CAGCCAATACGAAACTCGATATGGACCTCTCGGAGGTGGAAAG

An exemplary nucleotide sequence encoding the amino acid sequence of SEQ ID NO: 99 is set forth in SEQ ID NO: 101, which is provided below.

[SEQ ID NO: 101]
CTGGAGATCCGCGCTGCATTTTTGCGACAAAGAAATACCGCTTTGCGAA
CCCGCGTGGCCGAGTTGGAGCAGGAAGTACAACGCCTGGAAAATGAAGT
CAGCCAATACGAAACTCGATATGGACCTCTCGGAGGTGGAAAG

In certain embodiments, the RR domain comprises a modification of SEQ ID NO: 1, wherein the modification consists of or has two amino acid substitutions. In certain embodiments, the RR domain comprises the amino acid sequence set forth in SEQ ID NO: 102 or SEQ ID NO: 103. SEQ ID NO: 102 and SEQ ID NO: 103 are provided below.

[SEQ ID NO: 102]
LEIEAAFLRQENTALRTEVAELEQEVQRLENEVSQYETRYGPLGGGK
[SEQ ID NO: 103]
LEIRAAFLRQRNTALRTRVAELEQRVQRLENEVSQYETRYGPLGGGK

An exemplary nucleotide sequence encoding the amino acid sequence of SEQ ID NO: 102 is set forth in SEQ ID NO: 104, which is provided below.

[SEQ ID NO: 104]
CTGGAGATCGAAGCTGCATTTTTGCGACAAGAAAATACCGCTTTGCGAA
CCGAAGTGGCCGAGTTGGAGCAGGAAGTACAACGCCTGGAAAATGAAGT
CAGCCAATACGAAACTCGATATGGACCTCTCGGAGGTGGAAAG

An exemplary nucleotide sequence encoding the amino acid sequence of SEQ ID NO: 103 is set forth in SEQ ID NO: 105, which is provided below.

[SEQ ID NO: 105]
CTGGAGATCCGCGCTGCATTTTTGCGACAAAGAAATACCGCTTTGCGAA
CCCGCGTGGCCGAGTTGGAGCAGCGCGTACAACGCCTGGAAAATGAAGT
CAGCCAATACGAAACTCGATATGGACCTCTCGGAGGTGGAAAG

In certain embodiments, the RR domain comprises a modification of SEQ ID NO: 1, wherein the modification consists of or has three amino acid substitutions. In certain embodiments, the RR domain comprises the amino acid sequence set forth in SEQ ID NO: 106 or SEQ ID NO: 107. SEQ ID NO: 106 and SEQ ID NO: 107 are provided below.

[SEQ ID NO: 106]
LEIEAAFLRQENTALRTRVAELEQEVQRLENEVSQYETRYGPLGGGK
[SEQ ID NO: 107]
LEIRAAFLRQRNTALRTRVAELEQRVQRLENRVSQYETRYGPLGGGK

An exemplary nucleotide sequence encoding the amino acid sequence of SEQ ID NO: 106 is set forth in SEQ ID NO: 108, which is provided below.

[SEQ ID NO: 108]
CTGGAGATCGAAGCTGCATTTTTGCGACAAGAAAATACCGCTTTGCGAAC
CCGAGTGGCCGAGTTGGAGCAGGAAGTACAACGCCTGGAAAATGAAGTCA
GCCAATACGAAACTCGATATGGACCTCTCGGAGGTGGAAAG

An exemplary nucleotide sequence encoding the amino acid sequence of SEQ ID NO: 107 is set forth in SEQ ID NO: 109, which is provided below.

[SEQ ID NO: 109]
CTGGAGATCCGCGCTGCATTTTTGCGACAAAGAAATACCGCTTTGCGAAC
CCGAGTGGCCGAGTTGGAGCAGCGAGTACAACGCCTGGAAAATCGAGTCA
GCCAATACGAAACTCGATATGGACCTCTCGGAGGTGGAAAG

In certain embodiment, the modification is positioned in the “g” residues of the RR domain of the leucine zipper. In certain embodiment, the modification reduces heterodimerization affinity between the membrane-bound polypeptide and a linked soluble polypeptide.

In certain embodiments, the EE domain comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 2 or a fragment thereof. In certain embodiments, the EE domain comprises a modification of SEQ ID NO: 2 or a fragment thereof. In certain embodiments, the modification comprises up to one, up to two, or up to three amino acid substitutions. SEQ ID NO: 2 is provided below.

[SEQ ID NO: 2]
LEIEAAFLERENTALETRVAELRQRVQRLRNRVSQYRTRYGPL GGGK

An exemplary nucleotide sequence encoding the amino acid sequence of SEQ ID NO: 2 is set forth in SEQ ID NO: 110, which is provided below.

[SEQ ID NO: 110]
CTGGAGATTGAGGCTGCATTTCTTGAAAGAGAAAACACTGCTCTTGAGAC
AAGAGTCGCCGAACTGAGGCAGCGCGTTCAGCGCCTGCGGAACCGAGTAT
CTCAATACAGGACTCGGTACGGACCACTGGGGGGCGGTAAG

In certain embodiments, the extracellular domain further comprises a linker between the first dimerization domain and the second dimerization domain. In certain embodiments, the linker comprises the amino acid sequence set forth in SEQ ID NO: 3 or SEQ ID NO: 20. SEQ ID NOs: 3 and 20 are provided below.

[SEQ ID NO: 3]
GGGGSGGGGSGGGGSGGGS
[SEQ ID NO: 20]
GGGGSGGGGSGGGGS

In certain embodiments, a dimerization domain comprises an orthogonal zipper. Orthogonal zippers are coiled coil domains that form heterodimers with their specific partner only and not with other zipper domains. In certain embodiments, orthogonality refers to sets of molecules (e.g., leucine zippers) that are non-cross-reactive, i.e., “orthogonal”, to other sets of molecules. For example, A+B=AB and C+D=CD, but neither A nor B bind to C or D, and vice versa.

In certain embodiments, the first and second leucine zipper domains of the membrane-bound polypeptide are a pair of orthogonal zippers, i.e., the first and the second leucine zipper domains are the specific partners for each other to form heterodimers. Orthogonal zippers include, but are not limited to, RR/EE zippers, Fos/Jun zippers and Fos/synZip zippers. Fos/Jun zippers are previously disclosed in Ransone et al., Genes Dev. 1989 June; 3(6):770-81; Kohler et al., Biochemistry. (2001 Jan); 9; 40(1):130-42, which are incorporated by reference herein. Fos/synZip zippers are previously disclosed in Grigoryan et al., Nature. (2009); 458, 859-864; Reinke et al., J Am Chem Soc. (2010); 132, 6025-6031, which are incorporated by reference herein.

In certain embodiments, the orthogonal zippers comprise an amino acid sequence that is at least about 80%, at least about 85%, at least about 90%, at least about 95%, at least about 96%, at least about 97%, at least about 98%, at least about 99% or at least about 100% homologous or identical to RR/EE zippers, Fos/Jun zippers or Fos/synZip zippers, or a fragment thereof, and/or may comprise up to one, up to two, or up to three amino acid substitutions.

Examples of synZip-9, Fos and Jun zippers are set forth in SEQ ID NOs: 4, 5 and 6, respectively.

[SEQ ID NO: 4]
GSQKVESLKQKIEEL KQRKAQLKNDIANLEKEIAYAET
[SEQ ID NO: 5]
GSELTDTLQAETDQLEDEKSALQTEIANLLKEKEKLEFILAAHR
[SEQ ID NO: 6]
ERISRLEEKVKTLKSQNTELASTASLLREQVAQLKQKVLSHVLE

In certain embodiments, the extracellular domain of the membrane-bound polypeptide further comprises a spacer/hinge domain between a dimerization domain and a transmembrane domain.

In certain embodiments, the spacer/hinge domain can be flexible enough to allow the dimerization domain to orient in different directions to facilitate antigen recognition after dimerizing with a soluble polypeptide disclosed herein. The spacer region can be the hinge region from IgG1, or the CH2CH3 region of immunoglobulin and fragments of CD3, a fragment of a CD28 polypeptide, a fragment of a CD8 polypeptide, a variation of any of the foregoing that is at least about 80%, at least about 85%, at least about 90%, or at least about 95% identical thereto, or a synthetic spacer sequence.

In certain embodiments, the spacer/hinge domain comprises an epitope recognized by an antibody. In certain embodiments, binding of the antibody to the epitope mediates depletion of a cell comprising the membrane-bound polypeptide. In certain embodiments, the spacer/hinge domain comprises a Thy1.1 molecule, a truncated EGFR molecule (EGFRt), CD22 immunoglobulin-like domain epitope, an IgG/Fc domain (can be a Fc from any IgG), CD2, CD20 cyclic mimotope, CD30, CD52, or HER2.

In certain embodiments, the membrane-bound polypeptide further comprises a blocking spacer, wherein the blocking spacer is capable of preventing dimerization of the membrane-bound polypeptide with a soluble polypeptide when the membrane-bound polypeptide and the soluble polypeptide are not expressed from the same cell. In certain embodiments, the blocking spacer comprises a minimum spacer of no more than about 20 to about 30 amino acid residues. In certain embodiments, the blocking spacer comprises no more than about 25 amino acid residues. In certain embodiments, the blocking spacer comprises about 1, about 2, about 3, about 4, about 5, about 6, about 7, about 8, about 9 about 10, about 11, about 12, about 13, about 14, about 15, about 16, about 17, about 18, about 19 or about 20 amino acid residues. In certain embodiments, the blocking spacer comprises between about 5 amino acid residues and about 25 amino acid residues, between about 5 amino acid residues and about 20 amino acid residues, between about 10 amino acid residues and about 25 amino acid residues or between about 10 amino acid residues and about 20 amino acid residues.

In certain embodiments, the blocking spacer comprises an amino acid sequence that is at least about 80%, at least about 85%, at least about 90%, at least about 95%, at least about 96%, at least about 97%, at least about 98%, at least about 99% or at least about 100% homologous or identical to a truncated CD28 spacer set forth in SEQ ID NO: 7 or SEQ ID NO: 21, or a fragment thereof. In certain embodiments, the blocking spacer comprises an amino acid sequence that is at least about 80%, at least about 85%, at least about 90%, at least about 95%, at least about 96%, at least about 97%, at least about 98%, at least about 99% or at least about 100% homologous to an IgG1 hinge set forth in SEQ ID NO: 8 or SEQ ID NO:22, or a fragment thereof. In certain embodiments, the blocking spacer comprises a modification of SEQ ID NO: 7, SEQ ID NO: 8, SEQ ID NO: 21, SEQ ID NO: 22, wherein the modification up to one, up to two, or up to three amino acid substitutions.

[SEQ ID NO: 7]
CHTQSSPKL
[SEQ ID NO: 8]
VPRDCGCKPCICT
[SEQ ID NO: 21]
CPSPLFPGPSKP
[SEQ ID NO: 22
EPKSCDKTHTCPPC]

In certain embodiments, the blocking spacer has a length of no more than about 25 amino acids. In certain embodiments, the blocking spacer has a length of between about 5 amino acids and about 25 amino acids. In certain embodiments, the blocking spacer is a truncated CD28 spacer or an IgG1 hinge.

In certain non-limiting embodiments, the extracellular domain of the membrane-bound polypeptide comprises at least one co-stimulatory ligand or a fragment thereof.

In certain embodiments, the co-stimulatory ligand is selected from the group consisting of tumor necrosis factor (TNF) family members, immunoglobulin (Ig) superfamily members, and combination thereof. In certain embodiments, the co-stimulatory ligand is selected from the group consisting of tumor necrosis factor (TNF) family members, immunoglobulin (Ig) superfamily members, and combinations thereof. In certain embodiments, the TNF family member is selected from the group consisting of 4-1BBL, OX40L, CD70, GITRL, CD40L, and CD30L.

In certain embodiments, the Ig superfamily member is selected from the group consisting of CD80, CD86, and ICOSLG.

In certain embodiments, the co-stimulatory ligand is CD80. In certain embodiments, the CD80 is a mouse CD80. In certain embodiments, the CD80 comprises the amino acid sequence set forth in SEQ ID NO: 111. In certain embodiments, the CD80 is a human CD80. In certain embodiments, the CD80 comprises the amino acid sequence set forth in SEQ ID NO: 112. SEQ ID Nos: 111 and 112 are provided below.

[SEQ ID NO: 111]
VDEQLSKSVKDKVLLPCRYNSPHEDESEDRIYWQKHDKVVLSVIAGKLKV
WPEYKNRTLYDNTTYSLIILGLVLSDRGTYSCVVQKKERGTYEVKHLALV
KLSIKADFSTPNITESGNPSADTKRITCFASGGFPKPRFSWLENGRELPG
INTTISQDPESELYTISSQLDFNTTRNHTIKCLIKYGDAHVSEDFTWEKP
PEDPPDSKNTLVLFGAGFGAVITVVVIVVIIKCFCKHRSCFRRNEASRET
NNSLTFGPEEALAEQTVFL
[SEQ ID NO: 112]
VIHVTKEVKEVATLSCGHNVSVEELAQTRIYWQKEKKMVLTMMSGDMNIW
PEYKNRTIFDITNNLSIVILALRPSDEGTYECVVLKYEKDAFKREHLAEV
TLSVKADEPTPSISDFEIPTSNIRRIICSTSGGFPEPHLSWLENGEELNA
INTTVSQDPETELYAVSSKLDFNMTTNHSFMCLIKYGHLRVNQTFNWNTT
KQEHFPDNLLPSWAITLISVNGIFVICCLTYCFAPRCRERRRNERLRRES
VRPV

In certain embodiments, the co-stimulatory ligand is 4-1BBL. In certain embodiments, the 4-1BBL is a mouse 4-1BBL. In certain embodiments, the 4-1BBL comprises the amino acid sequence set forth in SEQ ID NO: 113. In certain embodiments, the 4-1BBL is a human 4-1BBL. In certain embodiments, the 4-1BBL comprises the amino acid sequence set forth in SEQ ID NO: 114. SEQ ID Nos: 113 and 114 are provided below.

[SEQ ID NO: 113]
MDQHTLDVEDTADARHPAGTSCPSDAALLRDTGLLADAALLSDTVRPTNA
ALPTDAAYPAVNVRDREAAWPPALNFCSRHPKLYGLVALVLLLLIAACVP
IFTRTEPRPALTITTSPNLGTRENNADQVTPVSHIGCPNTTQQGSPVFAK
LLAKNQASLCNTTLNWHSQDGAGSSYLSQGLRYEEDKKELVVDSPGLYYV
FLELKLSPTFTNTGHKVQGWVSLVLQAKPQVDDFDNLALTVELFPCSMEN
KLVDRSWSQLLLLKAGHRLSVGLRAYLHGAQDAYRDWELSYPNTTSFGLF
LVKPDNPWE
[SEQ ID NO: 114]
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

In certain non-limiting embodiments, the extracellular domain of the membrane-bound polypeptide further comprises a dominant negative molecule or a fragment thereof. In certain embodiments, the dominant negative molecules is selected from the group consisting of inhibitors of immune checkpoint molecules, tumor necrosis factor receptor superfamily (TNFRSF) members, and TGFβ receptors. In certain embodiment, the immune checkpoint molecule is selected from the group consisting of PD-1, CTLA-4, B7-H3, B7-H4, BTLA, TIM-3, LAG-3, TIGIT, LAIR1, CD200, CD200R, HVEM, 2B4, CD160, Galectin9, and combinations thereof. In certain embodiments, the immune checkpoint molecule is PD-1. In certain embodiments, the TNFRSF member is selected from the group consisting of Fas, a Tumor necrosis factor receptor, OX40, CD40, CD27, CD30, 4-1BB, and combinations thereof. In certain embodiments, the dominant negative receptor comprises an extracellular domain of TGFβRII or a fragment thereof.

In certain non-limiting embodiments, the dominant negative molecule is an inhibitor of an immune checkpoint molecule. Details of dominant negative (DN) forms of inhibitors of an immune checkpoint molecule are disclosed in WO2017/040945 and WO2017/100428, the contents of each of which are incorporated by reference herein in their entireties. In certain embodiments, the extracellular domain of the membrane-bound polypeptide further comprises a dominant negative form of an immune checkpoint inhibitor disclosed in WO2017/040945. In certain embodiments, the extracellular domain of the membrane-bound polypeptide further comprises a dominant negative form of an immune checkpoint inhibitor disclosed in WO2017/100428.

In certain embodiments, the dominant negative molecule is a PD-1 dominant negative (i.e., PD-1 DN) molecule. In certain embodiments, the PD-1 DN comprises (a) at least a fragment of an extracellular domain of PD-1 comprising a ligand binding region, and (b) a transmembrane domain.

In certain embodiments, the PD-1 DN is a mouse PD-1 DN. In certain embodiments, the PD-1 DN comprises or has the amino acid sequence set forth in SEQ ID NO: 115, which is provided below. In certain embodiments, the PD-1 DN is a human PD-1 DN.

[SEQ ID NO: 115]
LEVPNGPWRSLTFYPAWLTVSEGANATFTCSLSNWSEDLMLNWNRLSPSN
QTEKQAAFCNGLSQPVQDARFQIIQLPNRHDFHMNILDTRRNDSGIYLCG
AISLHPKAKIEESPGAELVVTERILETSTRYPSPSPKPEGRFQGM

In certain embodiments, the extracellular domain of the membrane-bound polypeptide further comprises a tag. In certain embodiments, the tag comprises an epitope tag recognized by a first antibody. Non-limiting examples of epitope tags include Myc-tag, a HA-tag, a Flag-tag, a V5-tag, a T7-tag, and a CD34-tag. In certain embodiments, the epitope tag is a CD34-tag.

In certain embodiments, the tag comprises an affinity tag that binds to a substrate. Non-limiting examples of affinity tags include a His-tag, a Strep-tag, an E-tag, and a streptavidin binding protein tag (SBP-tag).

Furthermore, the extracellular domain of the membrane-bound polypeptide can further comprise a mimotope recognized by a second antibody. Binding of the second antibody to the mimotope can mediates depletion of a cell comprising the membrane-bound polypeptide. In certain embodiments, the mimotope is a CD20 mimotope recognized by an anti-CD20 antibody. In certain embodiments, the anti-CD20 antibody is Rituxumab.

In certain embodiments, the extracellular domain of the membrane-bound polypeptide comprises an epitope tag recognized by a first antibody and a mimotope recognized by a second antibody. In certain embodiments, the epitope tag is a CD34 epitope tag, the first antibody is an anti-CD34 antibody, the mimotope is a CD20 mimotope, and the second antibody is an anti-CD20 antibody. In certain embodiments, the anti-CD34 antibody is QBEND10. In certain embodiments, the anti-CD20 antibody is Rituximab. In certain embodiments, the CD20 mimotope is a circular CD20 mimotope.

In certain embodiments, the CD20 mimotope comprises or has the amino acid sequence set forth in SEQ ID NO: 116, which is provided below.

| | |
|---|---|
| CPYSNPSLC | [SEQ ID NO: 116] |

In certain embodiments, the CD34 epitope tag comprises or has the amino acid sequence set forth in SEQ ID NO: 117, which is provided below.

| | |
|---|---|
| ELPTQGTFSNVSTNVS | [SEQ ID NO: 117] |

In certain embodiments, the extracellular domain of the membrane-bound polypeptide comprises two CD34 epitope tags, e.g., each CD34 epitope tag comprises or has the amino acid sequence set forth in SEQ ID NO: 117. In certain embodiments, the two CD34 epitope tags are connected by a linker. In certain embodiments, the linker comprises or has the amino acid sequence set forth in SEQ ID NO: 118, which is provided below.

| | |
|---|---|
| GGGGSGGGS | [SEQ ID NO: 118] |

In certain embodiments, the extracellular domain of the membrane-bound polypeptide comprises the amino acid sequence set forth in SEQ ID NO: 119, which is provided below. SEQ ID NO: 119 has two CD34 epitope tags, which are linked by the linker having the amino acid sequence set forth in SEQ ID NO: 118, and SEQ ID NO: 119 is referred to “Q2” or “Q2 sequence”.

[SEQ ID NO: 119]
ELPTQGTFSNVSTNVSGGGGSGGGSELPTQGTFSNVSTNVS

In certain embodiments, the extracellular domain of the membrane-bound polypeptide comprises two CD20 mimotopes, e.g., each CD20 mimotope comprises or has the amino acid sequence set forth in SEQ ID NO: 116. In certain embodiments, the two CD20 mimotopes are connected by a linker. In certain embodiments, the linker comprises or has the amino acid sequence set forth in SEQ ID NO: 120, which is provided below.

| | |
|---|---|
| SGGGGSSGGGGSD | [SEQ ID NO: 120] |

In certain embodiments, the extracellular domain of the membrane-bound polypeptide comprises the amino acid sequence set forth in SEQ ID NO: 121, which is provided below. SEQ ID NO: 121 has two CD20 mimotopes, which are linked by the linker having the amino acid sequence set forth in SEQ ID NO: 120, and SEQ ID NO: 121 is referred to “R2” or “R2 sequence”.

| | |
|---|---|
| CPYSNPSLCSGGGGSSGGGGSDCPYSNPSLC | [SEQ ID NO: 121] |

In certain embodiments, the extracellular domain of the membrane-bound polypeptide comprises two CD20 mimotopes and one CD34 epitope tag, e.g., each CD20 mimotope comprises or has the amino acid sequence set forth in SEQ ID NO: 116, and the CD34 epitope tag comprises or has the amino acid sequence set forth in SEQ ID NO: 117. In certain embodiments, the CD34 epitope tag is linked to each CD20 mimotope with a linker. In certain embodiments, the linker is a human CD8 polypeptide, e.g., one comprising or having the amino acid sequence set forth in SEQ ID NO: 122, which is provided below.

| | |
|---|---|
| PAKPTTT | [SEQ ID NO: 122] |

In certain embodiments, the linker comprises or has the amino acid sequence set forth in SEQ ID NO: 123, which is provided below.

| | |
|---|---|
| SGGGGS | [SEQ ID NO: 123] |

In certain embodiments, the extracellular domain of the membrane-bound polypeptide comprises the amino acid sequence set forth in SEQ ID NO: 124, which is provided below. SEQ ID NO: 124 has two CD20 mimotopes and one CD34 epitope tag, wherein, the CD34 epitope tag is linked to one CD20 mimotope with a linker having the amino acid sequence set forth in SEQ ID NO: 122, and linked with another CD20 mimotope with a linker having the amino acid sequence set forth in SEQ ID NO: 123, and SEQ ID NO: 124 is referred to “RQR” or “RQR sequence”.

[SEQ ID NO: 124]
CPYSNPSLCSGGGGSELPTQGTFSNVSTNVSPAKPTTTACPYSNPSLC

2.1.2. Transmembrane Domain

Different transmembrane domains can result in different receptor stabilities. In accordance with the presently disclosed subject matter, the transmembrane domain can comprise a CD8 polypeptide (e.g., the transmembrane domain of CD8 or a fragment thereof), a CD28 polypeptide (e.g., the transmembrane domain of CD28 or a fragment thereof), a CD3ζ polypeptide (e.g., the transmembrane domain of CD3ζ or a fragment thereof), a CD4 polypeptide (e.g., the transmembrane domain of CD4 or a fragment thereof), a 4-1BB polypeptide (e.g., the transmembrane domain of 4-1BB or a fragment thereof), an OX40 polypeptide (e.g., the transmembrane domain of OX40 or a fragment thereof), an ICOS polypeptide (e.g., the transmembrane domain of ICOS or a fragment thereof), a CD2 polypeptide (e.g., the transmembrane domain of CD2 or a fragment thereof), a synthetic peptide (not based on a protein associated with the immune response), or a combination thereof.

In certain embodiments, the transmembrane domain of the membrane-bound polypeptide comprises a CD8 polypeptide (e.g., the transmembrane domain of CD8 or a fragment thereof). In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001139345.1 (SEQ ID NO: 9) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 9 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, 183 to 203, or 200 to 235 of SEQ ID NO: 9. In certain embodiments, the transmembrane domain of the membrane-bound polypeptide comprises a CD8 polypeptide comprising or having an amino acid sequence of amino acids 183 to 203 of SEQ ID NO: 9.

SEQ ID NO: 9 is provided below.

[SEQ ID NO: 9]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: AAA92533.1 (SEQ ID NO: 10) or a fragment thereof, and/or may optionally comprise up to one or up to two or up to three conservative amino acid substitutions. In certain embodiments, the CD8 polypeptide comprises or has an amino acid sequence that is a consecutive fragment of SEQ ID NO: 10 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, or 200 to 247 of SEQ ID NO: 10. SEQ ID NO: 10 is provided below.

[SEQ ID NO: 10]
MASPLTRFLSLNLLLMGESIILGSGEAKPQAPELRIFPKKMDAELGQKVD
LVCEVLGSVSQGCSWLFQNSSSKLPQPTFVVYMASSHNKITWDEKLNSSK
LFSAVRDTNNKYVLTLNKFSKENEGYYFCSVISNSVMYFSSVVPVLQKVN
STTTKPVLRTPSPVHPTGTSQPQRPEDCRPRGSVKGTGLDFACDIYIWAP
LAGICVAPLLSLIITLICYHRSRKRVCKCPRPLVRQEGKPRPSEKIV

In certain embodiments, the CD8 polypeptide comprises or has the amino acid sequence set forth in SEQ ID NO: 11, which is provided below:

[SEQ ID NO: 11]
IYIWAPLAGICVALLLSLIITLICY

In certain embodiments, the CD8 polypeptide comprises or has the amino acid sequence set forth in SEQ ID NO: 12, which is provided below:

| | |
|---|---|
| IYIWAPLAGTCGVLLLSLVIT | [SEQ ID NO: 12] |

In certain embodiments, the transmembrane domain of the membrane-bound polypeptide comprises a CD28 polypeptide (e.g., the transmembrane domain of CD28 or a fragment thereof). The CD28 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P10747 or NP_006130 (SEQ ID No: 14) or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 14 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, 153 to 179, or 200 to 220 of SEQ ID NO: 14. In certain embodiments, the transmembrane domain of a presently disclosed membrane-bound polypeptide comprises a CD28 polypeptide comprising or having an amino acid sequence of amino acids 153 to 179 of SEQ ID NO: 14. SEQ ID NO: 14 is provided below:

[SEQ ID NO: 14]
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In certain embodiments, the transmembrane domain of a membrane-bound polypeptide comprises a CD28 polypeptide comprising or having the amino acid sequence set forth in SEQ ID NO: 152 as provided below.

[SEQ ID NO: 152]
FWVLVVVGGVLACYSLLVTVAFIIFWV

In certain embodiments, the transmembrane domain of a membrane-bound polypeptide comprises a CD28 polypeptide comprising or having the amino acid sequence set forth in SEQ ID NO: 23 as provided below.

[SEQ ID NO: 23]
FWALVVVAGVLFCYGLLVTVALCVIWT

In certain embodiments, the transmembrane domain of the membrane-bound polypeptide comprises a CD4 polypeptide (e.g., the transmembrane domain of CD4 or a fragment thereof). The CD4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_038516.1 (SEQ ID No: 125) or a fragment thereof, and/or may optionally comprise up to one or up to two or up to three conservative amino acid substitutions. In certain embodiments, the CD4 polypeptide comprises or has an amino acid sequence that is a consecutive fragment of SEQ ID NO: 125 which is at least 20, or at least 30, or at least 40, or at least 50, and up to 457 amino acids in length. Alternatively or additionally, in non-limiting various embodiments, the CD4 polypeptide comprises or has an amino acid sequence of amino acids 1 to 457, 1 to 50, 50 to 100, 100 to 150, 150 to 200, 200 to 250, 250 to 300, 300 to 350, 350 to 400, 395 to 417, or 400 to 457 of SEQ ID NO: 125. In certain embodiments, the transmembrane domain of the membrane-bound polypeptide comprises a CD4 polypeptide comprising or having amino acids 395 to 417 of SEQ ID NO: 125. SEQ ID NO: 125 is provided below:

[SEQ ID NO: 125]
MCRAISLRRLLLLLLQLSQLLAVTQGKTLVLGKEGESAELPCESSQKKIT
VFTWKFSDQRKILGQHGKGVLIRGGSPSQFDRFDSKKGAWEKGSFPLIIN
KLKMEDSQTYICELENRKEEVELWVFKVTFSPGTSLLQGQSLTLTLDSNS
KVSNPLTECKHKKGKVVSGSKVLSMSNLRVQDSDFWNCTVTLDQKKNWFG
MTLSVLGFQSTAITAYKSEGESAEFSFPLNFAEENGWGELMWKAEKDSFF
QPWISFSIKNKEVSVQKSTKDLKLQLKETLPLTLKIPQVSLQFAGSGNLT
LTLDKGTLHQEVNLVVMKVAQLNNTLTCEVMGPTSPKMRLTLKQENQEAR
VSEEQKVVQVVAPETGLWQCLLSEGDKVKMDSRIQVLSRGVNQTVFLACV
LGGSFGFLGFLGLCILCCVRCRHQQRQAARMSQIKRLLSEKKTCQCPHRM
QKSHNLI

In certain embodiments, the transmembrane domain of the membrane-bound polypeptide comprises a CD4 polypeptide (e.g., the transmembrane domain of CD4 or a fragment thereof). The CD4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_000607.1 (SEQ ID No: 126) or a fragment thereof, and/or may optionally comprise up to one or up to two or up to three conservative amino acid substitutions. In certain embodiments, the CD4 polypeptide comprises or has an amino acid sequence that is a consecutive fragment of SEQ ID NO: 126 which is at least 20, or at least 30, or at least 40, or at least 50, and up to 458 amino acids in length. Alternatively or additionally, in various embodiments, the CD4 polypeptide comprises or has an amino acid sequence of amino acids 1 to 457, 1 to 50, 50 to 100, 100 to 150, 150 to 200, 200 to 250, 250 to 300, 300 to 350, 350 to 400, 397 to 418, or 400 to 457 of SEQ ID NO: 126. In certain embodiments, the transmembrane domain of the membrane-bound polypeptide comprises a CD4 polypeptide comprising or having amino acids 397 to 418 of SEQ ID NO: 126. SEQ ID NO: 126 is provided below:

[SEQ ID NO: 126]
MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI

2.1.3. Intracellular Domain

In certain non-limiting embodiments, the membrane-bound polypeptide further comprises an intracellular domain. In certain non-limiting embodiments, the intracellular domain provides an activation signal to a cell (e.g., a cell of the lymphoid lineage, e.g., a T cell). In certain embodiments, the intracellular domain of the membrane-bound polypeptide comprises an immune activating molecule. In certain embodiments, the immune activating molecule is a CD3ζ polypeptide.

In certain non-limiting embodiments, the intracellular domain of the membrane-bound polypeptide comprises a CD3ζ polypeptide or a fragment thereof. CD3ζ can activate or stimulate a cell. CD3ζ comprises 3 ITAMs, and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs. In certain embodiments, the CD3ζ polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_932170 (SEQ ID No: 15) or a fragment thereof, and/or may optionally comprise up to one or up to two or up to three conservative amino acid substitutions. In certain non-limiting embodiments, the CD3ζ polypeptide comprises or has an amino acid sequence that is a consecutive fragment of SEQ ID NO: 15, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 52 to 164, 100 to 150, or 150 to 164 of SEQ ID NO: 15. In certain embodiments, the CD3ζ polypeptide comprises or has an amino acid sequence of amino acids 52 to 164 of SEQ ID NO: 15. SEQ ID NO: 15 is provided below:

[SEQ ID NO: 15]
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the CD3ζ polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001106864.2 (SEQ ID No: 13) or a fragment thereof, and/or may optionally comprise up to one or up to two or up to three conservative amino acid substitutions. In certain non-limiting embodiments, the CD3ζ polypeptide comprises or has an amino acid sequence that is a consecutive fragment of SEQ ID NO: 13, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 13. SEQ ID NO: 13 is provided below:

[SEQ ID NO: 13]
MKWKVSVLACILHVRFPGAEAQSFGLLDPKLCYLLDGILFIYGVIITALY
LRAKFSRSAETAANLQDPNQLYNELNLGRREEYDVLEKKRARDPEMGGKQ
RRRNPQEGVYNALQKDKMAEAYSEIGTKGERRRGKGHDGLYQDSHFQAVQ
FGNRREREGSELTRTLGLRARPKACRHKKPLSLPAAVS

In certain embodiments, the CD3ζ polypeptide comprises or has an amino acid sequence set forth in SEQ ID NO: 17 as provided below.

[SEQ ID NO: 17]
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

In certain embodiments, the intracellular domain of the membrane-bound polypeptide comprises a murine CD3ζ polypeptide.

In certain embodiments, the intracellular domain of the membrane-bound polypeptide comprises a human CD3ζ polypeptide.

In certain non-limiting embodiments, the intracellular domain of the membrane-bound polypeptide provides an activation signal and a stimulation signal to a cell. In certain embodiments, the intracellular domain of the membrane-bound polypeptide comprises at least one costimulatory molecule or a fragment thereof.

In certain embodiments, the at least one co-stimulatory signaling region comprises a CD28 polypeptide (e.g., the intracellular domain of CD28 or a fragment thereof), a 4-1BB polypeptide (e.g., the intracellular domain of 4-1BB or a fragment thereof), an OX40 polypeptide (e.g., the intracellular domain of OX40 or a fragment thereof), an ICOS polypeptide (e.g., the intracellular domain of ICOS or a fragment thereof), a DAP-10 polypeptide (e.g., the intracellular domain of DAP-10 or a fragment thereof), or a fragment or a combination thereof. In certain embodiments, the at least one co-stimulatory signaling region comprises a CD28 polypeptide. In certain embodiments, the at least one co-stimulatory signaling region comprises an intracellular domain of CD28 or a fragment thereof.

In certain embodiments, the costimulatory molecule is a CD28 polypeptide. The CD28 polypeptide can comprise or have an amino acid sequence that is at least about 80%, at least about 85%, at least about 90%, at least about 95%, at least about 96%, at least about 97%, at least about 98%, at least about 99% or at least 100% homologous or identical to the sequence having a NCBI Reference No: P10747 or NP_006130 (SEQ ID NO: 14) or a fragment thereof, and/or may optionally comprise up to one or up to two or up to three conservative amino acid substitutions. In certain embodiments, the CD28 polypeptide comprises or has an amino acid sequence that is a consecutive fragment of SEQ ID NO: 14 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 150 to 200, or 200 to 220 of SEQ ID NO: 14. In certain embodiments, the CD28 polypeptide comprises or has an amino acid sequence of amino acids 181 to 220 of SEQ ID NO: 14.

In certain embodiments, the CD28 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_031668.3 (SEQ ID NO: 16), or fragments thereof, and/or may optionally comprise up to one or up to two or up to three conservative amino acid substitutions. In certain embodiments, the CD28 polypeptide comprises or has an amino acid sequence that is a consecutive fragment of SEQ ID NO: 16 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 150 to 200, or 200 to 218 of SEQ ID NO: 16. SEQ ID NO: 16 is provided below:

[SEQ ID NO: 16]
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTFRL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In certain embodiments, the costimulatory molecule is a mouse CD28 polypeptide. In certain embodiments, the costimulatory molecule is a human CD28 polypeptide.

In certain embodiments, the intracellular domain of the membrane-bound polypeptide comprises two costimulatory molecules, e.g., CD28 and 4-1BB or CD28 and OX40.

In certain embodiments, the at least one co-stimulatory signaling region comprises a 4-1BB polypeptide. In certain embodiments, the at least one co-stimulatory signaling region comprises an intracellular domain of 4-1BB or a fragment thereof.

In certain embodiments, the costimulatory molecule is a 4-1BB polypeptide (e.g., the intracellular domain of 4-1BB or a fragment thereof). 4-1BB can act as a tumor necrosis factor (TNF) ligand and have stimulatory activity. The 4-1BB polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P41273 or NP_001552 (SEQ ID NO: 151) or a fragment thereof, and/or may optionally comprise up to one or up to two or up to three conservative amino acid substitutions. SEQ ID NO: 151 is provided below:

[SEQ ID NO: 151]

| | |
|---|---|
| 1 | MGNSCYNIVA TLLLVLNFER TRSLQDPCSN CPAGTFCDNN |
| | RNQICSPCPP NSFSSAGGQR |
| 61 | TCDICRQCKG VFRTRKECSS TSNAECDCTP GFHCLGAGCS |
| | MCEQDCKQGQ ELTKKGCKDC |
| 121 | CFGTFNDQKR GICRPWTNCS LDGKSVLVNG TKERDVVCGP |
| | SPADLSPGAS SVTPPAPARE |
| 181 | PGHSPQIISF FLALTSTALL FLLFFLTLRF SVVKRGRKKL |
| | LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

In certain embodiments, the at least one co-stimulatory signaling region comprises an OX40 polypeptide. In certain embodiments, the at least one co-stimulatory signaling region comprises an intracellular domain of OX40 or a fragment thereof.

In certain embodiments, the costimulatory molecule is an OX40 polypeptide (e.g., the intracellular domain of OX40 or a fragment thereof). The OX40 polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: P43489 or NP_003318 (SEQ ID NO: 18) or a fragment thereof, and/or may optionally comprise up to one or up to two or up to three conservative amino acid substitutions. SEQ ID NO: 18 is provided below:

[SEQ ID NO: 18]

| | |
|---|---|
| 1 | MCVGARRLGR GPCAALLLLG LGLSTVTGLH CVGDTYPSND |
| | RCCHECRPGN GMVSRCSRSQ |
| 61 | NTVCRPCGPG FYNDVVSSKP CKPCTWCNLR SGSERKQLCT |
| | ATQDTVCRCR AGTQPLDSYK |
| 121 | PGVDCAPCPP GHFSPGDNQA CKPWTNCTLA GKHTLQPASN |
| | SSDAICEDRD PPATQPQETQ |
| 181 | GPPARPITVQ PTEAWPRTSQ GPSTRPVEVP GGRAVAAILG |
| | LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

In certain embodiments, the at least one co-stimulatory signaling region comprises an ICOS polypeptide. In certain embodiments, the at least one co-stimulatory signaling region comprises an intracellular domain of ICOS or a fragment thereof.

In certain embodiments, the costimulatory molecule is an ICOS polypeptide. The ICOS polypeptide can comprise or have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_036224 (SEQ ID NO: 19) or a fragment thereof, and/or may optionally comprise up to one or up to two or up to three conservative amino acid substitutions. SEQ ID NO: 19 is provided below:

[SEQ ID NO: 19]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLTKTKG SGNTVSIKSL KFCHSQLSNN SVSFFLYNLD |
| | HSHANYYFCN LSIFDPPPFK |
| 121 | VTLTGGYLHI YESQLCCQLK FWLPIGCAAF VVVCILGCIL |
| | ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

In certain embodiments, the at least one co-stimulatory signaling region comprises two costimulatory molecules or fragments thereof. In certain embodiments, the at least one co-stimulatory signaling region comprises a CD28 polypeptide (e.g., an intracellular domain of CD28 or a fragment thereof) and a 4-1BB polypeptide (e.g., an intracellular domain of 4-1BB or a fragment thereof).

In certain non-limiting embodiments, the intracellular domain of the membrane-bound polypeptide alone does not provide an activation signal to a cell. In certain embodiments, the intracellular domain of the membrane-bound polypeptide does not comprise a costimulatory molecule. In certain embodiments, the intracellular domain of the membrane-bound polypeptide does not comprise a CD3zeta polypeptide.

In certain embodiments, the intracellular domain of the membrane-bound polypeptide further comprises a suicide gene. Suitable suicide genes include, but are not limited to, Herpes simplex virus thymidine kinase (hsv-tk), and inducible Caspase 9 Suicide gene (iCasp-9). In certain embodiments, the intracellular domain of the membrane-bound polypeptide further comprises a truncated human epidermal growth factor receptor (EGFRt) polypeptide. A truncated EGFRt polypeptide can enable T cell elimination by administering anti-EGFR monoclonal antibody (e.g., cetuximab).

In certain embodiments, the membrane-bound polypeptide comprises a synNotch module. SynNotch modules are disclosed in U.S. Pat. No. 9,670,281 and Morsut et al, Cell, 164, 780-791, 2016, each of which is incorporated by reference in its entirety.

2.2. Soluble Polypeptide

The presently disclosed systems and methods for isolating a cell comprising at least two expression vectors include a membrane-bound polypeptide encoded by a first expression vector, and a soluble polypeptide encoded by a second expression vector. In certain embodiments, the membrane-bound polypeptide is a membrane-bound polypeptide disclosed herein, e.g., in Section 2.1.

In certain embodiments, the soluble polypeptide comprises a dimerization domain that is capable of dimerizing with a dimerization domain comprised in a membrane-bound polypeptide disclosed herein. In certain embodiments, the dimerization domain comprises a leucin zipper domain. The dimerization domain can be any of the dimerization domains disclosed in Section 2.1.1.

In certain embodiments, the soluble polypeptide comprises a dimerization domain and an antigen binding domain that is capable of binding to an antigen.

In certain embodiments, the soluble polypeptide comprises a dimerization domain and a cytokine or a chemokine. In certain embodiments, the soluble polypeptide further comprises a tag.

In certain embodiments, the leucine zipper domain of the membrane-bound polypeptide and the leucine zipper domain of the soluble polypeptide are a pair of orthogonal zippers, i.e., they are the specific partners for each other to form heterodimers.

2.2.1. Cytokine/Chemokine

In certain embodiments, the soluble polypeptide further comprises a cytokine or a chemokine. In certain embodiments, the cytokine/chemokine is capable of enhancing an immune response of an immunoresponsive cell and/or causing cell death of a malignant or infected cell. In certain embodiments, the cytokine/chemokine is an anti-tumor cytokine/chemokine. In certain embodiments, the cytokine or the chemokine comprises or has an amino acid sequence that is at least about 80%, at least about 85%, at least about 90%, at least about 95%, at least about 96%, at least about 97%, at least about 98%, at least about 99% or at least about 100% homologous or identical to a natural cytokine/chemokine, or a fragment thereof, and/or may optionally comprise up to one or up to two or up to three conservative amino acid substitutions. Non-limiting example of cytokines include IL-1, IL-2, IL-3, IL-7, IL-10, IL-12, IL-15, IL-17, IL-18, IL-21, IL-22, IL-36, granulocyte macrophage colony-stimulating factor (GM-CSF), IFN-γ, CXCL1, IL-23, and CXCL10. Non-limiting examples of chemokines include CCL1, CCL8, CCL16, CCL17, CCL18, CCL22, or combinations thereof.

In certain embodiments, the chemokine is CCL1. In certain embodiments, the CCL1 is a mouse CCL1. In certain embodiments, the CCL1 comprises the amino acid sequence set forth in SEQ ID NO: 127. In certain embodiments, the CCL1 is a human CCL1. In certain embodiments, the CCL1 comprises the amino acid sequence set forth in SEQ ID NO: 128. SEQ ID Nos: 127 and 128 are provided below.

[SEQ ID NO: 127]
KSMLTVSNSCCLNTLKKELPLKFIQCYRKMGSSCPDPPAVVFRLNKGRES
CASTNKTWVQNHLKKVNPC
[SEQ ID NO: 128]
KSMQVPFSRCCFSFAEQEIPLRAILCYRNTSSICSNEGLIFKLKRGKEAC
ALDTVGWVQRHRKMLRHCPSKRK

In certain embodiments, the chemokine is CCL17. In certain embodiments, the CCL17 is a mouse CCL17. In certain embodiments, the CCL17 comprises the amino acid sequence set forth in SEQ ID NO: 129. In certain embodiments, the CCL17 is a human CCL17. In certain embodiments, the CCL17 comprises the amino acid sequence set forth in SEQ ID NO: 130. SEQ ID Nos: 129 and 130 are provided below.

[SEQ ID NO: 129]
ARATNVGRECCLDYFKGAIPIRKLVSWYKTSVECSRDAIVFLTVQGKLIC
ADPKDKHVKKAIRLVKNPRP
[SEQ ID NO: 130]
ARGTNVGRECCLEYFKGAIPLRKLKTWYQTSEDCSRDAIVFVTVQGRAIC
SDPNNKRVKNAVKYLQSLERS

In certain embodiments, the chemokine is CCL18. In certain embodiments, the CCL18 is a human CCL18. In certain embodiments, the CCL18 comprises the amino acid sequence set forth in SEQ ID NO: 131. SEQ ID No: 131 is provided below.

[SEQ ID NO: 131]
AQVGTNKELCCLVYTSWQIPQKFIVDYSETSPQCPKPGVILLTKRGRQIC
ADPNKKWVQKYISDLKLNA

In certain embodiments, the chemokine is CCL22. In certain embodiments, the CCL22 is a mouse CCL22. In certain embodiments, the CCL22 comprises the amino acid sequence set forth in SEQ ID NO: 132. In certain embodiments, the CCL22 is a human CCL22. In certain embodiments, the CCL22 comprises the amino acid sequence set forth in SEQ ID NO: 133. SEQ ID Nos: 132 and 133 are provided below.

[SEQ ID NO: 132]
GPYGANVEDSICCQDYIRHPLPSRLVKEFFWTSKSCRKPGVVLITVKNRD
ICADPRQVWVKKLLHKLS
[SEQ ID NO: 133]
GPYGANMEDSVCCRDYVRYRLPLRVVKHFYWTSDSCPRPGVVLLTFRDKE
ICADPRVPWVKMILNKLSQ

2.2.2. Antigen Binding Domain

In certain embodiments, the antigen binding domain of the soluble polypeptide comprises a single-chain variable fragment (scFv), a soluble ligand, a cytokine, or a non-scFv-based antigen recognition motif, or a combination thereof.

In certain non-limiting embodiments, the antigen binding domain of the soluble polypeptide (embodied, for example, an scFv or an analog thereof) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹M or less. In certain non-limiting embodiments, the K_{d} is about 3×10′M or less. In certain non-limiting embodiments, the K_{d} is from about 1×10⁻⁹ M to about 3×10⁻⁷ M. In certain non-limiting embodiments, the K_{d} is from about 1.5×10⁻⁹ M to about 3×10⁻⁷ M.

Binding of the antigen binding domain (for example, in an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In certain embodiments, the antigen binding domain of the soluble polypeptide specifically binds to an antigen. In certain embodiments, the antigen-binding domain is an scFv. In certain embodiments, the scFv is a human scFv. In certain embodiments, the scFv is a humanized scFv. In certain embodiments, the scFv is a murine scFv. In certain embodiments, the antigen-binding domain is a Fab, which is optionally crosslinked. In certain embodiments, the antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

2.2.3. Antigens

In certain embodiments, the antigen binding domain of the soluble polypeptide binds to a tumor antigen. Any tumor antigen can be used in the tumor-related embodiments described herein. The antigen can be expressed as a peptide or as an intact protein or fragment thereof. The intact protein or a fragment thereof can be native or mutagenized. Non-limiting examples of tumor antigens include CD2, CD3, CD4, CD5, CD7, CD8, CD19, CD20, CD22, VpPreB, CD30, CD33, CD38, CD40, CD44v6, CD70, CD79a, CD70b, CLL-1/CLEC12A, CD123, IL-3R complex, TIM-3, BCMA, CD244, E-cadherin, B7-H3, B7-H4, carbonic anhydrase IX (CAlX), carcinoembryonic antigen (CEA), CD10, CD34, CD38, CD41, CD44, CD49f, CD56, CD74, CD133, CD138, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), NKCS1, EGF1R, EGFR-VIII, CD99, ADGRE2, CCR1, LILRB2, PRAME and ERBB.

In certain embodiments, the antigen binding domain of the soluble polypeptide binds to a human CD19 polypeptide. In certain embodiments, the antigen binding domain of the soluble polypeptide binds to the extracellular domain of a human CD19 protein.

In certain embodiments, the antigen binding domain of the soluble polypeptide binds to an immune checkpoint molecule. Non-limiting example of immune checkpoint molecules include PD-L1, CD200, B7-H3, B7-H4, HVEM, Galectin9, PD-1, CTLA-4, CD200R, TIM-3, Lag-3 and TIGIT.

In certain embodiments, the antigen binding domain of the soluble polypeptide binds to an activating receptor, wherein the binding of the antigen binding domain to the activating receptor is capable of activating an antigen presenting cell (APC). Non-limiting example of immune checkpoint molecules include CD40, Toll Like Receptors (TLRs), FLT3, RANK, and GM-CSF receptor.

In certain embodiments, the antigen binding domain of the soluble polypeptide binds to a biomarker of a hematopoietic lineage cell. Non-limiting example of immune checkpoint molecules include CD3, CD16, CD33, c-Kit, CD161, CD19, CD20, vPreB (preB cell receptor), luteinizing hormone receptor (LHCGR), CD123, IL-3R complex, CLEC12A/CLL-1.

In certain embodiments, the antigen binding domain of the soluble polypeptide binds to a pathogen antigen, e.g., for use in treating and/or preventing a pathogen infection or other infectious disease, for example, in an immunocompromised subject. Non-limiting examples of pathogens include a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria and/or fungi include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Aspergillus species and Actinomyces israelli.

2.2.4. Tags

In certain embodiments, the soluble polypeptide comprises a tag. In certain embodiments, the tag comprises an epitope tag, which comprises an epitope recognized by a first antibody. In certain embodiments, the epitope tag is selected from the group consisting of a Myc-tag, a HA-tag, a Flag-tag, a V5-tag, a T7 tag, a CD34 tag, and combinations thereof. In certain embodiments, the tag comprises an affinity tag that binds to a substrate. In certain embodiments, the affinity tag is selected from the group consisting of a His-tag, a Strep-tag, an E-tag, a streptavidin binding protein tag (SBP-tag), and combinations thereof.

2.2.5. Mimotope

In certain embodiments, the soluble polypeptide comprises further comprises a mimotope recognized by a second antibody. Binding of the second antibody to the mimotope can mediate depletion of a cell comprising the soluble polypeptide.

In certain embodiments, the soluble polypeptide comprises an epitope tag recognized by a first antibody and a mimotope recognized by a second antibody. In certain embodiments, the epitope tag is a CD34 epitope tag, the first antibody is an anti-CD34 antibody, the mimotope is a CD20 mimotope, and the second antibody is an anti-CD20 antibody. In certain embodiments, the anti-CD34 antibody is QBEND10. In certain embodiments, the anti-CD20 antibody is Rituximab. In certain embodiments, the CD20 mimotope is a circular CD20 mimotope.

In certain embodiments, the CD20 mimotope comprises or has the amino acid sequence set forth in SEQ ID NO: 116, which is provided below.

[SEQ ID NO: 116]
CPYSNPSLC

In certain embodiments, the CD34 epitope tag comprises or has the amino acid sequence set forth in SEQ ID NO: 117, which is provided below.

[SEQ ID NO: 117]
ELPTQGTFSNVSTNVS

In certain embodiments, the soluble polypeptide comprises two CD34 epitope tags, e.g., each CD34 epitope tag comprises or has the amino acid sequence set forth in SEQ ID NO: 117. In certain embodiments, the two CD34 epitope tags are connected by a linker. In certain embodiments, the linker comprises or has the amino acid sequence set forth in SEQ ID NO: 118, which is provided below.

[SEQ ID NO: 118]
GGGGSGGGS

In certain embodiments, the soluble polypeptide comprises the amino acid sequence set forth in SEQ ID NO: 119, which is provided below. SEQ ID NO: 119 has two CD34 epitope tags, which are linked by the linker having the amino acid sequence set forth in SEQ ID NO: 118, and SEQ ID NO: 119 is referred to “Q2”.

[SEQ ID NO: 119]
ELPTQGTFSNVSTNVSGGGGSGGGSELPTQGTFSNVSTNVS

In certain embodiments, the soluble polypeptide comprises two CD20 mimotopes, e.g., each CD20 mimotope comprises or has the amino acid sequence set forth in SEQ ID NO: 116. In certain embodiments, the two CD20 mimotopes are connected by a linker. In certain embodiments, the linker comprises or has the amino acid sequence set forth in SEQ ID NO: 120, which is provided below.

[SEQ ID NO: 120]
SGGGGSSGGGGSD

In certain embodiments, the soluble polypeptide comprises the amino acid sequence set forth in SEQ ID NO: 121, which is provided below. SEQ ID NO: 121 has two CD20 mimotopes, which are linked by the linker having the amino acid sequence set forth in SEQ ID NO: 120, and SEQ ID NO:121 is referred to “R2”.

[SEQ ID NO: 121]
CPYSNPSLCSGGGGSSGGGGSDCPYSNPSLC

In certain embodiments, the soluble polypeptide comprises two CD20 mimotopes and one CD34 epitope tag, e.g., each CD20 mimotope comprises or has the amino acid sequence set forth in SEQ ID NO: 116, and the CD34 epitope tag comprises or has the amino acid sequence set forth in SEQ ID NO: 117. In certain embodiments, the CD34 epitope tag is linked to each CD20 mimotope with a linker. In certain embodiments, the linker is a human CD8 polypeptide, e.g., one comprising or having the amino acid sequence set forth in SEQ ID NO: 122, which is provided below.

[SEQ ID NO: 122]
PAKPTTT

In certain embodiments, the linker comprises or has the amino acid sequence set forth in SEQ ID NO: 123, which is provided below.

[SEQ ID NO: 123]
SGGGGS

In certain embodiments, the soluble polypeptide comprises the amino acid sequence set forth in SEQ ID NO: 124, which is provided below. SEQ ID NO: 124 has two CD20 mimotopes and one CD34 epitope tag, wherein, the CD34 epitope tag is linked to one CD20 mimotope with a linker having the amino acid sequence set forth in SEQ ID NO: 124, and linked with another CD20 mimotope with a linker having the amino acid sequence set forth in SEQ ID NO: 124, and SEQ ID NO: 124 is referred to “RQR”.

[SEQ ID NO: 124]
CPYSNPSLCSGGGGSELPTQGTFSNVSTNVSPAKPTTTACPYSNPSLC

The presently disclosed subject matter also provides nucleic acids comprising nucleotide sequences encoding presently disclosed polypeptides (e.g., membrane-bound polypeptides or soluble polypeptides). In certain embodiments, the nucleic acids further comprise a promoter, for expressing nucleic acid sequences in human cells. Promoters for use in expressing the polypeptide can be a constitutive promoter (e.g., ubiquitin C (UbiC) promoter, MSCV, SFFV, EF1alpha, RSV, PGK, and MMLV LTR), or an inducible promoter (e.g., a NFAT transcriptional response element (TRE) promoter, a CD69 promoter, a CD25 promoter, an IL-2 promoter, an IL-6 response element, an sis inducible element (SIE), an Interferon gamma response element, a GAS/IRES element, a NFkB response element, a Gal response element, and a tetracycline response element).

Further provided herein are xpression vectors comprising nucleic acid molecules encoding membrane-bound polypeptides as disclosed herein, or soluble polypeptides as disclosed herein. The expression vector can be a viral vector or a transposon-based vector. In certain embodiments, the viral vector is a retroviral vector. In certain embodiments, the retroviral vector is a lentiviral vector. The presently disclosed subject matter also provides host cells comprising the presently disclosed nucleic acid molecules. In certain embodiments, the host cell is a T cell.

### 3. Systems

The presently disclosed subject matter provides systems for isolating a cell and/or immunotherapy, e.g., for isolating a cell comprising at least two expression vectors. In certain embodiments, the system comprises a presently disclosed membrane-bound polypeptide encoded by a first expression vector, and a presently disclosed soluble polypeptide encoded by a second expression vector.

3.1. Cell Sorting System Comprising a Membrane-Bound Polypeptide Comprising a Self-Blocking Feature

The presently disclosed subject matter provides a system for isolating a cell comprising at least two expression vectors. In certain embodiments, the system comprise: a) a presently disclosed membrane-bound polypeptide encoded by a first expression vector, and b) a presently disclosed soluble polypeptide encoded by a second expression vector. In certain embodiments, the soluble polypeptide comprises a tag and a third dimerization domain that is capable of dimerizing with the first dimerization domain. In certain embodiments, the third dimerization domain forms a dimer with the first dimerization domain prior to the dimerization between the first dimerization domain and the second dimerization domain. In certain embodiments, the third dimerization domain forms a dimer with the first dimerization domain in the endoplasmic reticulum. In certain embodiments, the soluble polypeptide and the membrane-bound polypeptide are capable of forming a dimer when expressed from the same cell. In certain embodiments, the soluble polypeptide and the membrane-bound polypeptide are capable of forming a dimer in the endoplasmic reticulum, when expressed from the same cell. In certain embodiments, the soluble polypeptide and the membrane-bound polypeptide are not capable of forming a dimer when expressed from different cells due to the dimerization between the first dimerization domain and the second dimerization domain. In certain embodiments, the third dimerization domain comprises the amino acid sequence set forth in SEQ ID NO: 1, SEQ ID NO: 2, or SEQ ID NO: 106.

3.2. Cell Sorting System Comprising a Membrane-Bound Polypeptide Comprising a Blocking Spacer

The presently disclosed subject matter provides a system for isolating a cell comprising at least two expression vectors. In certain embodiments, the system comprise: a) a membrane-bound polypeptide encoded by a first expression vector, wherein the membrane-bound polypeptide comprises a transmembrane domain and an extracellular domain, wherein that extracellular domain comprises a first dimerization domain and a blocking spacer, and b) a soluble polypeptide encoded by a second expression vector, wherein the soluble polypeptide comprises a tag and a second dimerization domain. In certain embodiments, each of the first and second dimerization domains comprises a leucine zipper domain, and wherein the blocking spacer prevents dimerization of the membrane-bound polypeptide with the soluble polypeptide when the membrane-bound polypeptide and the soluble polypeptide are not expressed from the same cell. In certain embodiments, the first dimerization domain comprises the amino acid sequence set forth in SEQ ID NO: 1, SEQ ID NO: 2, or SEQ ID NO: 106. In certain embodiments, the second dimerization domain comprises the amino acid sequence set forth in SEQ ID NO: 1, SEQ ID NO: 2, or SEQ ID NO: 106. In certain embodiments, the blocking spacer is no more than about 25 amino acid residues. In certain embodiments, the blocking spacer is between about 5 amino acid residues and about 25 amino acid residues. In certain embodiments, the blocking spacer is a truncated CD28 spacer or an IgG1 hinge.

3.3. Common Features of the Systems Disclosed Herein

Any features of the membrane-bound polypeptides or soluble polypeptides disclosed herein (e.g., as disclosed in Section 2) can be applied to the systems disclosed herein.

In certain embodiments, the tag comprises an epitope tag recognized by a first antibody. In certain embodiments, the epitope tag is selected from the group consisting of a Myc-tag, a HA-tag, a Flag-tag, a V5-tag, a T7 tag, a CD34-tag, and combinations thereof. In certain embodiments, the tag comprises an affinity tag that binds to a substrate. In certain embodiments, the affinity tag is selected from the group consisting of a His-tag, a Strep-tag, an E-tag, a streptavidin binding protein tag (SBP-tag), and, combinations thereof.

In certain embodiments, the soluble polypeptide further comprises an antigen binding domain. In certain embodiments, the antigen binding domain comprises a single-chain variable fragment (scFv), a soluble ligand, a cytokine, a chemokine, a non-scFv-based antigen recognition motif, or a combination thereof. In certain embodiments, the soluble polypeptide further comprises a cytokine or a chemokine.

In certain embodiments, the membrane-bound polypeptide is expressed from a first vector. In certain embodiments, the soluble polypeptide is expressed from a second vector. The first vector can be the same as the second vector, or be different from the second vector. In certain embodiments, the first vector is the same as the second vector, for example, the vector backbone for the first and the second vectors can be the same, while the polypeptide or protein encoded/expressed by the first and second vectors can be different.

3.4. Exemplary Membrane-Bound Polypeptides, Soluble Polypeptides, and Systems

In certain embodiments, the membrane-bound polypeptide comprises a V5 tag, an EE12RR345L leucine zipper, a CD28EC-9C hinge, a CD2 transmembrane (TM) domain and truncated cytoplasmic domain, an E2A peptide, and a Thy1.1 peptide. (V5 tag staining identifies the membrane-bound polypeptide surface expression). In certain embodiments, the membrane-bound polypeptide comprises the amino acid sequence set forth in SEQ ID NO: 24.

[SEQ ID NO: 24]
METDTLLLWVLLLWVPGSTGGSGGKPIPNPLLGLDSTGGGGSGGGSLEIE
AAFLERENTALETRVAELRQRVQRLRNRVSQYRTRYGPLGGGKAAACHTQ
SSPKLGSLEYVTVGVGAGGLLLVLLVALFIFCICKRRKGTGQCTNYALLK
LAGDVESNPGPGSMNPAISVALLLSVLQVSRGQKVTSLTACLVNQNLRLD
CRHENNTKDNSIQHEFSLTREKRKHVLSGTLGIPEHTYRSRVTLSNQPYI
KVLTLANFTTKDEGDYFCELRVSGANPMSSNKSISVYRDKLVKCGGISLL
VQNTSWMLLLLLSLSLLQALDFISL

In certain embodiments, the membrane-bound polypeptide comprises a V5 tag, an EE12RR345L leucine zipper, a CD28EC-9C hinge, a CD28 TM domain, a CD3z delta domain, an E2A peptide, and a Thy1.1 peptide. (V5 tag staining identifies the membrane-bound polypeptide surface expression). In certain embodiments, the membrane-bound polypeptide comprises the amino acid sequence set forth in SEQ ID NO: 25.

[SEQ ID NO: 25]
METDTLLLWVLLLWVPGSTGGSGGKPIPNPLLGLDSTGGGGSGGGSLEIE
AAFLERENTALETRVAELRQRVQRLRNRVSQYRTRYGPLGGGKAAACHTQ
SSPKLGSLFWALVVVAGVLECYGLLVTVALCVIWTRAKESRSAETAANLQ
DTFDALHMQTLAPRRAKRGTGQCTNYALLKLAGDVESNPGPGSMNPAISV
ALLLSVLQVSRGQKVTSLTACLVNQNLRLDCRHENNTKDNSIQHEFSLTR
EKRKHVLSGTLGIPEHTYRSRVTLSNQPYIKVLTLANFTTKDEGDYFCEL
RVSGANPMSSNKSISVYRDKLVKCGGISLLVQNTSWMLLLLLSLSLLQAL
DFISL

In certain embodiments, the membrane-bound polypeptide comprises an EE12RR345L leucine zipper, a myc tag, a CD28EC-9C hinge, a CD2 TM domain and truncated cytoplasmic domain, an E2A peptide, and a Thy1.1 peptide. (No myc staining). In certain embodiments, the membrane-bound polypeptide comprises the amino acid sequence set forth in SEQ ID NO: 26.

[SEQ ID NO: 26]
METDTLLLWVLLLWVPGSTGLEIEAAFLERENTALETRVAELRQRVQRLR
NRVSQYRTRYGPLGGGKGSGEQKLISEEDLAAACHTQSSPKLGSLFYVTV
GVGAGGLLLVLLVALFIFCICKRRKGTGQCTNYALLKLAGDVESNPGPGS
MNPAISVALLLSVLQVSRGQKVTSLTACLVNQNLRLDCRHENNTKDNSIQ
HEFSLTREKRKHVLSGTLGIPEHTYRSRVTLSNQPYIKVLTLANFTTKDE
GDYFCELRVSGANPMSSNKSISVYRDKLVKCGGISLLVQNTSWMLLLLLS
LSLLQALDFISL

In certain embodiments, the membrane-bound polypeptide comprises an EE12RR345L leucine zipper, a myc tag, a CD28EC-9C hinge, a CD28 TM domain, a CD3z delta domain, an E2A peptide, and a Thy1.1 peptide. (No myc staining). In certain embodiments, the membrane-bound polypeptide comprises the amino acid sequence set forth in SEQ ID NO: 27.

[SEQ ID NO: 27]
METDTLLLWVLLLWVPGSTGLEIEAAFLERENTALETRVAELRQRVQRLR
NRVSQYRTRYGPLGGGKGSGEQKLISEEDLAAACHTQSSPKLGSLFWALV
VVAGVLECYGLLVTVALCVIWTRAKFSRSAETAANLQDTFDALHMQTLAP
RRAKRGTGQCTNYALLKLAGDVESNPGPGSMNPAISVALLLSVLQVSRGQ
KVTSLTACLVNQNLRLDCRHENNTKDNSIQHEFSLTREKRKHVLSGTLGI
PEHTYRSRVTLSNQPYIKVLTLANFTTKDEGDYFCELRVSGANPMSSNKS
ISVYRDKLVKCGGISLLVQNTSWMLLLLLSLSLLQALDFISL

In certain embodiments, the membrane-bound polypeptide comprises a V5 tag, an EE12RR345L leucine zipper, an IgG1 hinge, a CD2 TM domain and truncated cytoplasmic domain, an E2A peptide, and a Thy1.1 peptide. (V5 tag staining identifies the membrane-bound polypeptide surface expression). In certain embodiments, the membrane-bound polypeptide comprises the amino acid sequence set forth in SEQ ID NO: 28.

[SEQ ID NO: 28]
METDTLLLWVLLLWVPGSTGGSGGKPIPNPLLGLDSTGGGGSGGGSLET
EAAFLERENTALETRVAELRQRVQRLRNRVSQYRTRYGPLGGGKAAAVP
RDCGCKPCICTGSLEYVTVGVGAGGLLLVLLVALFIFCICKRRKGTGQC
TNYALLKLAGDVESNPGPGSMNPAISVALLLSVLQVSRGQKVTSLTACL
VNQNLRLDCRHENNTKDNSIQHEFSLTREKRKHVLSGTLGIPEHTYRSR
VTLSNQPYIKVLTLANFTTKDEGDYFCELRVSGANPMSSNKSISVYRDK
LVKCGGISLLVQNTSWMLLLLLSLSLLQALDFISL

In certain embodiments, the membrane-bound polypeptide comprises a V5 tag, an EE12RR345L leucine zipper, an IgG1 hinge, a CD28 TM domain, a CD3ζ delta domain, an E2A peptide, and a Thy1.1 peptide. (V5 tag staining identifies the membrane-bound polypeptide surface expression). In certain embodiments, the membrane-bound polypeptide comprises the amino acid sequence set forth in SEQ ID NO: 29.

[SEQ ID NO: 29]
METDTLLLWVLLLWVPGSTGGSGGKPIPNPLLGLDSTGGGGSGGGSLEI
EAAFLERENTALETRVAELRQRVQRLRNRVSQYRTRYGPLGGGKAAAVP
RDCGCKPCICTGSLFWALVVVAGVLFCYGLLVTVALCVIWTRAKFSRSA
ETAANLQDTFDALHMQTLAPRRAKRGTGQCTNYALLKLAGDVESNPGPG
SMNPAISVALLLSVLQVSRGQKVTSLTACLVNQNLRLDCRHENNTKDNS
IQHEFSLTREKRKHVLSGTLGIPEHTYRSRVTLSNQPYIKVLTLANFTT
KDEGDYFCELRVSGANPMSSNKSISVYRDKLVKCGGISLLVQNTSWMLL
LLLSLSLLQALDFISL

In certain embodiments, the system comprises a soluble polypeptide (e.g., an RR12EE345L leucine zipper), a linker, a membrane-bound polypeptide (e.g., an EE12RR345L leucine zipper), an EGFRt polypeptide, a P2A peptide, and a blue fluorescent protein (BFP). In certain embodiments, the system comprises the amino acid sequence set forth in SEQ ID NO: 30.

[SEQ ID NO: 30]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTRKVCNGIGIGE
FKDSLSINATNIKHFKNCTSISGDLHILPVAFRGDSFTHTPPLDPQELD
ILKTVKEITGFLLIQAWPENRTDLHAFENLEIIRGRTKQHGQFSLAVVS
LNITSLGLRSLKEISDGDVIISGNKNLCYANTINWKKLEGTSGQKTKII
SNRGENSCKATGQVCHALCSPEGCWGPEPRDCVSCRNVSRGRECVDKCN
LLEGEPREFVENSECIQCHPECLPQAMNITCTGRGPDNCIQCAHYIDGP
HCVKTCPAGVMGENNTLVWKYADAGHVCHLCHPNCTYGCTGPGLEGCPT
NGPKIPSIATGMVGALLLLLVVALGIGLFMRDGSGRAKRATNFSLLKQA
GDVEENPGPGSLMSELIKENMHMKLYMEGTVDNHHFKCTSEGEGKPYEG
TQTMRIKVVEGGPLPFAFDILATSFLYGSKTFINHTQGIPDFFKQSFPE
GFTWERVTTYEDGGVLTATQDTSLQDGCLIYNVKIRGVNFTSNGPVMQK
KTLGWEAFTETLYPADGGLEGRNDMALKLVGGSHLIANIKTTYRSKKPA
KNLKMPGVYYVDYRLERIKEANNETYVEQHEVAVARYCDLPSKLGHKLN

In certain embodiments, the system comprises a soluble polypeptide (e.g., an RR12EE345L leucine zipper), a linker, a membrane-bound polypeptide (e.g., an EE12RR345L leucine zipper), a Thy1.1 polypeptide, a P2A peptide, and a blue fluorescent protein (BFP). In certain embodiments, the system comprises the amino acid sequence set forth in SEQ ID NO: 31.

[SEQ ID NO: 31]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANFTTKDEGDYFCELRVSGANPMSSNKSISVYRDKL
VKCGGISLLVQNTSWMLLLLLSLSLLQALDFISLGSGATNFSLLKQAGD
VEENPGPGSLMSELIKENMHMKLYMEGTVDNHHFKCTSEGEGKPYEGTQ
TMRIKVVEGGPLPFAFDILATSFLYGSKTFINHTQGIPDFFKQSFPEGF
TWERVTTYEDGGVLTATQDTSLQDGCLIYNVKIRGVNFTSNGPVMQKKT
LGWEAFTETLYPADGGLEGRNDMALKLVGGSHLTANIKTTYRSKKPAKN
LKMPGVYYVDYRLERIKEANNETYVEQHEVAVARYCDLPSKLGHKLN

In certain embodiments, the membrane-bound polypeptide comprises an EE12RR345L leucine zipper, a myc tag, an MHC-I hinge/TM domain, a P2A peptide, and a blue fluorescent protein (BFP). (No myc staining). In certain embodiments, the membrane-bound polypeptide comprises the amino acid sequence set forth in SEQ ID NO: 32.

[SEQ ID NO: 32]
METDTLLLWVLLLWVPGSTGLEIEAAFLERENTALETRVAELRQRVQRL
RNRVSQYRTRYGPLGGGKEFTEQKLISEEDLVIIAVLVVLGAAIVTGAV
VAFVMKGSGATNFSLLKQAGDVEENPGPGSLMSELIKENMHMKLYMEGT
VDNHHFKCTSEGEGKPYEGTQTMRIKVVEGGPLPFAFDILATSFLYGSK
TFINHTQGIPDFFKQSFPEGFTWERVTTYEDGGVLTATQDTSLQDGCLI
YNVKIRGVNFTSNGPVMQKKTLGWEAFTETLYPADGGLEGRNDMALKLV
GGSHLTANIKTTYRSKKPAKNLKMPGVYYVDYRLERIKEANNETYVEQH
EVAVARYCDLPSKLGHKLN

In certain embodiments, the soluble polypeptide comprises a FLAG tagged RR12EE345L leucine zipper, a P2A peptide, an iCaspase9 polypeptide, and a F2A peptide. In certain embodiments, the soluble polypeptide construct comprises the amino acid sequence set forth in SEQ ID NO: 33.

[SEQ ID NO: 33]
METDTLLLWVLLLWVPGSTGDYKDDDDKLEIRAAFLRQRNTALRTEVAE
LEQEVQRLENEVSQYETRYGPLGGGKGSGRAKRATNFSLLKQAGDVEEN
PGPEFTRAKRMGVQVETISPGDGRTFPKRGQTCVVHYTGMLEDGKKVDS
SRDRNKPFKFMLGKQEVIRGWEEGVAQMSVGQRAKLTISPDYAYGATGH
PGIIPPHATLVEDVELLKLESGGGSGVDGEGDVGALESLRGNADLAYIL
SMEPCGHCLIINNVNECRESGLRTRTGSNIDCEKLRRRESSLHFMVEVK
GDLTAKKMVLALLELARQDHGALDCCVVVILSHGCQASHLQFPGAVYGT
DGCPVSVEKIVNIFNGTSCPSLGGKPKLFFIQACGGEQKDHGFEVASTS
PEDESPGSNPEPDATPFQEGLRTFDQLDAISSLPTPSDIFVSYSTFPGF
VSWRDPKSGSWYVETLDDIFEQWAHSEDLQSLLLRVANAVSVKGIYKQM
PGCFNFLRKKLFFKTSVDGTGRAKRVKQTLNFDLLKLAGDVESNPGPGS
L

In certain embodiments, the soluble polypeptide comprises a FLAG tagged RR12EE345L leucine zipper, a P2A peptide, an iCaspase9 polypeptide, a F2A peptide, a CD19 scFv, a myc tag, a CD8EC hinge/TM domain, a CD28 costimulatory domain, and a CD3z polypeptide. In certain embodiments, the soluble polypeptide comprises the amino acid sequences set forth in SEQ ID NOs: 34 and 35.

[SEQ ID NO: 34]
METDTLLLWVLLLWVPGSTGDYKDDDDKLEIRAAFLRQRNTALRTEVAE
LEQEVQRLENEVSQYETRYGPLGGGKGSGRAKRATNFSLLKQAGDVEEN
PGPEFTRAKRMGVQVETISPGDGRTFPKRGQTCVVHYTGMLEDGKKVDS
SRDRNKPFKFMLGKQEVIRGWEEGVAQMSVGQRAKLTISPDYAYGATGH
PGIIPPHATLVEDVELLKLESGGGSGVDGEGDVGALESLRGNADLAYIL
SMEPCGHCLIINNVNECRESGLRTRTGSNIDCEKLRRRESSLHEMVEVK
GDLTAKKMVLALLELARQDHGALDCCVVVILSHGCQASHLQFPGAVYGT
DGCPVSVEKIVNIENGTSCPSLGGKPKLFFIQACGGEQKDHGFEVASTS
PEDESPGSNPEPDATPFQEGLRTFDQLDAISSLPTPSDIFVSYSTFPGF
VSWRDPKSGSWYVETLDDIFEQWAHSEDLQSLLLRVANAVSVKGIYKQM
PGCFNFLRKKLEFKTSVDGTGRAKRVKQTLNEDLLKLAGDVESNPGPGS
LT
[SEQ ID NO: 35]
EQKLISEEDLLESSTTTKPVLRTPSPVHPTGTSQPQRPEDCRPRGSVKG
TGLDFACDIYIWAPLAGICVALLLSLIITLICYNSRRNRLLQSDYMNMT
PRRPGLTRKPYQPYAPARDFAAYRPRAKFSRSAETAANLQDPNQLYNEL
NLGRREEYDVLEKKRARDPEMGGKQQRRRNPQEGVYNALQKDKMAEAYS
EIGTKGERRRGKGHDGLYQGLSTATKDTYDALHMQTLAPRGTEARL

In certain embodiments, the soluble polypeptide comprises a FLAG tagged RR12EE345L leucine zipper, a P2A peptide, an iCaspase9 polypeptide, a F2A peptide, an IL-3 polypeptide, a CD8EC hinge/TM domain, a CD28 costimulatory domain, and a CD3ζ polypeptide. In certain embodiments, the soluble polypeptide comprises the amino acid sequence set forth in SEQ ID NO: 36.

[SEQ ID NO: 36]
METDTLLLWVLLLWVPGSTGDYKDDDDKLEIRAAFLRQRNTALRTEVAE
LEQEVQRLENEVSQYETRYGPLGGGKGSGRAKRATNFSLLKQAGDVEEN
PGPEFTRAKRMGVQVETISPGDGRTFPKRGQTCVVHYTGMLEDGKKVDS
SRDRNKPFKFMLGKQEVIRGWEEGVAQMSVGQRAKLTISPDYAYGATGH
PGIIPPHATLVEDVELLKLESGGGSGVDGEGDVGALESLRGNADLAYIL
SMEPCGHCLIINNVNECRESGLRTRTGSNIDCEKLRRRESSLHEMVEVK
GDLTAKKMVLALLELARQDHGALDCCVVVILSHGCQASHLQFPGAVYGT
DGCPVSVEKIVNIENGTSCPSLGGKPKLFFIQACGGEQKDHGFEVASTS
PEDESPGSNPEPDATPFQEGLRTFDQLDAISSLPTPSDIFVSYSTFPGF
VSWRDPKSGSWYVETLDDIFEQWAHSEDLQSLLLRVANAVSVKGIYKQM
PGCFNFLRKKLEFKTSVDGTGRAKRVKQTLNEDLLKLAGDVESNPGPGS
LTMASPLTRFLSLNLLLLGESIILGSGEAASISGRDTHRLTRTLNCSSI
VKEIIGKLPEPELKTDDEGPSLRNKSERRVNLSKFVESQGEVDPEDRYV
IKSNLQKLNCCLPTSANDSALPGVFIRDLDDERKKLREYMVHLNDLETV
LTSRPPQPASGSVSPNRGTVECLESSTTTKPVLRTPSPVHPTGTSQPQR
PEDCRPRGSVKGTGLDFACDIYIWAPLAGICVALLLSLIITLICYNSRR
NRLLQSDYMNMTPRRPGLTRKPYQPYAPARDFAAYRPRAKFSRSAETAA
NLQDPNQLYNELNLGRREEYDVLEKKRARDPEMGGKQQRRRNPQEGVYN
ALQKDKMAEAYSEIGTKGERRRGKGHDGLYQGLSTATKDTYDALHMQTL
APRGTEARL

In certain embodiments, the soluble polypeptide comprises a FLAG tagged RR12EE345L leucine zipper, a P2A peptide, an iCaspase9 polypeptide, a F2A peptide, a CD38 scFv, an interchain linker, an IL-3 polypeptide, a CD8EC hinge/TM domain, a CD28 costimulatory domain, and a CD3z polypeptide. In certain embodiments, the soluble polypeptide comprises the amino acid sequences set forth in SEQ ID NOs: 37 and 38.

[SEQ ID NO: 37]
METDTLLLWVLLLWVPGSTGDYKDDDDKLEIRAAFLRQRNTALRTEVAE
LEQEVQRLENEVSQYETRYGPLGGGKGSGRAKRATNFSLLKQAGDVEEN
PGPEFTRAKRMGVQVETISPGDGRTFPKRGQTCVVHYTGMLEDGKKVDS
SRDRNKPFKFMLGKQEVIRGWEEGVAQMSVGQRAKLTISPDYAYGATGH
PGIIPPHATLVEDVELLKLESGGGSGVDGEGDVGALESLRGNADLAYIL
SMEPCGHCLIINNVNECRESGLRTRTGSNIDCEKLRRRESSLHFMVEVK
GDLTAKKMVLALLELARQDHGALDCCVVVILSHGCQASHLQFPGAVYGT
DGCPVSVEKIVNIFNGTSCPSLGGKPKLFFIQACGGEQKDHGFEVASTS
PEDESPGSNPEPDATPFQEGLRTFDQLDAISSLPTPSDIFVSYSTFPGF
VSWRDPKSGSWYVETLDDIFEQWAHSEDLQSLLLRVANAVSVKGIYKQM
PGCFNFLRKKLEFKTSVDGTGRAKRVKQTLNEDLLKLAGDVESNPGPGS
LT
[SEQ ID NO: 38]
GTGGSTGGGGSGGGGSGGGGSASISGRDTHRLTRTLNCSSIVKEIIGKL
PEPELKTDDEGPSLRNKSERRVNLSKFVESQGEVDPEDRYVIKSNLQKL
NCCLPTSANDSALPGVFIRDLDDERKKLREYMVHLNDLETVLTSRPPQP
ASGSVSPNRGTVECLESSTTTKPVLRTPSPVHPTGTSQPQRPEDCRPRG
SVKGTGLDFACDIYIWAPLAGICVALLLSLIITLICYNSRRNRLLQSDY
MNMTPRRPGLTRKPYQPYAPARDFAAYRPRAKFSRSAETAANLQDPNQL
YNELNLGRREEYDVLEKKRARDPEMGGKQQRRRNPQEGVYNALQKDKMA
EAYSEIGTKGERRRGKGHDGLYQGLSTATKDTYDALHMQTLAPRGTEAR
L

In certain embodiments, the system comprises a soluble polypeptide (e.g., RR12EE345L leucine zipper), a linker, a membrane-bound polypeptide (e.g. an EE12RR345L leucine zipper), a Thy1.1 polypeptide, a P2A peptide, a CD20 scFv, a twin streptag-II, a CD8EC hinge/TM domain, a CD28 costimulatory domain, a CD3ζ polypeptide and an E2A polypeptide. In certain embodiments, the system comprises the amino acid sequences set forth in SEQ ID NOs: 39 and 40.

[SEQ ID NO: 39]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANFTTKDEGDYFCELRVSGANPMSSNKSISVYRDKL
VKCGGISLLVQNTSWMLLLLLSLSLLQALDFISLGSGATNFSLLKQAGD
VEENPGPGSLT
[SEQ ID NO: 40]
SAWSHPQFEKGGGSGGGSGGSAWSHPQFEKGGGGSGGGSEARLSTTTKP
VLRTPSPVHPTGTSQPQRPEDCRPRGSVKGTGLDFACDIYIWAPLAGIC
VALLLSLIITLICYNSRRNRLLQSDYMNMTPRRPGLTRKPYQPYAPARD
FAAYRPRAKFSRSAETAANLQDPNQLYNELNLGRREEYDVLEKKRARDP
EMGGKQQRRRNPQEGVYNALQKDKMAEAYSEIGTKGERRRGKGHDGLYQ
GLSTATKDTYDALHMQTLAPRGSGQCTNYALLKLAGDVESNPGPLE

In certain embodiments, the system comprises a soluble polypeptide (e.g., RR12EE345L leucine zipper), a linker, a membrane-bound polypeptide (e.g. an EE12RR345L leucine zipper), a Thy1.1 polypeptide, a P2A peptide, a CD20 scFv, a twin streptag-II, a CD8EC hinge/TM domain, a CD28 costimulatory domain, a CD3ζ polypeptide, an E2A polypeptide and a pro-IL-18 polypeptide (contains IL-18 pro-peptide sequence). In certain embodiments, the system comprises the amino acid sequences set forth in SEQ ID NOs: 41 and 42.

[SEQ ID NO: 41]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANFTTKDEGDYFCELRVSGANPMSSNKSISVYRDKL
VKCGGISLLVQNTSWMLLLLLSLSLLQALDFISLGSGATNFSLLKQAGD
VEENPGPGSLT
[SEQ ID NO: 42]
SAWSHPQFEKGGGSGGGSGGSAWSHPQFEKGGGGSGGGSEARLSTTTKP
VLRTPSPVHPTGTSQPQRPEDCRPRGSVKGTGLDFACDIYIWAPLAGIC
VALLLSLIITLICYNSRRNRLLQSDYMNMTPRRPGLTRKPYQPYAPARD
FAAYRPRAKESRSAETAANLQDPNQLYNELNLGRREEYDVLEKKRARDP
EMGGKQQRRRNPQEGVYNALQKDKMAEAYSEIGTKGERRRGKGHDGLYQ
GLSTATKDTYDALHMQTLAPRGSGQCTNYALLKLAGDVESNPGPLESMA
AMSEDSCVNFKEMMFIDNTLYFIPEENGDLESDNFGRLHCTTAVIRNIN
DQVLFVDKRQPVFEDMTDIDQSASEPQTRLIIYMYKDSEVRGLAVTLSV
KDSKMSTLSCKNKIISFEEMDPPENTDDIQSDLIFFQKRVPGHNKMEFE
SSLYEGHFLACQKEDDAFKLILKKKDENGDKSVMFTLTNLHQS

In certain embodiments, the system comprises a soluble polypeptide (e.g., RR12EE345L leucine zipper), a linker, a membrane-bound polypeptide (e.g. an EE12RR345L leucine zipper), a Thy1.1 polypeptide, a P2A peptide, a CD20 scFv, a twin streptag-II, a CD8EC hinge/TM domain, a CD28 costimulatory domain, a CD3ζ polypeptide, an E2A polypeptide and a sIL-18 polypeptide (contains mouse IL-2 signal peptide sequence). In certain embodiments, the system comprises the amino acid sequences set forth in SEQ ID NOs: 43 and 44.

[SEQ ID NO: 43]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANETTKDEGDYFCELRVSGANPMSSNKSISVYRDKL
VKCGGISLLVQNTSWMLLLLLSLSLLQALDFISLGSGATNFSLLKQAGD
VEENPGPGSLT
[SEQ ID NO: 44]
SAWSHPQFEKGGGSGGGSGGSAWSHPQFEKGGGGSGGGSEARLSTTTKP
VLRTPSPVHPTGTSQPQRPEDCRPRGSVKGTGLDFACDIYIWAPLAGIC
VALLLSLIITLICYNSRRNRLLQSDYMNMTPRRPGLTRKPYQPYAPARD
FAAYRPRAKESRSAETAANLQDPNQLYNELNLGRREEYDVLEKKRARDP
EMGGKQQRRRNPQEGVYNALQKDKMAEAYSEIGTKGERRRGKGHDGLYQ
GLSTATKDTYDALHMQTLAPRGSGQCTNYALLKLAGDVESNPGPLESMY
SMQLASCVTLTLVLLVNSNFGRLHCTTAVIRNINDQVLFVDKRQPVFED
MTDIDQSASEPQTRLIIYMYKDSEVRGLAVTLSVKDSKMSTLSCKNKII
SFEEMDPPENIDDIQSDLIFFQKRVPGHNKMEFESSLYEGHFLACQKED
DAFKLILKKKDENGDKSVMFTLTNLHQS

In certain embodiments, the system comprises a soluble polypeptide (e.g., RR12EE345L leucine zipper), a linker, a membrane-bound polypeptide (e.g. an EE12RR345L leucine zipper), a Thy1.1 polypeptide, a P2A peptide, a CD20 scFv, an interchain linker, a CD19 scFv, a myc tag, a CD8EC hinge/TM domain, a CD28 costimulatory domain, a CD3ζ polypeptide, and an E2A polypeptide. In certain embodiments, the system comprises the amino acid sequences set forth in SEQ ID NOs: 45, 46 and 47.

[SEQ ID NO: 45]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANFTTKDEGDYFCELRVSGANPMSSNKSISVYRDKL
VKCGGISLLVQNTSWMLLLLLSLSLLQALDFISLGSGATNFSLLKQAGD
VEENPGPGSLT
[SEQ ID NO: 46]
GTGGSTGGGGSGGGGSGGGGS
[SEQ ID NO: 47]
EQKLISEEDLEARLSTTTKPVLRTPSPVHPTGTSQPQRPEDCRPRGSVK
GTGLDFACDIYIWAPLAGICVALLLSLIITLICYNSRRNRLLQSDYMNM
TPRRPGLTRKPYQPYAPARDFAAYRPRAKFSRSAETAANLQDPNQLYNE
LNLGRREEYDVLEKKRARDPEMGGKQQRRRNPQEGVYNALQKDKMAEAY
SEIGTKGERRRGKGHDGLYQGLSTATKDTYDALHMQTLAPRGSGQCTNY
ALLKLAGDVESNPGPLE

In certain embodiments, the system comprises a soluble polypeptide (e.g., RR12EE345L leucine zipper), a linker, a membrane-bound polypeptide (e.g. an EE12RR345L leucine zipper), a Thy1.1 polypeptide, a P2A peptide, a CD20 scFv, an interchain linker, an IL-3 polypeptide, a twin streptag-II, a CD8EC hinge/TM domain, a CD28 costimulatory domain, a CD3ζ polypeptide, and an E2A polypeptide. In certain embodiments, the system comprises the amino acid sequences set forth in SEQ ID NOs: 48 and 49.

[SEQ ID NO: 48]
METDTLLLWVLLLWVPGSTGLEIRAAFLRQRNTALRTEVAELEQEVQRL
ENEVSQYETRYGPLGGGKGGGGSGGGGSGGGGSGGGSLEIEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANFTTKDEGDYFCELRVSGANPMSSNKSISVYRDKL
VKCGGISLLVQNTSWMLLLLLSLSLLQALDFISLGSGATNFSLLKQAGD
VEENPGPGSLT
[SEQ ID NO: 49]
GTGGSTGGGGSGGGGSGGGGSASISGRDTHRLTRTLNCSSIVKETIGKL
PEPELKTDDEGPSLRNKSFRRVNLSKFVESQGEVDPEDRYVIKSNLQKL
NCCLPTSANDSALPGVFIRDLDDFRKKLRFYMVHLNDLETVLTSRPPQP
ASGSVSPNRGTVECSAWSHPQFEKGGGSGGGSGGSAWSHPQFEKGGGGS
GGGGEARLSTTTKPVLRTPSPVHPTGTSQPQRPEDCRPRGSVKGTGLDF
ACDIYIWAPLAGICVALLLSLIITLICYNSRRNRLLQSDYMNMTPRRPG
LTRKPYQPYAPARDFAAYRPRAKFSRSAETAANLQDPNQLYNELNLGRR
EEYDVLEKKRARDPEMGGKQQRRRNPQEGVYNALQKDKMAEAYSEIGTK
GERRRGKGHDGLYQGLSTATKDTYDALHMQTLAPRGSGQCTNYALLKLA
GDVESNPGPLE

In certain embodiments, the system comprises a soluble polypeptide (e.g., RR12EE345L leucine zipper), a linker, a membrane-bound polypeptide (e.g. an EE12RR345L leucine zipper), a Thy1.1 polypeptide, a P2A peptide, an IL-3 polypeptide, an interchain linker, a CD20 scFv, a twin streptag-II, a CD8EC hinge/TM domain, a CD28 costimulatory domain, a CD3ζ polypeptide, and an E2A polypeptide. In certain embodiments, the system comprises the amino acid sequences set forth in SEQ ID NOs: 50 and 51.

[SEQ ID NO: 50]
METDTLLLWVLLLWVPGSTGLEIRAAFLRQRNTALRTEVAELEQEVQRL
ENEVSQYETRYGPLGGGKGGGGSGGGGSGGGGSGGGSLETEAAFLEREN
TALETRVAELRQRVQRLRNRVSQYRTRYGPLGGGKEFTQKVTSLTACLV
NQNLRLDCRHENNTKDNSIQHEFSLTREKRKHVLSGTLGIPEHTYRSRV
TLSNQPYIKVLTLANFTTKDEGDYFCELRVSGANPMSSNKSISVYRDKL
VKCGGISLLVQNTSWMLLLLLSLSLLQALDFISLGSGATNESLLKQAGD
VEENPGPGSLTMASPLTRELSLNLLLLGESTILGSGEAASISGRDTHRL
TRTLNCSSIVKEIIGKLPEPELKTDDEGPSLRNKSERRVNLSKFVESQG
EVDPEDRYVIKSNLQKLNCCLPTSANDSALPGVFIRDLDDFRKKLRFYM
VHLNDLETVLTSRPPQPASGSVSPNRGTVECGGGGSGGGGSGGGGSGGG
GSGGGS
[SEQ ID NO: 51]
SAWSHPQFEKGGGSGGGSGGSAWSHPQFEKGGGGSGGGGEARLSTTTKP
VLRTPSPVHPTGTSQPQRPEDCRPRGSVKGTGLDFACDIYIWAPLAGIC
VALLLSLIITLICYNSRRNRLLQSDYMNMTPRRPGLTRKPYQPYAPARD
FAAYRPRAKFSRSAETAANLQDPNQLYNELNLGRREEYDVLEKKRARDP
EMGGKQQRRRNPQEGVYNALQKDKMAEAYSEIGTKGERRRGKGHDGLYQ
GLSTATKDTYDALHMQTLAPRGSGQCTNYALLKLAGDVESNPGPLE

In certain embodiments, the soluble polypeptide comprises a mouse IL-7 polypeptide fused to a FLAG tagged RR12EE345L leucine zipper, a P2A peptide, an iCaspase9 polypeptide, a F2A peptide, a CD19 scFv, a myc tag, a CD8EC hinge/TM domain, a CD28 costimulatory domain, and a CD3ζ polypeptide. In certain embodiments, the soluble polypeptide comprises the amino acid sequences set forth in SEQ ID NOs: 52 and 53.

[SEQ ID NO: 52]
MFHVSFRYIFGIPPLILVLLPVTSSECHIKDKEGKAYESVLMISIDELDK
MTGTDSNCPNNEPNFERKHVCDDTKEAAFLNRAARKLKQFLKMNISEEFN
VHLLTVSQGTQTLVNCTSKEEKNVKEQKKNDACFLKRLLREIKTCWNKIL
KGSIGGGGSGGGSALGDYKDDDDKLEIRAAFLRQRNTALRTEVAELEQEV
QRLENEVSQYETRYGPLGGGKGSGRAKRATNFSLLKQAGDVEENPGPEFT
RAKRMGVQVETISPGDGRTFPKRGQTCVVHYTGMLEDGKKVDSSRDRNKP
FKFMLGKQEVIRGWEEGVAQMSVGQRAKLTISPDYAYGATGHPGIIPPHA
TLVFDVELLKLESGGGSGVDGFGDVGALESLRGNADLAYILSMEPCGHCL
IINNVNFCRESGLRTRTGSNIDCEKLRRRFSSLHFMVEVKGDLTAKKMVL
ALLELARQDHGALDCCVVVILSHGCQASHLQFPGAVYGTDGCPVSVEKIV
NIFNGTSCPSLGGKPKLFFIQACGGEQKDHGFEVASTSPEDESPGSNPEP
DATPFQEGLRTFDQLDAISSLPTPSDIFVSYSTFPGFVSWRDPKSGSWYV
ETLDDIFEQWAHSEDLQSLLLRVANAVSVKGIYKQMPGCFNFLRKKLFFK
TSVDGTGRAKRVKQTLNFDLLKLAGDVESNPGPGSLT
[SEQ ID NO: 53]
EQKLISEEDLLESSTTTKPVLRTPSPVHPTGTSQPQRPEDCRPRGSVKGT
GLDFACDIYIWAPLAGICVALLLSLIITLICYNSRRNRLLQSDYMNMTPR
RPGLTRKPYQPYAPARDFAAYRPRAKFSRSAETAANLQDPNQLYNELNLG
RREEYDVLEKKRARDPEMGGKQQRRRNPQEGVYNALQKDKMAEAYSEIGT
KGERRRGKGHDGLYQGLSTATKDTYDALHMQTLAPRGTEARL

In certain embodiments, the soluble polypeptide comprises a mouse IL-15 polypeptide fused to a FLAG tagged RR12EE345L leucine zipper, a P2A peptide, an iCaspase9 polypeptide, a F2A peptide, a CD19 scFv, a myc tag, a CD8EC hinge/TM domain, a CD28 costimulatory domain, and a CD3ζ polypeptide. In certain embodiments, the soluble polypeptide comprises the amino acid sequences set forth in SEQ ID NOs: 54 and 55.

[SEQ ID NO: 54]
MYSMQLASCVTLTLVLLVNSNWIDVRYDLEKIESLIQSIHIDTTLYTDSD
FHPSCKVTAMNCFLLELQVILHEYSNMTLNETVRNVLYLANSTLSSNKNV
AESGCKECEELEEKTFTEFLQSFIRIVQMFINTSGGGGSGGGSALGDYKD
DDDKLEIRAAFLRQRNTALRTEVAELEQEVQRLENEVSQYETRYGPLGGG
KGSGRAKRATNFSLLKQAGDVEENPGPEFTRAKRMGVQVETISPGDGRTF
PKRGQTCVVHYTGMLEDGKKVDSSRDRNKPFKFMLGKQEVIRGWEEGVAQ
MSVGQRAKLTISPDYAYGATGHPGIIPPHATLVFDVELLKLESGGGSGVD
GFGDVGALESLRGNADLAYILSMEPCGHCLIINNVNFCRESGLRTRTGSN
IDCEKLRRRFSSLHFMVEVKGDLTAKKMVLALLELARQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTSVDGTGRAKRVKQTLNFDL
LKLAGDVESNPGPGSLT
[SEQ ID NO: 55]
EQKLISEEDLLESSTTTKPVLRTPSPVHPTGTSQPQRPEDCRPRGSVKGT
GLDFACDIYIWAPLAGICVALLLSLIITLICYNSRRNRLLQSDYMNMTPR
RPGLTRKPYQPYAPARDFAAYRPRAKFSRSAETAANLQDPNQLYNELNLG
RREEYDVLEKKRARDPEMGGKQQRRRNPQEGVYNALQKDKMAEAYSEIGT
KGERRRGKGHDGLYQGLSTATKDTYDALHMQTLAPRGTEARL

In certain embodiments, the soluble polypeptide comprises a mouse IL-21 polypeptide fused to a FLAG tagged RR12EE345L leucine zipper, a P2A peptide, an iCaspase9 polypeptide, a F2A peptide, a CD19 scFv, a myc tag, a CD8EC hinge/TM domain, a CD28 costimulatory domain, and a CD3ζ polypeptide. In certain embodiments, the soluble polypeptide comprises the amino acid sequences set forth in SEQ ID NOs: 56 and 57.

[SEQ ID NO: 56]
MYSMQLASCVTLTLVLLVNSHKSSPQGPDRLLIRLRHLIDIVEQLKIYEN
DLDPELLSAPQDVKGHCEHAAFACFQKAKLKPSNPGNNKTFIIDLVAQLR
RRLPARRGGKKQKHIAKCPSCDSYEKRTPKEFLERLKWLLQKMIHQHLSG
GGGSGGGSALGDYKDDDDKLEIRAAFLRQRNTALRTEVAELEQEVQRLEN
EVSQYETRYGPLGGGKGSGRAKRATNFSLLKQAGDVEENPGPEFTRAKRM
GVQVETISPGDGRTFPKRGQTCVVHYTGMLEDGKKVDSSRDRNKPFKFML
GKQEVIRGWEEGVAQMSVGQRAKLTISPDYAYGATGHPGIIPPHATLVFD
VELLKLESGGGSGVDGFGDVGALESLRGNADLAYILSMEPCGHCLIINNV
NFCRESGLRTRTGSNIDCEKLRRRFSSLHFMVEVKGDLTAKKMVLALLEL
ARQDHGALDCCVVVILSHGCQASHLQFPGAVYGTDGCPVSVEKIVNIFNG
TSCPSLGGKPKLFFIQACGGEQKDHGFEVASTSPEDESPGSNPEPDATPF
QEGLRTFDQLDAISSLPTPSDIFVSYSTFPGFVSWRDPKSGSWYVETLDD
IFEQWAHSEDLQSLLLRVANAVSVKGIYKQMPGCFNFLRKKLFFKTSVDG
TGRAKRVKQTLNFDLLKLAGDVESNPGPGSLT
[SEQ ID NO: 57]
EQKLISEEDLLESSTTTKPVLRTPSPVHPTGTSQPQRPEDCRPRGSVKGT
GLDFACDIYIWAPLAGICVALLLSLIITLICYNSRRNRLLQSDYMNMTPR
RPGLTRKPYQPYAPARDFAAYRPRAKFSRSAETAANLQDPNQLYNELNLG
RREEYDVLEKKRARDPEMGGKQQRRRNPQEGVYNALQKDKMAEAYSEIGT
KGERRRGKGHDGLYQGLSTATKDTYDALHMQTLAPRGTEARL

In certain embodiments, the soluble polypeptide comprises a RQR sequence (having two CD20 mimotopes, and a CD34 epitope), a linker, and a RR12EE345L leucine zipper. In certain embodiments, the soluble polypeptide comprises the amino acid sequence set forth in SEQ ID NO: 134, which is provided below.

[SEQ ID NO: 134]
METDTLLLWVLLLWVPGSTGCPYSNPSLCSGGGGSELPTQGTFSNVSTNV
SPAKPTTTACPYSNPSLCGGGGSGGGSALGLEIRAAFLRQRNTALRTEVA
ELEQEVQRLENEVSQYETRYGPLGGGK

An exemplary nucleotide sequence encoding the amino acid sequence of SEQ ID NO: 134 is set forth in SEQ ID NO: 135, which is provided below.

[SEQ ID NO: 135]
ATGGAGACAGACACACTCCTGCTATGGGTGCTGCTGCTCTGGGTTCCAGG
TTCCACAGGTTGCCCCTATTCAAACCCATCACTGTGCAGCGGCGGCGGCG
GCAGCGAGCTGCCTACCCAGGGCACCTTCAGCAACGTGAGCACCAACGTG
AGCCCTGCCAAGCCTACCACCACCGCCTGCCCTTACAGCAACCCTAGCCT
GTGCGGTGGCGGAGGAAGTGGCGGAGGAAGCGCCCTAGGGCTGGAGATCC
GCGCTGCATTTTTGCGACAAAGAAATACCGCTTTGCGAACCGAAGTGGCC
GAGTTGGAGCAGGAAGTACAACGCCTGGAAAATGAAGTCAGCCAATACGA
AACTCGATATGGACCTCTCGGAGGTGGAAAG

In certain embodiments, the soluble polypeptide comprises a Q2 sequence (having two CD34 epitopes), a linker, and a RR12EE345L leucine zipper. In certain embodiments, the soluble polypeptide comprises the amino acid sequence set forth in SEQ ID NO: 136, which is provided below.

[SEQ ID NO: 136]
METDTLLLWVLLLWVPGSTGGSGELPTQGTFSNVSTNVSGGGGSGGGSEL
PTQGTFSNVSTNVSGGGGSGGGSALGLEIRAAFLRQRNTALRTEVAELEQ
EVQRLENEVSQYETRYGPLGGGK

An exemplary nucleotide sequence encoding the amino acid sequence of SEQ ID NO: 136 is set forth in SEQ ID NO: 137, which is provided below.

[SEQ ID NO: 137]
ATGGAGACAGACACACTCCTGCTATGGGTGCTGCTGCTCTGGGTTCCAGG
TTCCACAGGTGGGTCTGGGGAGTTGCCCACTCAGGGCACATTTTCTAATG
TCTCCACTAACGTAAGCGGAGGAGGAGGAAGTGGAGGAGGAAGTGAGCTG
CCTACCCAGGGCACCTTCAGCAACGTGAGCACCAACGTGAGCGGTGGTGG
TGGTAGTGGTGGTGGTAGCGCCCTAGGGCTGGAGATCCGCGCTGCATTTT
TGCGACAAAGAAATACCGCTTTGCGAACCGAAGTGGCCGAGTTGGAGCAG
GAAGTACAACGCCTGGAAAATGAAGTCAGCCAATACGAAACTCGATATGG
ACCTCTCGGAGGTGGAAAG

In certain embodiments, the membrane-bound polypeptide comprises an R2 sequence (having two CD20 mimotopes), a linker, an EE12RR345L leucine zipper, a CD28-9C hinge, a CD28 transmembrane domain, and a truncated CD3 (delta). In certain embodiments, the membrane-bound polypeptide comprises the amino acid sequence set forth in SEQ ID NO: 138, which is provided below.

[SEQ ID NO: 138]
METDTLLLWVLLLWVPGSTGCPYSNPSLCSGGGGSSGGGGSDCPYSNPSL
CGGGGSGGGSLEIEAAFLERENTALETRVAELRQRVQRLRNRVSQYRTRY
GPLGGGKGSGCHTQSSPKLEFTFWALVVVAGVLFCYGLLVTVALCVIWTR
AKFSRSAETAANLQDTFDALHMQTLAPR

An exemplary nucleotide sequence encoding the amino acid sequence of SEQ ID NO: 138 is set forth in SEQ ID NO: 139, which is provided below.

[SEQ ID NO: 139]
ATGGAGACAGACACACTCCTGCTATGGGTGCTGCTGCTCTGGGTTCCAGG
TTCCACAGGTTGCCCCTATTCAAACCCATCACTGTGCAGCGGCGGCGGCG
GCAGCAGCGGCGGGGGTGGCAGCGACTGCCCTTACAGCAACCCTAGCCTG
TGCGGTGGCGGAGGAAGTGGCGGAGGAAGCCTGGAGATTGAGGCTGCATT
TCTTGAAAGAGAAAACACTGCTCTTGAGACAAGAGTCGCCGAACTGAGGC
AGCGCGTTCAGCGCCTGCGGAACCGAGTATCTCAATACAGGACTCGGTAC
GGACCACTGGGGGGCGGTAAGGGATCTGGTTGTCATACTCAGTCATCTCC
TAAGCTGGAATTCACCTTTTGGGCACTGGTCGTGGTTGCTGGAGTCCTGT
TTTGTTATGGCTTGCTAGTGACAGTGGCTCTTTGTGTTATCTGGACAAGA
GCTAAGTTCAGCAGATCAGCCGAGACTGCCGCAAACCTTCAGGATACATT
TGATGCTTTGCACATGCAAACCCTCGCACCACGC

In certain embodiments, the system comprises a soluble polypeptide (comprising an R2 sequence and an RR12EE345L leucine zipper), a linker, a membrane-bound polypeptide (comprising an EE12RR345L leucine zipper, a PD1 dominant negative molecule, a CD4 TM domain, and Truncated CD3z (delta)). In certain embodiments, the system comprises the amino acid sequence set forth in SEQ ID NO: 140, which is provided below.

[SEQ ID NO: 140]
METDTLLLWVLLLWVPGSTGCPYSNPSLCSGGGGSSGGGGSDCPYSNPSL
CGGGGSGGGSLEIRAAFLRQRNTALRTEVAELEQEVQRLENEVSQYETRY
GPLGGGKGGGGSGGGGSGGGGSGGGSLEIEAAFLERENTALETRVAELRQ
RVQRLRNRVSQYRTRYGPLGGGKEFTGSTSGSGKPGSGEGSTKGLEVPNG
PWRSLTFYPAWLTVSEGANATFTCSLSNWSEDLMLNWNRLSPSNQTEKQA
AFCNGLSQPVQDARFQIIQLPNRHDFHMNILDTRRNDSGIYLCGAISLHP
KAKIEESPGAELVVTERILETSTRYPSPSPKPEGRFQGMVFLACVLGGSF
GFLGFLGLCILCRAKFSRSAETAANLQDTFDALHMQTLAPR

An exemplary nucleotide sequence encoding the amino acid sequence of SEQ ID NO: 140 is set forth in SEQ ID NO: 141, which is provided below.

[SEQ ID NO: 141]
ATGGAGACAGACACACTCCTGCTATGGGTGCTGCTGCTCTGGGTTCCAGG
TTCCACAGGTTGCCCCTATTCAAACCCATCACTGTGCAGCGGCGGCGGCG
GCAGCAGCGGCGGGGGTGGCAGCGACTGCCCTTACAGCAACCCTAGCCTG
TGCGGTGGCGGAGGAAGtGGCGGaGGaAGCCTGGAGATCCGCGCTGCATT
TTTGCGACAAAGAAATACCGCTTTGCGAACCGAAGTGGCCGAGTTGGAGC
AGGAAGTACAACGCCTGGAAAATGAAGTCAGCCAATACGAAACTCGATAT
GGACCTCTCGGAGGTGGAAAGGGTGGAGGTGGATCAGGTGGGGGTGGATC
TGGTGGCGGTGGATCTGGAGGAGGATCGCTGGAGATTGAGGCTGCATTTC
TTGAAAGAGAAAACACTGCTCTTGAGACAAGAGTCGCCGAACTGAGGCAG
CGCGTTCAGCGCCTGCGGAACCGAGTATCTCAATACAGGACTCGGTACGG
ACCACTGGGGGGCGGTAAGGAATTCACCGGCAGCACCAGCGGCAGCGGCA
AGCCCGGCAGCGGCGAGGGCAGCACCAAGGGCCTAGAGGTCCCCAATGGG
CCCTGGAGGTCCCTCACCTTCTACCCAGCCTGGCTCACAGTGTCAGAGGG
AGCAAATGCCACCTTCACCTGCAGCTTGTCCAACTGGTCGGAGGATCTTA
TGCTGAACTGGAACCGCCTGAGTCCCAGCAACCAGACTGAAAAACAGGCC
GCCTTCTGTAATGGTTTGAGCCAACCCGTCCAGGATGCCCGCTTCCAGAT
CATACAGCTGCCCAACAGGCATGACTTCCACATGAACATCCTTGACACAC
GGCGCAATGACAGTGGCATCTACCTCTGTGGGGCCATCTCCCTGCACCCC
AAGGCAAAAATCGAGGAGAGCCCTGGAGCAGAGCTaGTGGTAACAGAGAG
AATCCTGGAGACCTCAACAAGATATCCCAGCCCCTCGCCCAAACCAGAAG
GCCGGTTTCAAGGCATGGTGTTCCTGGCCTGCGTGCTGGGCGGCAGCTTC
GGCTTCCTGGGCTTCCTGGGCCTGTGCATCCTGTGCAGAGCTAAGTTCAG
CAGATCAGCCGAGACTGCCGCAAACCTTCAGGATACATTTGATGCTTTGC
ACATGCAAACCCTCGCACCACGC

In certain embodiments, the system comprises a Kappa signal peptide, a soluble polypeptide (e.g., an RR12EE345L leucine zipper), a linker, a membrane-bound polypeptide (having an EE12RR345L leucine zipper, and a CD80 polypeptide). In certain embodiments, the system comprises the amino acid sequence set forth in SEQ ID NO: 142, which is provided below.

[SEQ ID NO: 142]
METDTLLLWVLLLWVPGSTGLEIRAAFLRQRNTALRTEVAELEQEVQRLE
NEVSQYETRYGPLGGGKGGGGSGGGGSGGGGSGGGSLETEAAFLERENTA
LETRVAELRQRVQRLRNRVSQYRTRYGPLGGGKEFTGSTSGSGKPGSGEG
STKGVDEQLSKSVKDKVLLPCRYNSPHEDESEDRIYWQKHDKVVLSVIAG
KLKVWPEYKNRTLYDNTTYSLIILGLVLSDRGTYSCVVQKKERGTYEVKH
LALVKLSIKADFSTPNITESGNPSADTKRITCFASGGFPKPRFSWLENGR
ELPGINTTISQDPESELYTISSQLDFNTTRNHTIKCLIKYGDAHVSEDFT
WEKPPEDPPDSKNTLVLFGAGFGAVITVVVIVVIIKCFCKHRSCFRRNEA
SRETNNSLTFGPEEALAEQTVFL

An exemplary nucleotide sequence encoding the amino acid sequence of SEQ ID NO: 142 is set forth in SEQ ID NO: 143, which is provided below.

[SEQ ID NO: 143]
ATGGAGACAGACACACTCCTGCTATGGGTGCTGCTGCTCTGGGTTCCAGG
TTCCACAGGTCTGGAGATCCGCGCTGCATTTTTGCGACAAAGAAATACCG
CTTTGCGAACCGAAGTGGCCGAGTTGGAGCAGGAAGTACAACGCCTGGAA
AATGAAGTCAGCCAATACGAAACTCGATATGGACCTCTCGGAGGTGGAAA
GGGTGGAGGTGGATCAGGTGGAGGTGGATCTGGTGGAGGTGGATCTGGAG
GAGGATCGCTGGAGATTGAGGCTGCATTTCTTGAAAGAGAAAACACTGCT
CTTGAGACAAGAGTCGCCGAACTGAGGCAGCGCGTTCAGCGCCTGCGGAA
CCGAGTATCTCAATACAGGACTCGGTACGGACCACTGGGGGGCGGTAAGG
AATTCACCGGCAGCACCAGCGGCAGCGGCAAGCCCGGCAGCGGCGAGGGC
AGCACCAAGGGCGTTGATGAACAACTGTCCAAGTCAGTGAAAGATAAGGT
ATTGCTGCCTTGCCGTTACAACTCTCCTCATGAAGATGAGTCTGAAGACC
GAATCTACTGGCAAAAACATGACAAAGTGGTGCTGTCTGTCATTGCTGGG
AAACTAAAAGTGTGGCCCGAGTATAAGAACCGGACTTTATATGACAACAC
TACCTACTCTCTTATCATCCTGGGCCTGGTCCTTTCAGACCGGGGCACAT
ACAGCTGTGTCGTTCAAAAGAAGGAAAGAGGAACGTATGAAGTTAAACAC
TTGGCTTTAGTAAAGTTGTCCATCAAAGCTGACTTCTCTACCCCCAACAT
AACTGAGTCTGGAAACCCATCTGCAGACACTAAAAGGATTACCTGCTTTG
CTTCCGGGGGTTTCCCAAAGCCTCGCTTCTCTTGGTTGGAAAATGGAAGA
GAATTACCTGGCATCAATACGACAATTTCCCAAGATCCTGAATCTGAATT
GTACACCATTAGTAGCCAACTAGATTTCAATACGACTCGCAACCACACCA
TTAAGTGTCTCATTAAATATGGAGATGCTCACGTGTCAGAGGACTTCACC
TGGGAAAAACCCCCAGAAGACCCTCCTGATAGCAAGAACACACTTGTGCT
CTTTGGGGCAGGATTCGGCGCAGTAATAACAGTCGTCGTCATCGTTGTCA
TCATCAAATGCTTCTGTAAGCACAGAAGCTGTTTCAGAAGAAATGAGGCA
AGCAGAGAAACAAACAACAGCCTTACCTTCGGGCCTGAAGAAGCATTAGC
TGAACAGACCGTCTTCCTT

In certain embodiments, the membrane-bound polypeptide comprises an EE12RR345L leucine zipper, a linker, and a 4-1BBL polypeptide. In certain embodiments, the membrane-bound polypeptide comprises the amino acid sequence set forth in SEQ ID NO: 144, which is provided below.

[SEQ ID NO: 144]
MDQHTLDVEDTADARHPAGTSCPSDAALLRDTGLLADAALLSDTVRPTNA
ALPTDAAYPAVNVRDREAAWPPALNFCSRHPKLYGLVALVLLLLIAACVP
IFTRTEPRPALTITTSPNLGTRENNADQVTPVSHIGCPNTTQQGSPVFAK
LLAKNQASLCNTTLNWHSQDGAGSSYLSQGLRYEEDKKELVVDSPGLYYV
FLELKLSPTFTNTGHKVQGWVSLVLQAKPQVDDFDNLALTVELFPCSMEN
KLVDRSWSQLLLLKAGHRLSVGLRAYLHGAQDAYRDWELSYPNTTSFGLF
LVKPDNPWEEFTGSTSGSGKPGSGEGSTKGLEIEAAFLERENTALETRVA
ELRQRVQRLRNRVSQYRTRYGPLGGGK

An exemplary nucleotide sequence encoding the amino acid sequence of SEQ ID NO: 144 is set forth in SEQ ID NO: 145, which is provided below.

[SEQ ID NO: 145]
ATGGACCAGCACACACTTGATGTGGAGGATACCGCGGATGCCAGACATCC
AGCAGGTACTTCGTGCCCCTCGGATGCGGCGCTCCTCAGAGATACCGGGC
TCCTCGCGGACGCTGCGCTCCTCTCAGATACTGTGCGCCCCACAAATGCC
GCGCTCCCCACGGATGCTGCCTACCCTGCGGTTAATGTTCGGGATCGCGA
GGCCGCGTGGCCGCCTGCACTGAACTTCTGTTCCCGCCACCCAAAGCTCT
ATGGCCTAGTCGCTTTGGTCTTGCTGCTTCTGATCGCCGCCTGTGTTCCT
ATCTTCACCCGCACCGAGCCTCGGCCAGCGCTCACAATCACCACCTCGCC
CAACCTGGGTACCCGAGAGAATAATGCAGACCAGGTCACCCCTGTTTCCC
ACATTGGCTGCCCCAACACTACACAACAGGGCTCTCCTGTGTTCGCCAAG
CTACTGGCTAAAAACCAAGCATCGTTGTGCAATACAACTCTGAACTGGCA
CAGCCAAGATGGAGCTGGGAGCTCATACCTATCTCAAGGTCTGAGGTACG
AAGAAGACAAAAAGGAGTTGGTGGTAGACAGTCCCGGGCTCTACTACGTA
TTTTTGGAACTGAAGCTCAGTCCAACATTCACAAACACAGGCCACAAGGT
GCAGGGCTGGGTCTCTCTTGTTTTGCAAGCAAAGCCTCAGGTAGATGACT
TTGACAACTTGGCCCTGACAGTGGAACTGTTCCCTTGCTCCATGGAGAAC
AAGTTAGTGGACCGTTCCTGGAGTCAACTGTTGCTCCTGAAGGCTGGCCA
CCGCCTCAGTGTGGGTCTGAGGGCTTATCTGCATGGAGCCCAGGATGCAT
ACAGAGACTGGGAGCTGTCTTATCCCAACACCACCAGCTTTGGACTCTTT
CTTGTGAAACCCGACAACCCATGGGAAGAATTCACCGGCAGCACCAGCGG
CAGCGGCAAGCCCGGCAGCGGCGAGGGCAGCACCAAGGGCCTGGAGATTG
AGGCTGCATTTCTTGAAAGAGAAAACACTGCTCTTGAGACAAGAGTCGCC
GAACTGAGGCAGCGCGTTCAGCGCCTGCGGAACCGAGTATCTCAATACAG
GACTCGGTACGGACCACTGGGGGGCGGTAAG

Exemplary sequences of the elements comprised in a leucine zipper construct are as follows.

Interchain linker:

[SEQ ID NO: 58]
GTGGSTGGGGSGGGGSGGGGS

Alternate Interchain Linker 1:

[SEQ ID NO: 59]
GGGGSGGGGSGGGGSGGGGSGGGS

Alternate Interchain Linker 2:

[SEQ ID NO: 146]
GGGGSSGGGGSD

Alternate Interchain Linker 3:

[SEQ ID NO: 118]
GGGGSGGGS

Alternate Interchain Linker 4:

[SEQ ID NO: 147]
GSTSGSGKPGSGEGSTKG

Alternate Interchain Linker 5:

[SEQ ID NO: 148]
EFTGSTSGSGKPGSGEGSTKG

Alternate Interchain Linker 6:

[SEQ ID NO: 149]
GGGGSGGGSALG

Mouse IL-3 sequence used in cytokine-based receptor binding region:

[SEQ ID NO: 60]
ASISGRDTHRLTRTLNCSSIVKETIGKLPEPELKTDDEGPSLRNKSERRV
NLSKFVESQGEVDPEDRYVIKSNLQKLNCCLPTSANDSALPGVFIRDLDD
ERKKLREYMVHLNDLETVLTSRPPQPASGSVSPNRGTVEC

Human IL-3 sequence to be used in a similar IL-3 based CAR:

[SEQ ID NO: 61]
APMTQTTPLKTSWVNCSNMIDETITHLKQPPLPLLDENNLNGEDQDILME
NNLRRPNLEAFNRAVKSLQNASAIESILKNLLPCLPLATAAPTRHPIHIK
DGDWNEFRRKLTFYLKTLENAQAQQTTLSLAIF

Mouse CD8 spacer for CARs, also a non-blocking spacer for ZipR-CAR:

[SEQ ID NO: 62]
STTTKPVLRTPSPVHPTGTSQPQRPEDCRPRGSVKGTGLDFACD

Human CD8 spacer equivalent sequence:

[SEQ ID NO: 63]
TTTPAPRPPTPAPTIASQPLSLRPEACRPAAGGAVHTRGLDFACD

Mouse CD28 long spacer for CARs:

[SEQ ID NO: 64]
IEFMYPPPYLDNERSNGTIIHIKEKHLCHTQSSPKL

Human CD28 long spacer equivalent sequence:

[SEQ ID NO: 65]
IEVMYPPPYLDNEKSNGTIIHVKGKHLCPSPLFPGPSKP

Mouse Thy1.1 (CD90) spacer (non-blocking):

[SEQ ID NO: 66]
QKVTSLTACLVNQNLRLDCRHENNTKDNSIQHEFSLTREKRKHVLSGTLG
IPEHTYRSRVTLSNQPYIKVLTLANFTTKDEGDYFCELRVSGANPMSSNK
SISVYRDKLVKC

Mouse Thy1.1 (CD90) pro-peptide (used as part of Thy1.1/CD90 spacer to direct construct to the cell membrane, immediately follows the Thy1.1 spacer sequence):

[SEQ ID NO: 67]
GGISLLVQNTSWMLLLLLSLSLLQALDFISL

Human CD90 spacer—presumed to be non-blocking based on homology to mouse (Thy1.1/Thy1.2 allele variants are limited to mice):

[SEQ ID NO: 68]
QKVTSLTACLVDQSLRLDCRHENTSSSPIQYEFSLTRETKKHVLEGTVG
VPEHTYRSRTNFTSKYNMKVLYLSAFTSKDEGTYTCALHHSGHSPPISS
QNVTVLRDKLVKC

Human CD90 pro-peptide (used as part of CD90 spacer to direct construct to the cell membrane, immediately follows the CD90 spacer sequence):

[SEQ ID NO: 69]
EGISLLAQNTSWLLLLLLSLSLLQATDFMSL

Human EGFRt spacer (non-blocking):

[SEQ ID NO: 70]
RKVCNGIGIGEFKDSLSINATNIKHFKNCTSISGDLHILPVAFRGDSFTH
IPPLDPQELDILKTVKEITGFLLIQAWPENRIDLHAFENLEIIRGRIKQH
GQFSLAVVSLNITSLGLRSLKEISDGDVIISGNKNLCYANTINWKKLFGT
SGQKTKIISNRGENSCKATGQVCHALCSPEGCWGPEPRDCVSCRNVSRGR
ECVDKCNLLEGEPREFVENSECIQCHPECLPQAMNITCTGRGPDNCIQCA
HYIDGPHCVKTCPAGVMGENNTLVWKYADAGHVCHLCHPNCTYGCTGPGL
EGCPINGPKIPS

Human EGFRt transmembrane domain:

[SEQ ID NO: 71]
IATGMVGALLLLLVVALGIGLFM

Mouse CD2 transmembrane domain and short intracellular region:

[SEQ ID NO: 72]
FYVTVGVGAGGLLLVLLVALFIFCICKRRK (transmembrane
sequence underlined)

This element can promote weaker construct expression compared with CD28 transmembrane+CD3 zeta, and can be used to control expression of the leucine zipper density on cell surface. KRKK (SEQ ID NO: 150) may function as endoplasmic retention signal.

Human CD2 transmembrane domain and short intracellular region:

[SEQ ID NO: 73]
IYLIIGICGGGSLLMVFVALLVFYITKRKK (transmembrane
sequence underlined)

Mouse MHC class I transmembrane domain (H2-Kd) and short cytoplasmic linker:

[SEQ ID NO: 74]
VIIAVLVVLGAAIVTGAVVAFVMKGSG

Mouse IL-7 sequence including signal peptide:

[SEQ ID NO: 75]
MFHVSFRYIEGIPPLILVLLPVTSSECHIKDKEGKAYESVLMISIDELD
KMTGTDSNCPNNEPNEFRKHVCDDTKEAAFLNRAARKLKQFLKMNISEE
FNVHLLTVSQGTQTLVNCTSKEEKNVKEQKKNDACFLKRLLREIKTCWN
KILKGSI

Mouse IL-15 sequence+mouse IL-2 signal peptide:

[SEQ ID NO: 76]
MYSMQLASCVTLTLVLLVNSNWIDVRYDLEKIESLIQSIHIDTTLYTDS
DFHPSCKVTAMNCFLLELQVILHEYSNMTLNETVRNVLYLANSTLSSNK
NVAESGCKECEELEEKTFTEFLQSFIRIVQMFINTS

Mouse IL-21 sequence+mouse IL-2 signal peptide:

[SEQ ID NO: 77]
MYSMQLASCVTLTLVLLVNSHKSSPQGPDRLLIRLRHLIDIVEQLKIYE
NDLDPELLSAPQDVKGHCEHAAFACFQKAKLKPSNPGNNKTFIIDLVAQ
LRRRLPARRGGKKQKHIAKCPSCDSYEKRTPKEFLERLKWLLQKMIHQH
LS

Leucine Zipper linker+FLAG tag+RR12EE345L (sequence follows cytokine sequences listed above):

[SEQ ID NO: 78]
GGGGSGGGSALGDYKDDDDKLEIRAAFLRQRNTALRTEVAELEQEVQRL
ENEVSQYETRYGPLGGGK

2A peptide sequences:

| | |
|---|---|
| E2A | QCTNYALLKLAGDVESNPGP [SEQ ID NO: 79] |
| F2A | VKQTLNFDLLKLAGDVESNPGP [SEQ ID NO: 80] |
| P2A | ATNFSLLKQAGDVEENPGP [SEQ ID NO: 81] |
| T2A | EGRGSLLTCGDVEENPGP [SEQ ID NO: 82] |

Tag sequences:

| | |
|---|---|
| FLAG | DYKDDDDK [SEQ ID NO: 83] |
| Myc | EQKLISEEDL [SEQ ID NO: 84] |
| Twin | SAWSHPQFEKGGGSGGGSGGSAWSHPQFEKGGGGSG |
| Streptag-II | GGS [SEQ ID NO: 85] |
| (additional | |
| linker | |
| underlined) | |
| V5 | GKPIPNPLLGLDSTGGGGSGGGS [SEQ ID |
| (additional | NO: 86] |
| linker | |
| underlined) | |
| CD34 epitope | ELPTQGTSNVSTNVS [SEQ ID NO: 117] |
| tag | |

Signal peptide sequences:

| | |
|---|---|
| Mouse kappa | METDTLLLWVLLLWVPGSTG [SEQ ID NO: 87] |
| leader | |
| Mouse CD8 | MASPLTRFLSLNLLLLGESIILGSGEA |
| alpha | [SEQ ID NO: 88] |
| Mouse IL-2 | MYSMQLASCVTLTLVLLVNS [SEQ ID NO: 89] |
| Mouse IL-3 | MVLASSITSIHTMLLLLLMLFHLGLQ |
| | [SEQ ID NO: 90] |
| Mouse IL-7 | MFHVSFRYIFGIPPLILVLLPVISS |
| | [SEQ ID NO: 91] |
| Mouse IL-21 | MERTLVCLVVIFLGTVA [SEQ ID NO: 92] |

Additional elements:

| | |
|---|---|
| iCaspase9 | MGVQVETISPGDGRTFPKRGQTCVVHYTGMLEDGKKVDSSRDRNKPFKFMLGKQEVIRGWE |
| (Addgene | EGVAQMSVGQRAKLTISPDYAYGATGHPGIIPPHATLVEDVELLKLESGGGSGVDGEGDVG |
| 15567) | ALESLRGNADLAYILSMEPCGHCLIINNVNFCRESGLRTRTGSNIDCEKLRRRFSSLHFMV |
| | EVKGDLTAKKMVLALLELARQDHGALDCCVVVILSHGCQASHLQFPGAVYGTDGCPVSVEK |
| | IVNIENGTSCPSLGGKPKLFFIQACGGEQKDHGFEVASTSPEDESPGSNPEPDATPFQEGL |
| | RTFDQLDAISSLPTPSDIFVSYSTFPGFVSWRDPKSGSWYVETLDDIFEQWAHSEDLQSLL |
| | LRVANAVSVKGIYKQMPGCFNFLRKKLFFKTSVD [SEQ ID NO: 93] |
| tagBFP | MSELIKENMHMKLYMEGTVDNHHFKCTSEGEGKPYEGTQTMRIKVVEGGPLPFAFDILATS |
| (Addgene | FLYGSKTFINHTQGIPDFFKQSFPEGFTWERVTTYEDGGVLTATQDTSLQDGCLIYNVKIR |
| 11240) | GVNFTSNGPVMQKKTLGWEAFTETLYPADGGLEGRNDMALKLVGGSHLIANIKTTYRSKKP |
| | AKNLKMPGVYYVDYRLERIKEANNETYVEQHEVAVARYCDLPSKLGHKLN [SEQ ID NO: |
| | 94] |
| Mouse IL-3 | MVLASSTTSIHTMLLLLLMLFHLGLQASISGRDTHRLTRTLNCSSIVKETIGKLPEPELKT |
| (mature | DDEGPSLRNKSFRRVNLSKFVESQGEVDPEDRYVIKSNLQKLNCCLPTSANDSALPGVFIR |
| peptide | DLDDFRKKLRFYMVHLNDLETVLTSRPPQPASGSVSPNRGTVEC [SEQ ID NO: 95] |
| underlined, | |
| Uniprot | |
| P01586) | |
| Mouse IL-18 | MAAMSEDSCVNFKEMMFIDNTLYFIPEENGDLESDNFGRLHCTTAVIRNINDQVLFVDKRQ |
| (mature | PVFEDMTDIDQSASEPQTRLITYMYKDSEVRGLAVTLSVKDSKMSTLSCKNKIISFEEMDP |
| peptide | PENIDDIQSDLIFFQKRVPGHNKMEFESSLYEGHFLACQKEDDAFKLILKKKDENGDKSVM |
| underlined, | FTLTNLHQS [SEQ ID NO: 96] |
| uniprot | |
| P70380) | |

### 4. Methods of Use

The presently disclosed subject matter provides methods of isolating a cell comprising at least two expression vectors. In certain embodiments, the method comprises:

a) expressing in a cell i) a presently disclosed membrane-bound polypeptide encoded by a first expression vector, and ii) a presently disclosed soluble polypeptide encoded by a second expression vector,

b) contacting the cell with a substrate that binds to the tag, and

c) isolating the cell that binds to the substrate.

In certain embodiments, the method of isolating a cell comprising at least two expression vectors, comprises:

a) expressing in a cell i) a membrane-bound polypeptide encoded by a first expression vector, comprising a transmembrane domain and an extracellular domain, wherein the extracellular domain comprises a first dimerization domain and a blocking spacer, and ii) a soluble polypeptide encoded by a second expression vector, comprising a tag and a second dimerization domain, wherein both of the first and second dimerization domains comprise a leucine zipper domain, and wherein the blocking spacer prevents dimerization of the membrane-bound polypeptide with the soluble polypeptide when the membrane-bound polypeptide and the soluble polypeptide are not expressed from the same cell,

b) contacting the cell with a substrate that binds to the tag, and

c) isolating a cell that binds to the substrate.

Furthermore, the presently disclosed subject matter provides methods of sorting a plurality of cells comprising at least two vectors. In certain embodiments, the method comprises:

a) transfecting a plurality of cells with i) a first expression vector encoding a membrane-bound polypeptide disclosed herein, and ii) a second expression vector encoding a soluble polypeptide disclosed herein,

b) contacting the cells with a substrate that binds to the tag, and

c) isolating one or more cells that bind to the substrate.

In certain embodiments, step c), e.g., the step of isolating one or more cells that bind to the substrate, is preceded by step d), e.g., washing the substrate to remove cells that do not bind to the substrate.

In certain embodiments, the method of sorting a plurality of cells comprising at least two expression vectors comprises:

a) transfecting a plurality of cells with i) a first expression vector encoding a membrane-bound polypeptide comprising a transmembrane domain and an extracellular domain that comprises a first dimerization domain, and ii) a second expression vector encoding a soluble polypeptide comprising a tag and a second dimerization domain that is capable of dimerizing with the first dimerization domain, wherein each of the first and second dimerization domains comprises a leucine zipper domain, and wherein the membrane-bound polypeptide does not dimerize with the soluble polypeptide when the membrane-bound polypeptide and the soluble polypeptide are not expressed from the same cell,

b) contacting the cells with a substrate that binds to the tag, and

c) isolating one or more cells that bind to the substrate.

In certain embodiments, the soluble polypeptide comprises a tag and a third dimerization domain that is capable of dimerizing with the first dimerization domain comprised in the membrane-bound polypeptide. In certain embodiments, the third dimerization domain is capable of dimerizing with the first dimerization domain prior to dimerization between the first dimerization domain and the second dimerization domain. In certain embodiments, the third dimerization domain is capable of dimerizing with the first dimerization domain in the endoplasmic reticulum. In certain embodiments, the soluble polypeptide and the membrane-bound polypeptide are capable of forming a dimer when expressed from the same cell. In certain embodiments, the soluble polypeptide and the membrane-bound polypeptide are capable of forming a dimer in the endoplasmic reticulum, when expressed from the same cell. In certain embodiments, the soluble polypeptide and the membrane-bound polypeptide are not capable of forming a dimer when expressed from different cells due to the dimerization between the first dimerization domain and the second dimerization domain. In certain embodiments, step c), e.g., the step of isolating one or more cells that bind to the substrate, is preceded by step d), washing the substrate to remove cells that do not bind to the substrate.

In certain embodiments, the cell is selected from the group consisting of a T cell, a Natural Killer (NK) cell, a cytotoxic T lymphocyte (CTL), a regulatory T cell, a Natural Killer T (NKT) cell, a human embryonic stem cell, and a pluripotent stem cell from which lymphoid cells may be differentiated. In certain embodiments, the cell is a T cell. In certain embodiments, the cell is autologous. In certain embodiments, the leucine zippers are orthogonal zippers. In certain embodiments, the orthogonal zippers are RR/EE zippers, Fos/Jun zippers or Fos/synZip zippers. Examples of synZip-9, Fos and Jun zippers are set forth in SEQ ID NOs: 4, 5 and 6, respectively.

### 5. Cells

The presently disclosed subject matter provides cells comprising a membrane-bound polypeptide, a soluble polypeptide and/or a system disclosed herein. In certain embodiments, the polypeptides and/or the systems are capable of activating or inhibiting an immunoresponsive cell. In certain embodiments, the polypeptides and/or the systems are capable of promoting an anti-tumor effect of an immunoresponsive cell. The cells can be transduced with the polypeptides and/or the system such that the cells co-express the polypeptides and/or the systems. In certain embodiments, the cell is an immunoresponsive cell. The cell can be a cell of lymphoid lineage or a cell of myeloid lineage.

Cells of the lymphoid lineage can produce antibodies, regulate the cellular immune system, detect foreign agents in the blood, and detect cells foreign to the host, and the like. Non-limiting examples of cells of the lymphoid lineage include B cells, T cells, Natural Killer (NK) cells, dendritic cells, stem cells from which lymphoid cells may be differentiated. In certain embodiments, the stem cell is a pluripotent stem cell. In certain embodiments, the pluripotent stem cell is an embryonic stem cell or an induced pluripotent stem cells.

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of any polypeptide or system disclosed herein. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a Natural killer (NK) cell. Natural killer (NK) cells can be lymphocytes that are part of cell-mediated immunity and act during the innate immune response. NK cells do not require prior activation in order to perform their cytotoxic effect on target cells.

In certain embodiments, the cells are human lymphocytes. In certain embodiments, the human lymphocytes comprise, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

In certain embodiments, the cells of the presently disclosed subject matter are cells of the myeloid lineage. In certain embodiments, the cells of the myeloid lineage comprise, without limitation, monocytes, macrophages, basophils, neutrophils, eosinophils, mast cell, erythrocyte, and thrombocytes.

The presently disclosed cells are capable of modulating the tumor microenvironment. Tumors have a microenvironment that suppresses the host immune response using any of a series of mechanisms to protect themselves from immune surveillance, recognition, and elimination. Immune suppressive factors include, but are not limited to, infiltrating regulatory CD4⁺ T cells (Tregs), myeloid-derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred modified T cells (e.g., CART cells) upon encounter with targeted tumor cells.

In certain embodiments, the presently disclosed cells have increased cell persistence. In certain embodiments, the presently disclosed cells have decreased apoptosis and/or anergy.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. Monoclonal antibodies (mAbs) are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In certain embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be distinguished from dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

### 6. Vectors

Genetic modification of an immunoresponsive cell (e.g., a T cell) can be accomplished by transducing a substantially homogeneous cell composition with a recombinant DNA construct. In certain embodiments, a retroviral vector (is employed for the introduction of the DNA construct into the cell. For example, a polynucleotide encoding any polypeptide or system disclosed herein can be cloned into a retroviral vector and expression can be driven from its endogenous promoter, from the retroviral long terminal repeat, or from a promoter specific for a target cell type of interest. In certain embodiments, the retroviral vector is a gamma-retroviral vector. In certain embodiments, the retroviral vector is a lentiviral vector. Non-viral vectors may be used as well.

For initial genetic modification of an immunoresponsive cell to include a polypeptide and/or a system disclosed herein, a retroviral vector is generally employed for transduction, however any other suitable viral vector or non-viral delivery system can be used. The polypeptides and/or the system can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits a high efficiency of infection, stable integration into the host cell genome, and durable expression of the recombinant gene product(s) (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALENs nucleases, CRISPR). Transient expression may be obtained by RNA electroporation. In certain embodiments, recombinant receptors can be introduced by a transposon-based vector. In certain embodiments, the transposon-based vector comprises a transposon (a.k.a. a transposable element). In certain embodiments, the transposon can be recognized by a transposase. In certain embodiments, the transposase is a Sleeping Beauty transposase.

The resulting cells can be grown under conditions similar to those for unmodified cells, whereby the modified cells can be expanded and used for a variety of purposes.

### 7. Polypeptides and Analogs

Also included in the presently disclosed subject matter are CD28, CD8, CD80, 4-1BBL, PD-1, and CD3ζ polypeptides, the membrane-bound polypeptide disclosed herein and the soluble polypeptide disclosed herein, or fragments thereof that are modified in ways that enhance their therapeutic efficacy when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD8, CD28, CD80, 4-1BBL, PD-1, and CD3ζ). Analogs can differ from a naturally-occurring polypeptide disclosed herein by amino acid sequence differences, by post-translational modifications, or by both. Analogs can exhibit at least about 80%, at least about 85%, at least about 90%, at least about 95%, at least about 96%, at least about 97%, at least about 98%, at least about 99% or at least about 100% homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein/peptide disclosed herein. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example 1—Leucine Zipper Cell-Sorting Affinity Tags

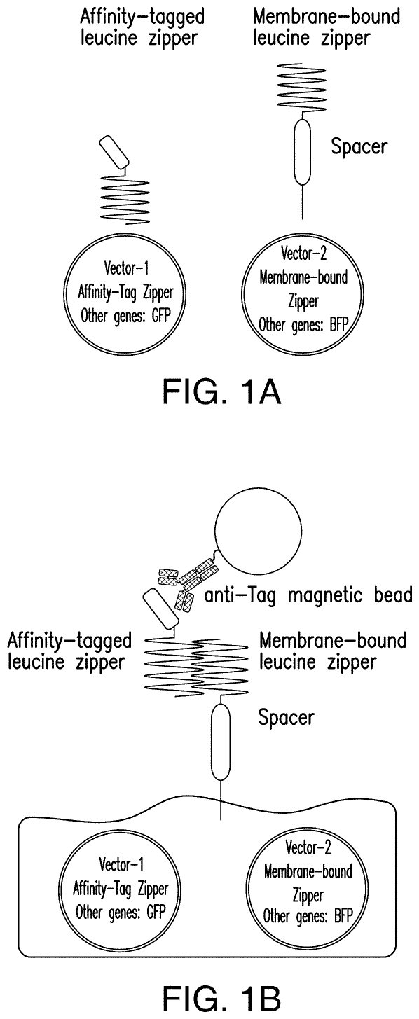

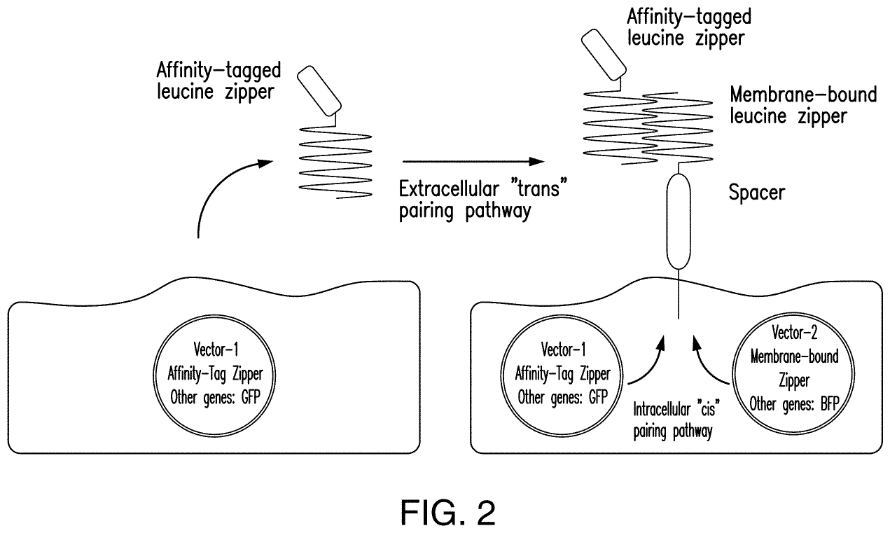

A method was developed to allow selective sorting of cells with simultaneous co-transduction with two viral vectors to allow transfer of larger amounts of genetic information without exceeding packaging limits of the viruses. To achieve this, a binary, leucine zipper-based, affinity tag method was designed to allow magnetic selection of cells co-transduced with two viral vectors with each expressing half of the leucine zipper-based affinity tag system. In this system, one viral vector encoded a leucine zipper (e.g., RR12EE345L) with a linked affinity tag (e.g. FLAG, streptag, myc, etc.) (FIG. 1). The second viral vector encoded a membrane-bound leucine zipper (e.g., EE12RR345L) with high predilection to form a heterodimer with the RR12EE345L-affinity tag zipper. When co-transduced into the same target cell, surface capture of the RR12EE345L-affinity tag by the membrane-bound EE12RR345L leucine zipper occurred, allowing surface detection of the affinity tag by flow cytometry and immuno-magnetic selection using magnetic beads conjugated to antibodies specific for the affinity tag.

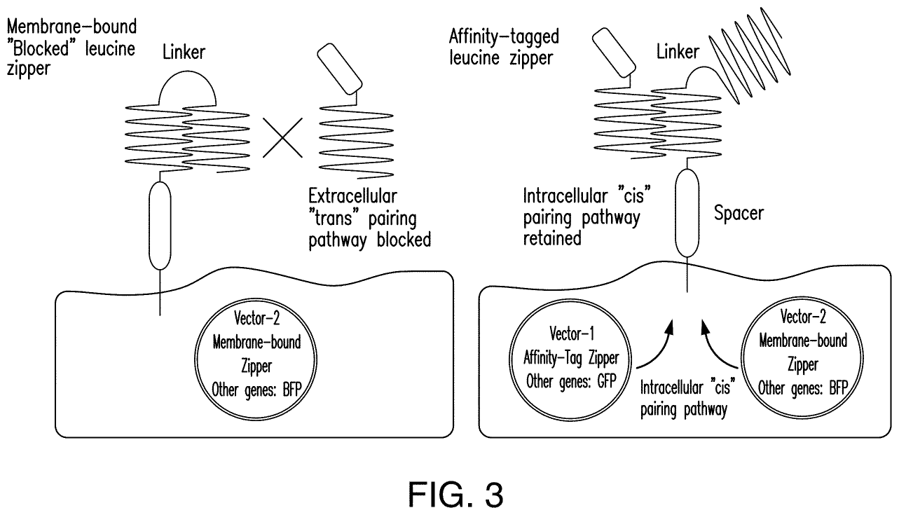

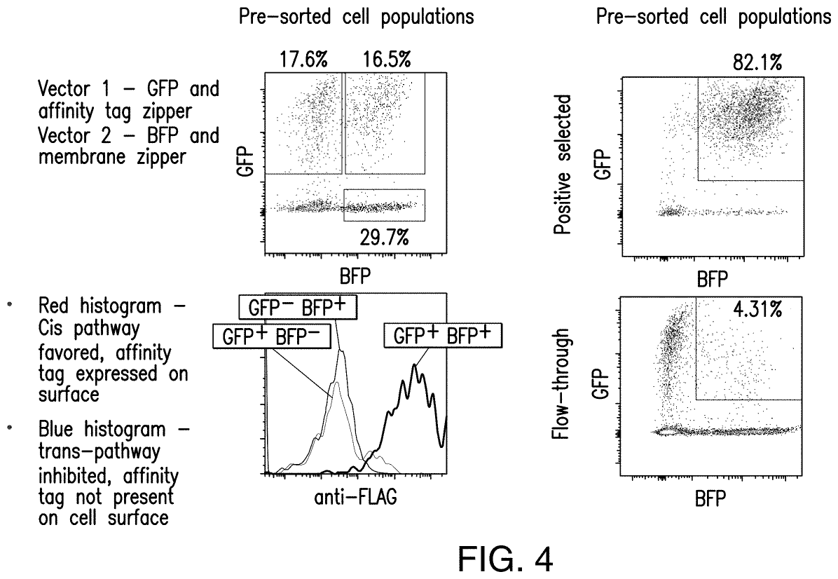

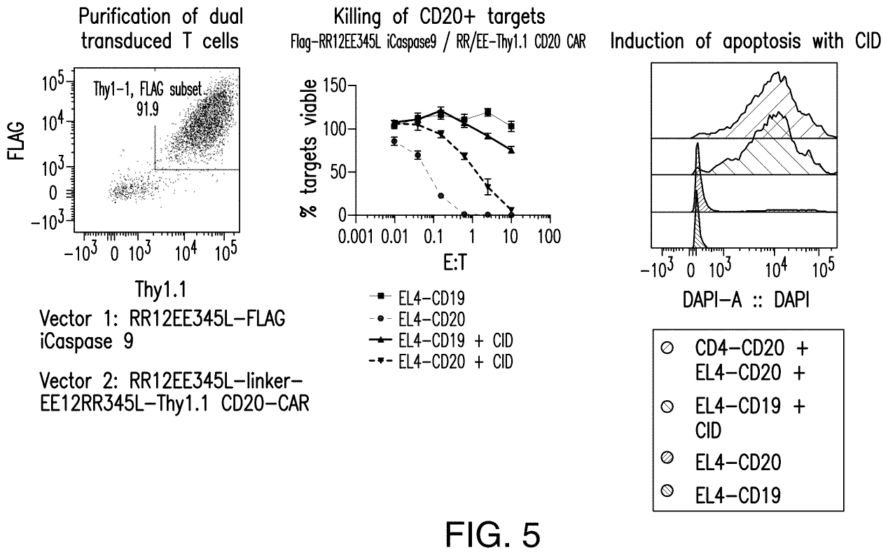

In certain circumstances, the secreted RR12EE345L-affinity tag leucine zipper was able to pair extracellularly with cells expressing only the membrane-bound EE12RR345L leucine zipper (FIG. 2). To prevent such pairing, the membrane-bound EE12RR345L leucine zipper was modified to contain a “self-blocking” feature, so that pairing of the RR12EE345L-affinity tag leucine zipper with the membrane-bound EE12RR345L leucine zipper occurred only intracellularly in cells transduced with both of the two retroviral vectors, whereas the extracellular pairing pathway was inhibited (FIGS. 3 and 4). As shown in FIG. 4, sorting of FLAG-tagged leucine zipper sorting constructs with anti-FLAG magnetic beads yielded purified population of dual-transduced primary mouse T cells. The “self-blocking” feature was achieved by generating a membrane-bound EE12RR345L leucine zipper with a linked RR12EE345L chain lacking an affinity tag. Possible genes co-expressed with the leucine zipper sorting systems include, but are not limited to: chimeric antigen receptors (CARs), costimulatory chimeric receptors (CCRs), cytokines and chemokines, suicide genes, synNotch receptors and corresponding transactivated gene constructs, and costimulatory ligands.

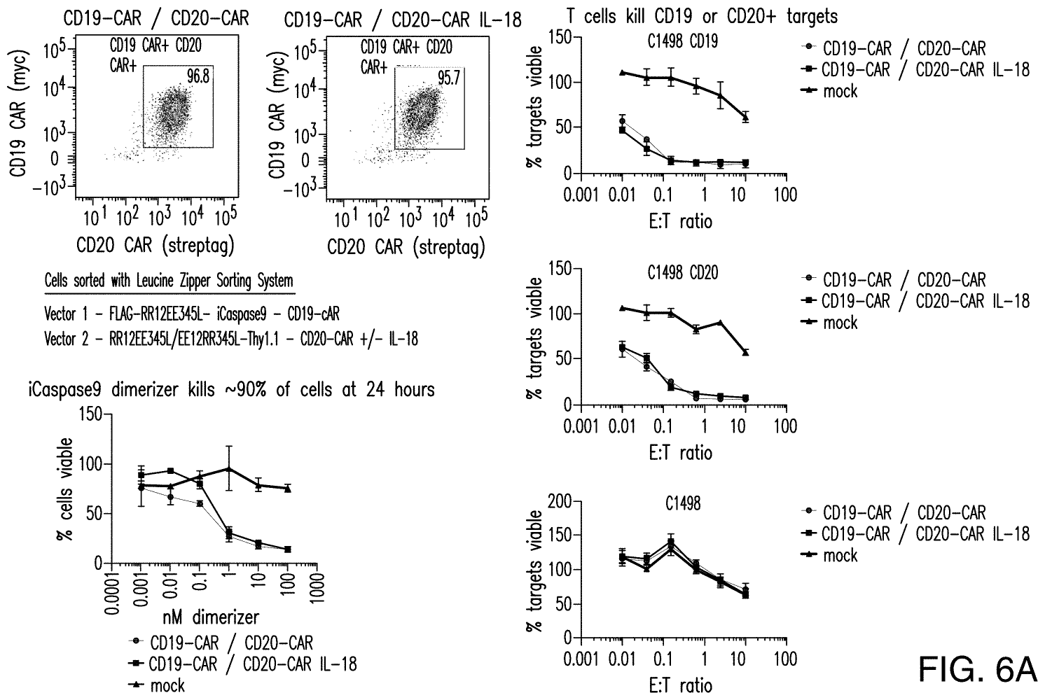

Initial investigation with an iCaspase 9 suicide gene on one vector and a CD20-CAR on the other vector demonstrated high cytolytic activity to target cells by CD20-CAR T cells and >90% induction of T cell apoptosis by activation of iCaspase 9 by the chemical inducer of dimerization (FIG. 5).

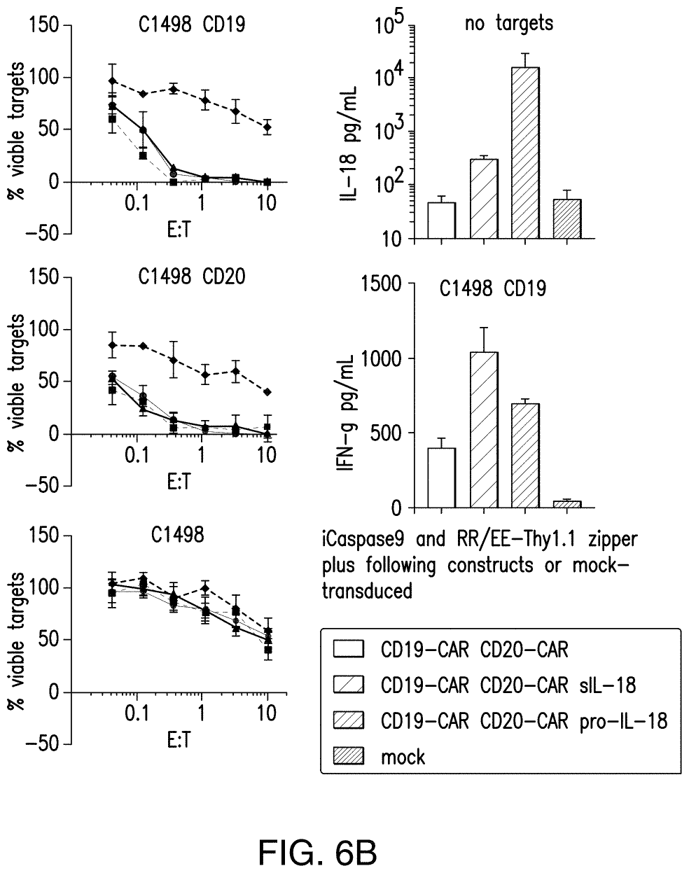

Furthermore, FIG. 6 shows that the leucine zipper sorting system were used to purify cells to above 95%, wherein the cells comprising two vectors expressing CD19-CAR, CD20-CAR and iCaspase9, and optionally IL-18. These cells were capable of killing CD19 or CD20+ targets and had enhanced cytokine expression, and incubation with an iCaspase9 dimerizer led to about 90% cell death.

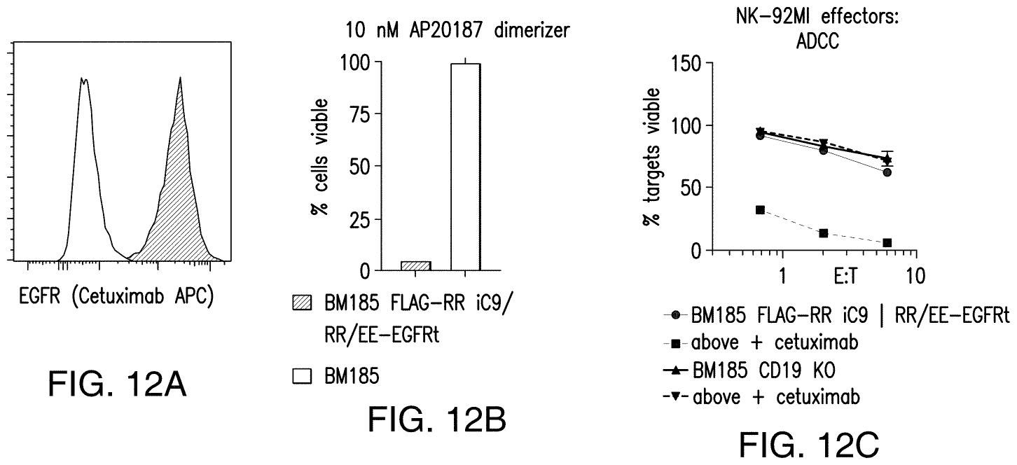

Additionally, the spacer molecule contained within the membrane bound leucine zipper can be targeted by monoclonal antibodies to permit in vivo depletion of cells expressing the constructs (FIG. 3). For mouse and human T cells, Thy1.1 and a truncated EGFR (EGFRt) molecule, respectively, can be used for the spacers to permit antibody-mediated depletion in addition to specific sorting of cells with integration of both vectors. For example, FIGS. 12A-12C demonstrate that a truncated EGFR spacer (EGFRt) fused to linker-blocked leucine zipper facilitated cell sorting and antibody dependent cell-mediated cytotoxicity (ADCC).

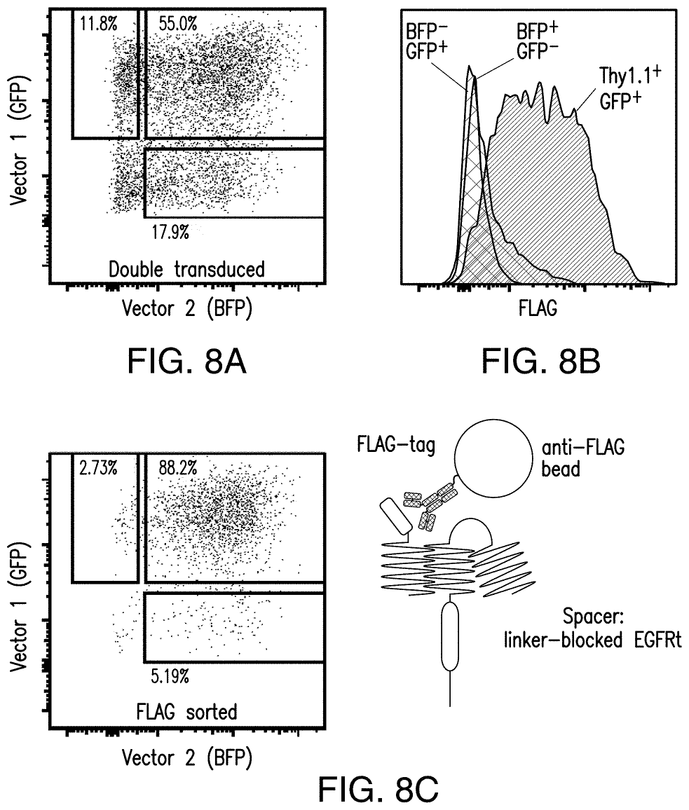

FIGS. 8A-8C further demonstrate that a linker-blocked truncated EGFR-spacer transmembrane zipper facilitated MACS sorting of dual-transduced cell population by blocking pairing between the membrane-bound leucine zipper and soluble leucine zipper expressed from different cells.

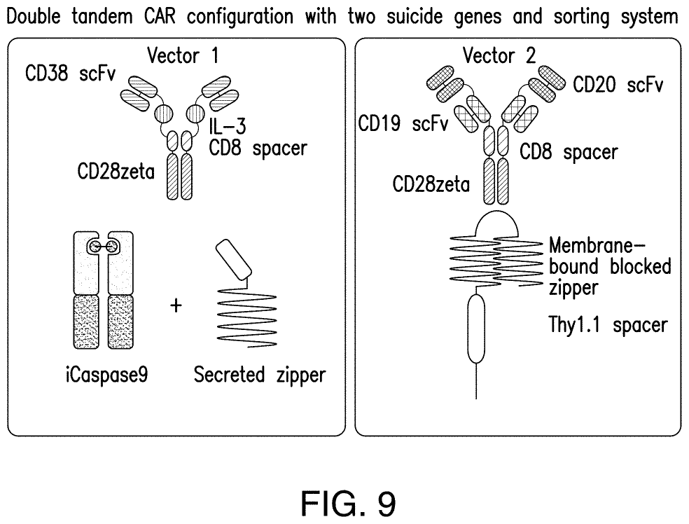

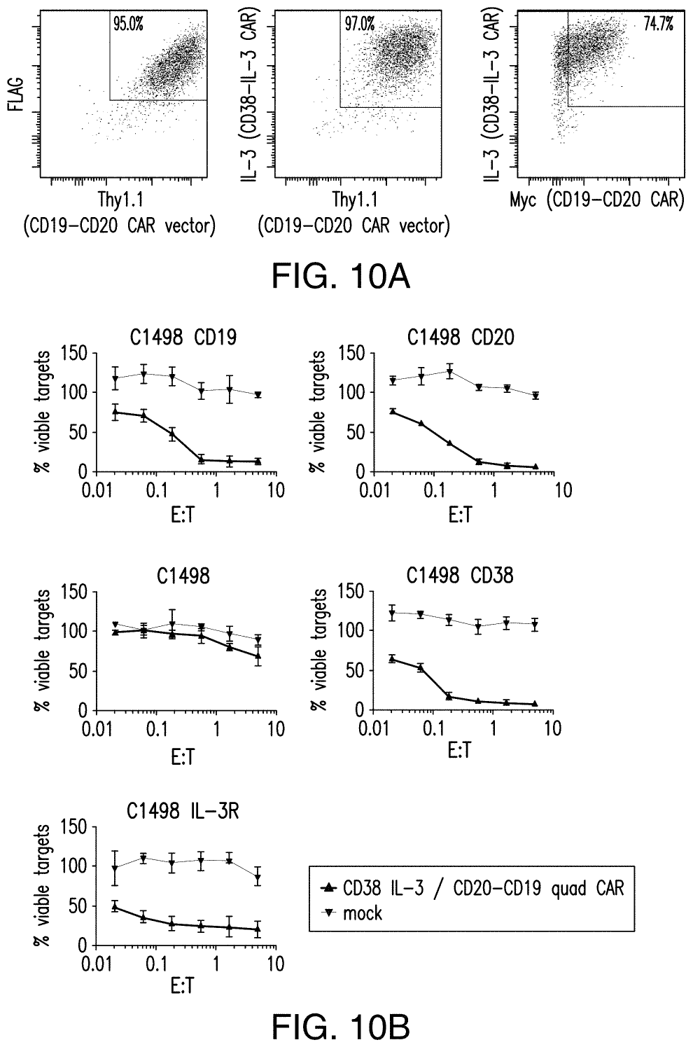

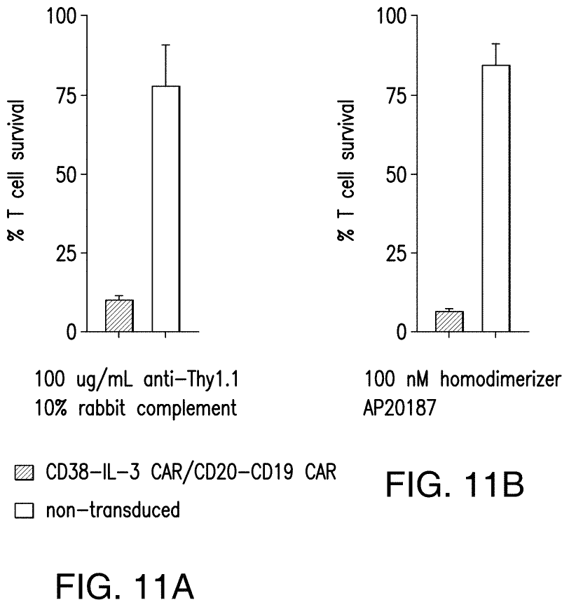

As another example of a binary system, FIG. 9 depicts double tandem CAR configuration combined with iCaspase9 and Blocked Thy1.1 Leucine Zipper Sort-Suicide Construct. Two retroviral vectors encoding Leucine Zipper Sorting System constructs and tandem CARs were used to transduce T cells. Use of same spacer/hinge like CD8 in two separate CARs expressed on the same cell can promote heterodimerization. Use of different spacer combinations (e.g. CD8 spacer/CD28 spacer) can be used to avoid heterodimerization. FIGS. 10A-10B demonstrate that the leucine Zipper Sorting System enabled single-step MACS sorting of T cells expressing double tandem CARs. FIGS. 11A-11B further demonstrate that the leucine Zipper Sorting System enabled deletion of sorted T cells using two suicide genes.

### Example 2—Leucine Zipper Cell-Sorting System Comprising Short Spacer/Hinge Region in the Membrane-Bound Polypeptide

A new design of the membrane-bound leucine zipper was developed that can inhibit the binding of soluble tagged leucine zippers secreted by other cells, but still allows binding of internally generated tagged leucine zippers, without the self-blocking feature described in Example 1. Such a membrane-bound polypeptide comprised a very small extracellular domain, which precluded antibody epitopes such as Thy1.1 or EGFRt. For example, a membrane-bound leucine zipper polypeptide comprising a CD8 spacer demonstrated binding of soluble scFv leucine zippers expressed both from the same cell that expressed the membrane-bound polypeptide and from other cells. However, a membrane-bound leucine zipper polypeptide comprising a truncated CD28 9 amino acid spacer or an IgG1 hinge only bound soluble scFv leucine zippers expressed in the same cell with the membrane-bound polypeptide.

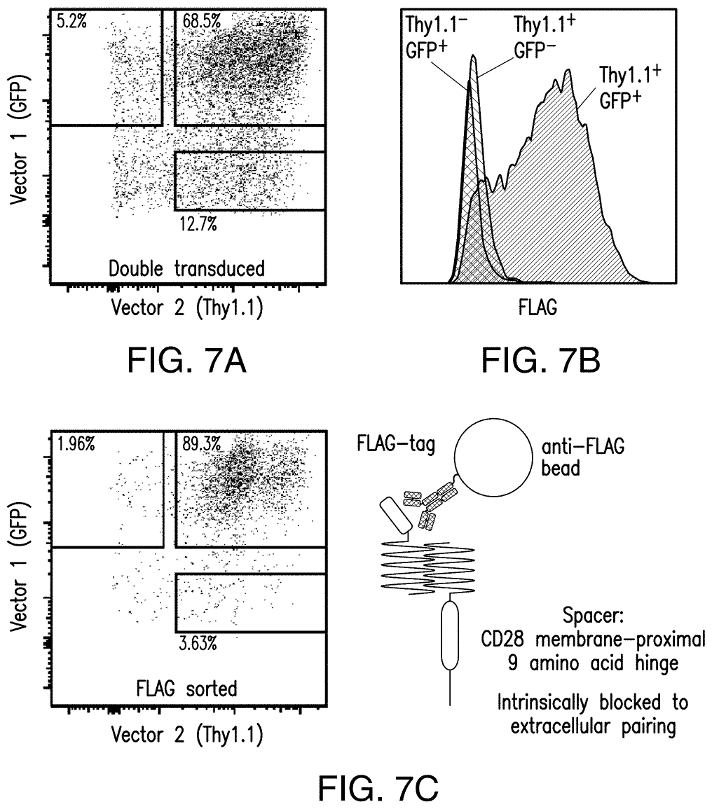

As shown in FIGS. 7A-7C, a truncated CD28 membrane proximal hinge-spacer transmembrane leucine zipper facilitated MACS sorting of dual-transduced cell population by blocking pairing between the membrane-bound leucine zipper and soluble leucine zipper expressed from different cells.

### Example 3—Leucine Zipper Cell-Sorting System Comprising Transposed Cytokine in the Soluble Polypeptide

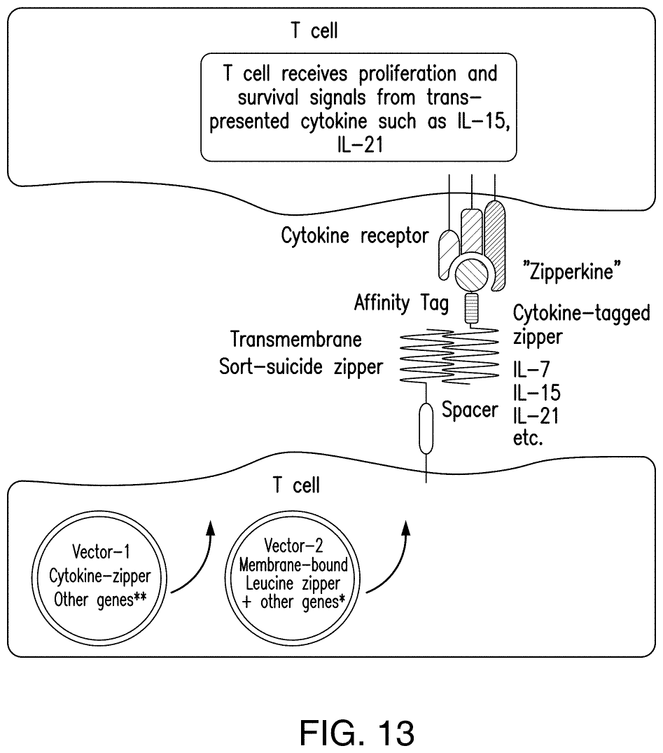

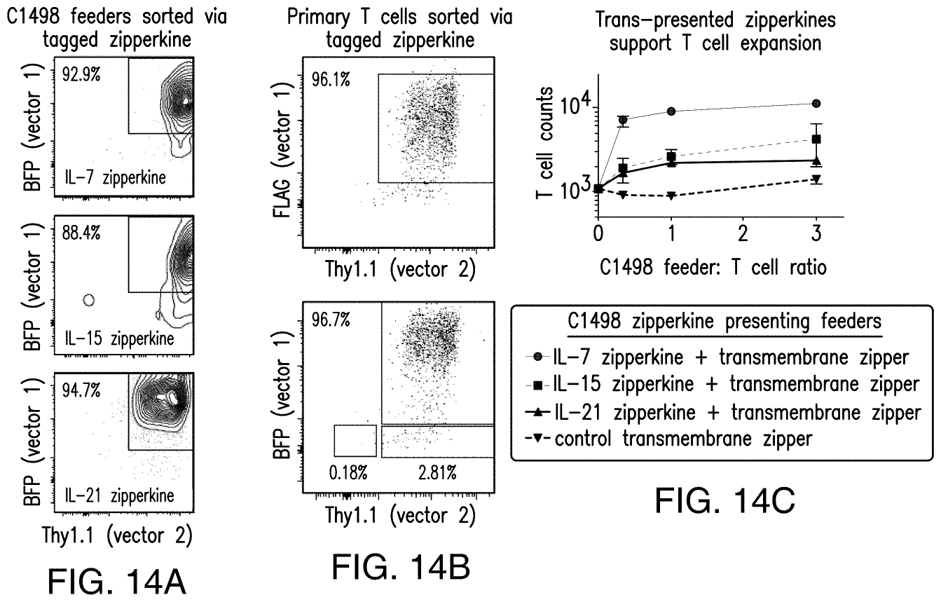

As shown in FIG. 13, cytokine-tagged zippers, “zipperkines”, were engineered to facilitate secretion and trans-presentation of cytokines while retaining sorting function of affinity-tagged secreted leucine zippers. Cytokines such as IL-7, IL-15, and IL-21 can fused to affinity tags and a heterodimerizing leucine zipper. Zipperkines were secreted to interact with cytokine receptors on T cells or co-expressed with intrinsically-blocked transmembrane leucine zipper to facilitate sorting of two vector co-transduced cells and trans-presentation of cytokines. FIGS. 14A-14C demonstrate that zipperkines retained functional sorting feature of Leucine Zipper Sorting System and promote T cell proliferation.

### Example 4—Leucine Zipper Cell-Sorting System Comprising Epitope Tag and Mimotope

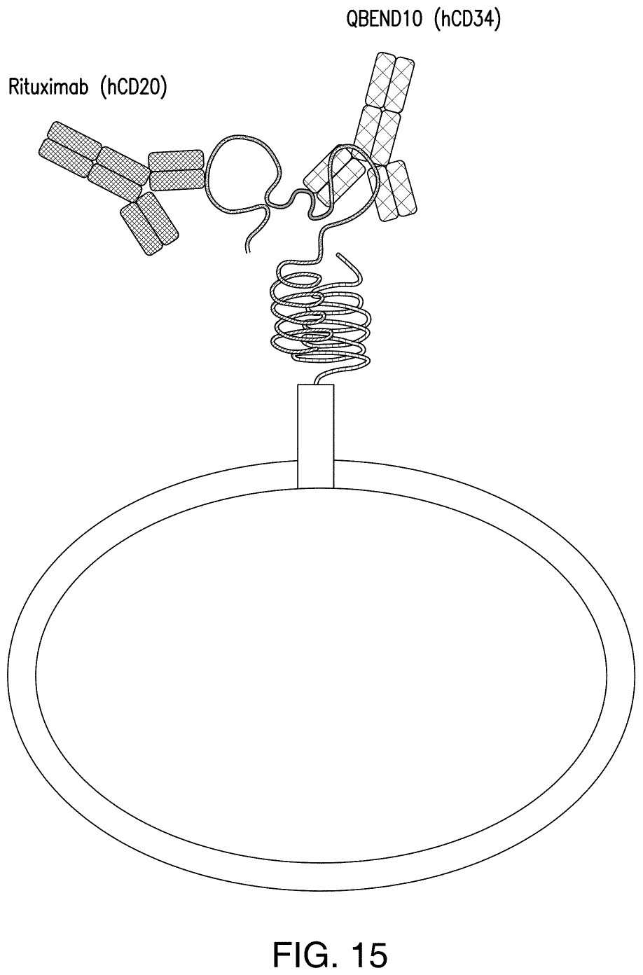

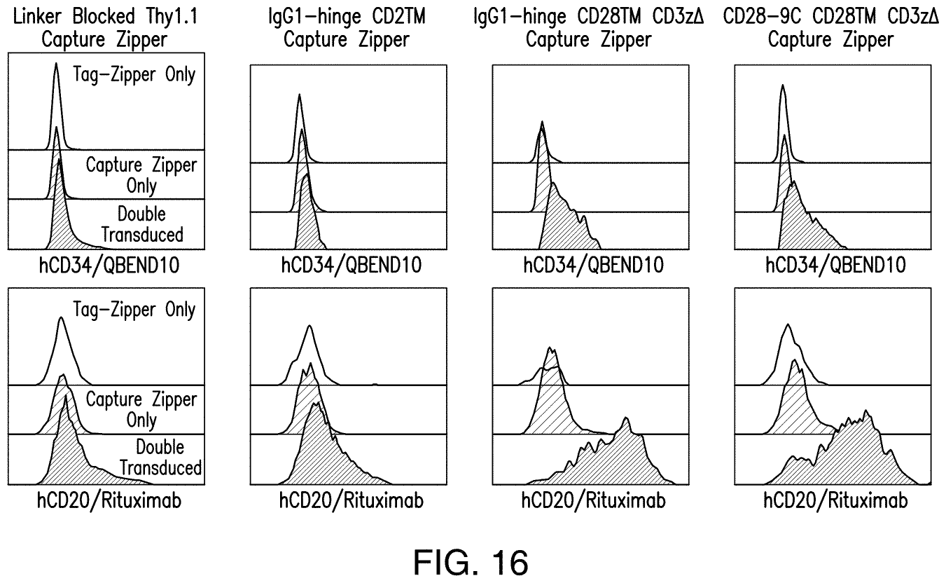

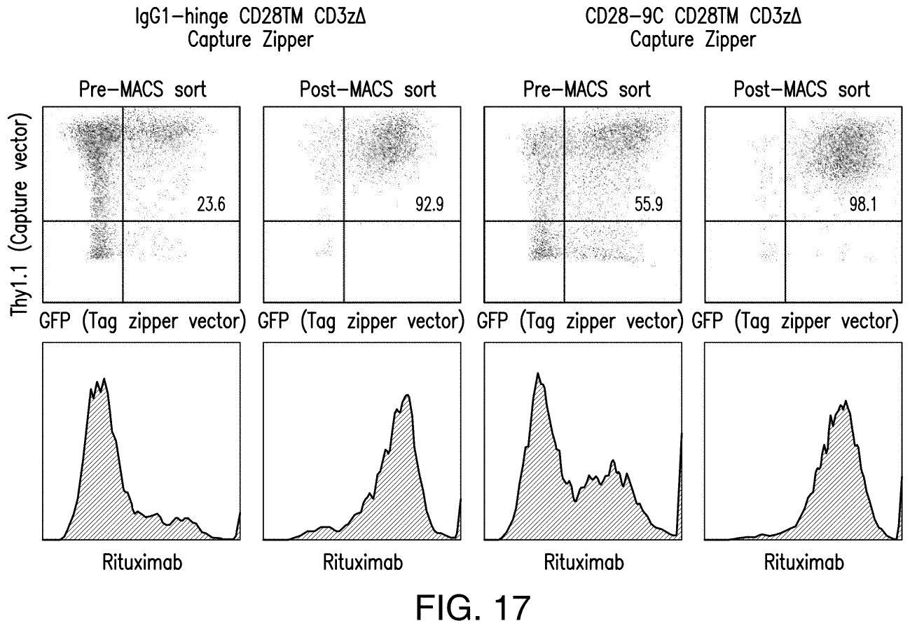

A sorting system comprising tandem two CD20 mimotopes and a CD34 epitope tag (RQR-RR12EE345L) was generated, as shown in FIG. 15. The capacity of the sorting system by using beads comprising an anti-CD34 antibody was assessed. As shown in FIG. 16, optimal CD20 mimotope CD34 leucine zipper tag capture and presentation required highly expressing truncated capture leucine zippers. Efficient CD34 and CD20 staining were observed when using the IgG1-hinge CD28TM CD3zΔ and CD28-9C CD28TM CD3zΔ capture leucine zippers. C1498 cells were double transduced with capture leucine zippers as shown in FIG. 17 and a second vector encoding the CD20 mimotope CD34 leucine zipper tag (RQR-RR12EE345L). Cells were subsequently magnetically sorted using anti-CD34 magnetic beads. As shown in FIG. 17, cells were sorted by anti-CD34 magnetic beads.

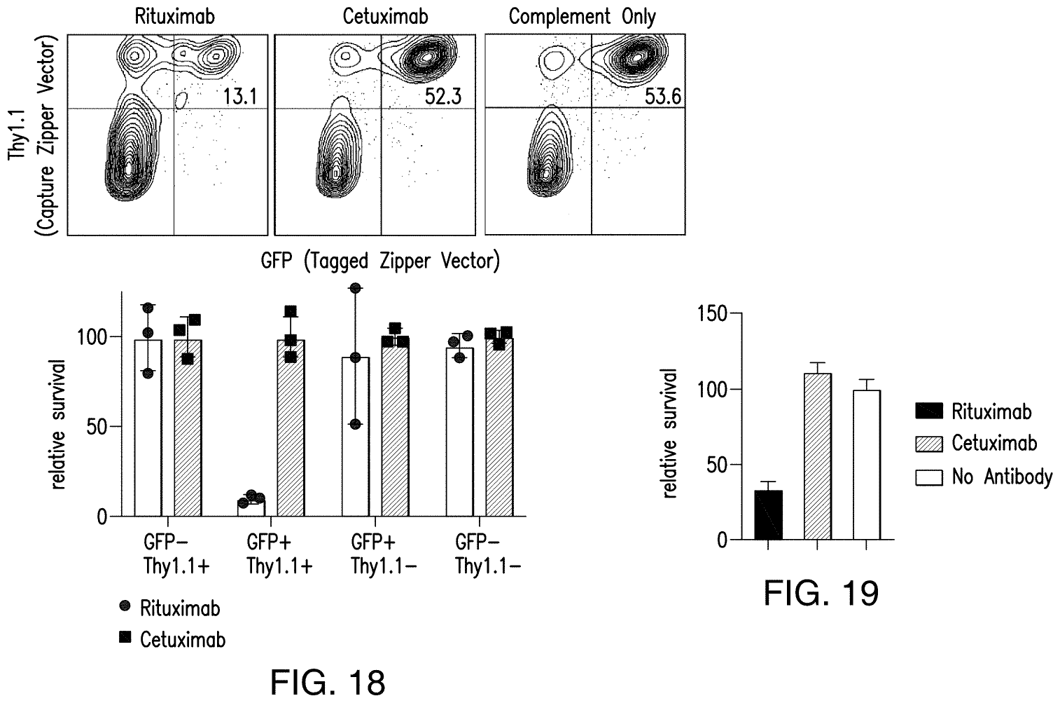

Next, C1498 cells were double transduced with capture leucine zippers as shown in FIG. 18 and a second vector encoding the tandem CD20 mimotope/CD34 leucine zipper tag (RQR-RR12EE345L). Subsequently, the cells were incubated with anti-CD20 antibody Rituximab or irrelevant antibody Cetuximab in the presence of complement. As shown in FIG. 18, only cells transduced with both RQR tagged leucine zipper and capture leucine zipper vectors are depleted by anti-CD20 antibodies. Thus, selective depletion of double transduced cells was achieved by using anti-CD20 antibody Rituximab.

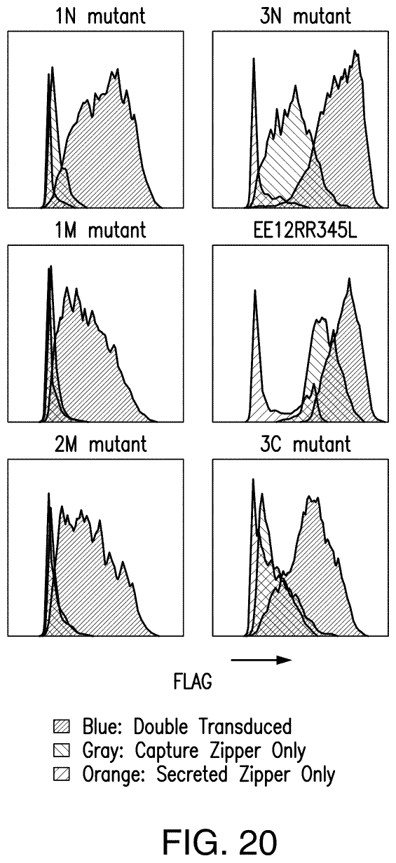

Furthermore, C1498 cells were double transduced with vectors encoding (1) tandem CD34 binding motif-tagged secreted leucine zipper (Q2-RR12EE345L) and (2) tandem circular CD20 mimotope-tagged capture leucine zipper (R2-EE12RR345L CD28-9c delta). Next, the cells were magnetically sorted with CD34 beads. Subsequently, the cells were incubated with antibodies in the presence of complement. As shown in FIG. 19, separated CD20 and CD34 binding domains enabled selective magnetic sorting and antibody-mediated depletion.

### Example 5—Leucine Zipper Cell-Sorting System Comprising Mutant Membrane-Bound Polypeptide

Mutant blocking leucine zippers increase capture and presentation of secreted leucine zippers, but can demonstrate increased extracellular paring mode (“Surface Painting”). A series of mutations were made in the “g” residues of the blocking leucine zipper to reduce heterodimerization affinity between the capture leucine zipper and the linked blocking leucine zipper. The following six mutants were made: 1N mutant: E1R2EE345L (having the amino acid sequence set forth in SEQ ID NO: 98), 1M mutant: RR123E45L (having the amino acid sequence set forth in SEQ ID NO: 99), 2N mutant: EE12345L (having the amino acid sequence set forth in SEQ ID NO: 102), 2M mutant: RR1234E5L (having the amino acid sequence set forth in SEQ ID NO: 103), 3N mutant: EE12R3E45L (having the amino acid sequence set forth in SEQ ID NO: 106), and 3C mutant: RR12345L (having the amino acid sequence set forth in SEQ ID NO: 107).

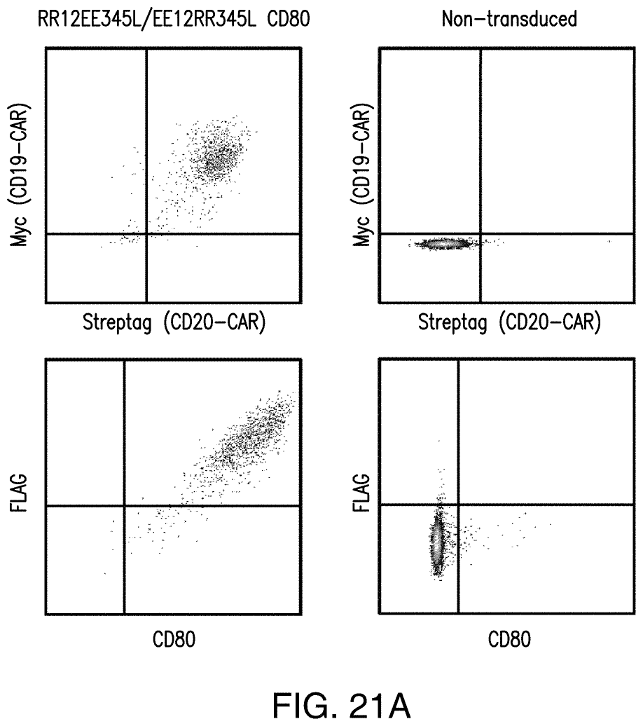

C1498 cells were co-transduced with the FLAG-RR12EE345L GFP vector and one of the six mutants as part of RR12EE345L linker EE12RR345L BFP vector constructs. As shown in FIG. 20, 3N mutant enhanced presentation of secreted leucine zipper. The 3N mutant showed increased FLAG binding in double transduced cells (intracellular pairing), but also in single-transduced capture leucine zipper-only cells (extracellular pairing). Symmetrical use of EE12RR345L for both the capture and blocking leucine zipper resulted strong FLAG-zipper capture by both single and double-transduced cells. Thus, mutating the RR12EE345L leucine zipper at “g” position residues to include repulsive amino acid interactions can reduce the degree of blocking by the linked RR12EE345L-based mutant leucine zipper.

### Example 6—Leucine Zipper Cell-Sorting System Comprising Functionalized Membrane-Bound Polypeptide

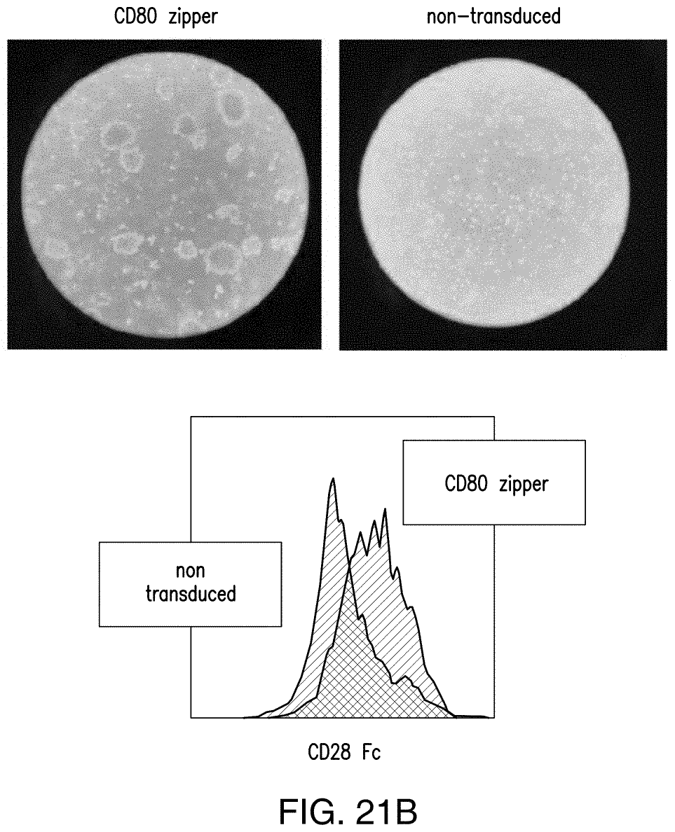

CD80 (B7-1) molecule functionalized to present blocked capture leucine zipper, permitting magnetic sorting with FLAG-RR12EE345L leucine zipper. T cells were transduced with vectors encoding (a) FLAG-RR12EE345L iCaspase9 CD19-myc-CAR and (b) RR12EE345L linker EE12RR345L CD80 CD20-streptag-CAR. Next, cells were magnetically sorted with anti-FLAG magnetic beads. As shown in FIG. 21A, sorted cells showed high purity for CD19 and CD20 CARs (Myc, Streptag, respectively) and CD80 functionalized leucine zipper. As shown in FIG. 21B, RR12EE345L linker EE12RR345L CD80 expressing T cells formed conjugates in culture and bound to soluble CD28-Fc.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or fragments thereof.

All patents and publications mentioned in this specification are herein incorporated by reference to the same extent as if each independent patent and publication was specifically and individually indicated to be incorporated by reference.

What is claimed is:

1. A membrane-bound polypeptide, comprising:
a) a transmembrane domain, and
b) an extracellular domain comprising a first dimerization domain and a second dimerization domain that is capable of dimerizing with the first dimerization domain at a cell surface,
wherein each of the first and second dimerization domains comprises a leucine zipper domain.

2. The membrane-bound polypeptide of claim 1, wherein the first dimerization domain comprises the amino acid sequence set forth in SEQ ID NO: 1, SEQ ID NO: 2, or SEQ ID NO: 97, and the second dimerization domain comprises the amino acid sequence set forth in SEQ ID NO: 1, SEQ ID NO: 2, or SEQ ID NO: 106.

3. The membrane-bound polypeptide of claim 1, wherein the extracellular domain further comprises:
a) a linker between the first dimerization domain and the second dimerization domain;
b) a spacer/hinge domain between the first dimerization domain and the transmembrane domain;
c) a co-stimulatory ligand or a fragment thereof and/or
d) a dominant negative form of a molecule or a fragment thereof.

4. The membrane-bound polypeptide of claim 3, wherein
a) the linker comprises the amino acid sequence set forth in SEQ ID NO: 3;
b) the spacer/hinge domain i) comprises an epitope recognized by an antibody, wherein binding of the antibody to the epitope mediates depletion of a cell expressing the membrane-bound polypeptide; and/or ii) comprises a Thy1.1 molecule or a truncated EGFR molecule (EGFRt);
c) the co-stimulatory ligand is selected from the group consisting of tumor necrosis factor (TNF) family members, immunoglobulin (Ig) superfamily members, and combinations thereof; and/or
d) the molecule is selected from the group consisting of inhibitors of immune checkpoint molecules, tumor necrosis factor receptor superfamily (TNFRSF) members, Transforming growth factor beta (TGFβ) receptors, and combinations thereof.

5. The membrane-bound polypeptide of claim 1, further comprising an intracellular domain.

6. The membrane-bound polypeptide of claim 5, wherein the intracellular domain comprises a CD3ζ domain, a costimulatory domain, a suicide gene, or a fragment of a combination thereof.

7. The membrane-bound polypeptide of claim 4, wherein
a) the TNF family member is selected from the group consisting of 4-1BBL, OX40L, CD70, GITRL, CD40L, CD30L, and combinations thereof; or
b) the Ig superfamily member is selected from the group consisting of CD80, CD86, ICOSLG, and combinations thereof; and/or
c) the co-stimulatory ligand is 4-1BBL or CD80;
d) the immune checkpoint molecule is selected from the group consisting of PD-1, CTLA-4, B7-H3, B7-H4, BTLA, TIM-3, LAG-3, TIGIT, LAIR1, CD200, CD200R, HVEM, 2B4, CD160, Galectin9, and combinations thereof; and/or
e) the TNFRSF member is selected from the group consisting of Fas, a Tumor necrosis factor receptor, OX40, CD40, CD27, CD30, 4-1BB, and combinations thereof.

8. The membrane-bound polypeptide of claim 1, wherein the extracellular domain further comprises a tag and/or a mimotope recognized by a second antibody.

9. The membrane-bound polypeptide of claim 8, wherein
a) the tag comprises an epitope tag recognized by a first antibody or an affinity tag that binds to a substrate;
b) binding of the second antibody to the mimotope mediates depletion of a cell comprising the membrane-bound polypeptide; and/or the mimotope is a CD20 mimotope recognized by an anti-CD20 antibody.

10. The membrane-bound polypeptide of claim 9, wherein
a) the epitope tag is selected from the group consisting of a Myc-tag, a HA-tag, a Flag-tag, a V5-tag, a T7-tag, a CD34-tag, and combinations thereof;
b) the affinity tag is selected from the group consisting of a His-tag, a Strep-tag, an E-tag, a streptavidin binding protein tag (SBP-tag), and combinations thereof; and/or
c) the anti-CD20 antibody is Rituxumab.

11. The membrane-bound polypeptide of claim 1, wherein the leucine zippers are orthogonal zippers.

12. A system for isolating a cell comprising at least two expression vectors, comprising:
a) a membrane-bound polypeptide of claim 1 encoded by a first expression vector, and
b) a soluble polypeptide encoded by a second expression vector, comprising a tag and a third dimerization domain that is capable of dimerizing with the first dimerization domain.

13. A system for isolating a cell comprising at least two expression vectors, comprising:
a) a membrane-bound polypeptide encoded by a first expression vector, comprising a transmembrane domain and an extracellular domain, wherein the extracellular domain comprises a first dimerization domain and a blocking spacer, and
b) a soluble polypeptide encoded by a second expression vector, comprising a tag and a second dimerization domain,
wherein each of the first and second dimerization domains comprises a leucine zipper domain, and wherein the blocking spacer prevents dimerization of the membrane-bound polypeptide with the soluble polypeptide when the membrane-bound polypeptide and the soluble polypeptide are not expressed from the same cell.

14. A method of isolating a cell comprising at least two expression vectors, comprising:
a) expressing in a cell
i) a membrane-bound polypeptide of claim 1 encoded by a first expression vector, and
ii) a soluble polypeptide encoded by a second expression vector, comprising a tag and a third dimerization domain that is capable of dimerizing with the first dimerization domain,
b) contacting the cell with a substrate that binds to the tag, and
c) isolating the cell that binds to the substrate.

15. A method of sorting a plurality of cells comprising at least two expression vectors, comprising:
a) transfecting a plurality of cells with
i) a first expression vector encoding a membrane-bound polypeptide of claim 1, and
ii) a second expression vector encoding a soluble polypeptide comprising a tag and a third dimerization domain that is capable of dimerizing with the first dimerization domain,
b) contacting the cells with a substrate that binds to the tag, and
c) isolating one or more cells that bind to the substrate.

16. A method of isolating a cell comprising at least two expression vectors, comprising:
a) expressing in a cell
i) a membrane-bound polypeptide encoded by a first expression vector, comprising a transmembrane domain and an extracellular domain, wherein the extracellular domain comprises a first dimerization domain and a blocking spacer, and
ii) a soluble polypeptide encoded by a second expression vector, comprising a tag and a second dimerization domain, wherein both of the first and second dimerization domains comprise a leucine zipper domain, and wherein the blocking spacer prevents dimerization of the membrane-bound polypeptide with the soluble polypeptide when the membrane-bound polypeptide and the soluble polypeptide are not expressed from the same cell,
b) contacting the cell with a substrate that binds to the tag, and
c) isolating a cell that binds to the substrate.

17. A method of sorting a plurality of cells comprising at least two expression vectors, comprising:
a) transfecting a plurality of cells with
i) a first expression vector encoding a membrane-bound polypeptide comprising a transmembrane domain and an extracellular domain that comprises a first dimerization domain, and
ii) a second expression vector encoding a soluble polypeptide comprising a tag and a second dimerization domain that is capable of dimerizing with the first dimerization domain, wherein each of the first and second dimerization domains comprises a leucine zipper domain, and wherein the membrane-bound polypeptide does not dimerize with the soluble polypeptide when the membrane-bound polypeptide and the soluble polypeptide are not expressed from the same cell,
b) contacting the cells with a substrate that binds to the tag, and
c) isolating one or more cells that bind to the substrate.

18. A nucleic acid molecule encoding the membrane-bound polypeptide of claim 1.

19. An expression vector comprising the nucleic acid molecule of claim 18.

20. A host cell comprising the nucleic acid molecule of claim 18.
